(19) United States
(12) Patent Application Publication
(10) Pub. No.: US 2009/0291921 A1
(43) Pub. Date: Nov. 26, 2009

(54) INTEGRASE INHIBITORS

(75) Inventors: Salman Y. Jabri, Irvine, CA (US); Haolun Jin, Foster City, CA (US); Choung U. Kim, San Carlos, CA (US); Jiayao Li, Foster City, CA (US); Samuel E. Metobo, Newark, CA (US); Michael R. Mish, La Honda, CA (US)

Correspondence Address: GILEAD SCIENCES INC, 333 LAKESIDE DR, FOSTER CITY, CA, 94404, US

(73) Assignee: Gilead Sciences, Inc., Foster City, CA (US)

(21) Appl. No.: 12/274,107

(22) Filed: Nov. 19, 2008

Related U.S. Application Data
(60) Provisional application No. 60/989,406, filed on Nov. 20, 2007.

(51) Int. Cl. A61K 31/675; A61K 31/54; A61K 31/497; A61K 31/4745; C07F 9/28; C07D 471/00

(52) U.S. Cl. 514/81; 514/228.2; 514/253.03; 514/292; 544/58.2; 544/361; 546/23; 546/84

(57) ABSTRACT

Tricyclic compounds, protected intermediates thereof, and methods for inhibition of HIV-integrase are disclosed.

### CROSS-REFERENCE TO RELATED APPLICATIONS

This application claims priority to U.S. Provisional Application Ser. No. 60/989,406, filed Nov. 20, 2007. The contents of this provisional application is herein incorporated by reference in its entirety for all purposes.

### FIELD OF THE INVENTION

The invention relates generally to compounds having antiviral activity, and more specifically, compounds having HIV-integrase inhibitory properties.

### BACKGROUND OF THE INVENTION

Human immunodeficiency virus (HIV) infection and related diseases are a major public health problem worldwide. A virally encoded integrase protein mediates specific incorporation and integration of viral DNA into the host genome. Integration is necessary for viral replication. Accordingly, inhibition of HIV integrase is an important therapeutic pursuit for treatment of HIV infection of the related diseases.

Human immunodeficiency virus type 1 (HIV-1) encodes three enzymes which are required for viral replication: reverse transcriptase, protease, and integrase. Although drugs targeting reverse transcriptase and protease are in wide use and have shown effectiveness, particularly when employed in combination, toxicity and development of resistant strains have limited their usefulness (Palella, et al N. Engl. J. Med. (1998) 338:853-860; Richman, D. D. Nature (2001) 410:995-1001). There is a need for new agents directed against alternate sites in the viral life cycle. Integrase has emerged as an attractive target, because it is necessary for stable infection and homologous enzymes are lacking in the human host (LaFemina, et al J. Virol. (1992) 66:7414-7419). The function of integrase is to catalyze integration of proviral DNA, resulting from the reverse transcription of viral RNA, into the host genome, by a stepwise fashion of endonucleolytic processing of proviral DNA within a cytoplasmic preintegration complex (termed 3′-processing or “3′-P”) with specific DNA sequences at the end of the HIV-1 long terminal repeat (LTR) regions, followed by translocation of the complex into the nuclear compartment where integration of 3′-processed proviral DNA into host DNA occurs in a “strand transfer” (ST) reaction (Hazuda, et al Science (2000) 287:646-650; Katzman, et al Adv. Virus Res. (1999) 52:371-395; Asante-Applah, et al Ad. Virus Res. (1999) 52:351-369). Although numerous agents potently inhibit 3′-P and ST in extracellular assays that employ recombinant integrase and viral long-terminal-repeat oligonucleotide sequences, often such inhibitors lack inhibitory potency when assayed using fully assembled preintegration complexes or fail to show antiviral effects against HIV-infected cells (Pommier, et al Adv. Virus Res. (1999) 52:427-458; Farnet, et al Proc. Natl. Acad. Sci. U.S.A. (1996) 93:9742-9747; Pommier, et al Antiviral Res. (2000) 47:139-148.

HIV integrase inhibitory compounds with improved antiviral and pharmacokinetic properties are desirable, including enhanced activity against development of HIV resistance, improved oral bioavailability, greater potency and extended effective half-life in vivo (Nair, V. “HIV integrase as a target for antiviral chemotherapy” Reviews in Medical Virology (2002) 12(3):179-193). Three-dimensional quantitative structure-activity relationship studies and docking simulations (Buolamwini, et al Jour. Med. Chem. (2002) 45:841-852) of conformationally-restrained cinnamoyl-type integrase inhibitors (Artico, et al Jour. Med. Chem. (1998) 41:3948-3960) have correlated hydrogen-bonding interactions to the inhibitory activity differences among the compounds.

Certain HIV integrase inhibitors have been disclosed which seek to block integration in extracellular assays and exhibit antiviral effects against HIV-infected cells (Anthony, et al WO 02/30426; Anthony, et al WO 02/30930; Anthony, et al WO 02/30931; WO 02/055079; Zhuang, et al WO 02/36734; U.S. Pat. No. 6,395,743; U.S. Pat. No. 6,245,806; U.S. Pat. No. 6,271,402; Fujishita, et al WO 00/039086; Uenaka et al WO 00/075122; Selnick, et al WO 99/62513; Young, et al WO 99/62520; Payne, et al WO 01/00578; Jing, et al Biochemistry (2002) 41:5397-5403; Pais, et al J. Med. Chem. (2002) 45:3184-94; Goldgur, et al Proc. Natl. Acad. Sci. U.S.A. (1999) 96:13040-13043; Espeseth, et al Proc. Natl. Acad. Sci. U.S.A. (2000) 97:11244-11249). Recent HIV integrase inhibitors are shown in WO 2005/016927, WO 2004/096807, WO 2004/035577, WO 2004/035576 and US 2003/0055071.

There exists a need to find additional compounds for the treatment of HIV, particularly, improved integrase inhibitors having beneficial properties and good efficacy.

### SUMMARY OF THE INVENTION

One aspect the invention provides a compound of formula (I):

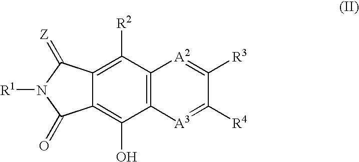

wherein:

A² is N or CRₐ;

A³ is N or CRₐ;

R¹ is H, R_{b}, or -Q-R_{c};

R² is C₁-C₆alkoxycarbonyl, —C(═O)C(═O)ORₐ, or —C(═O)NRₐR_{g};

or R² is C₁-C₆alkyl that is substituted with one or more groups independently selected from heterocycle, substituted heterocyle, —C(═O)ORₐ, —C(═N—ORₐ)—NRₑRₑ, —C(═NRₐ)—NRₑRₑ, —P(═O)(Rₙ)(Rₙ), —C(═O)N(R_{c})NRₑRₑ, —C(═O)NRₐR_{g}, and —C(═O)Rₕ;

or R² is C₃-C₈-carbocycle that is substituted with one or more groups independently selected from heterocycle, —C(═O)ORₐ, —C(═O)NRₑRₑ, —C(═O)N(Rₐ)—S(O)₂Rₐ, —C(═O)Rₕ,

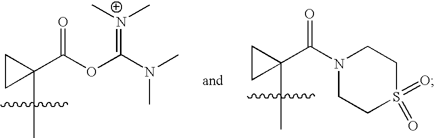

R³ is H, halo, or C₁-C₆alkyl that is optionally substituted with Rₖ;

R⁴ is H, halo, or C₁-C₆alkyl that is optionally substituted with Rₖ;

Q is C₁-C₆alkylene;

Z is O or two hydrogens;

each Rₐ is independently H or C₁-C₆alkyl;

R_{b} is C₁-C₆alkyl, C₂-C₆alkenyl, or C₂-C₆alkynyl, each of which is optionally substituted with one or more halo, hydroxy, C₁-C₆alkoxy, dimethylamino, diethylamino, N-ethyl-N-methylamino, morpholino, thiomorpholino, piperidino, or piperazino;

R_{c} is a C₃-C₁₂-carbocycle, a substituted C₃-C₁₂-carbocycle, aryl, substituted aryl, heteroaryl, or substituted heteroaryl;

each R_{d} is independently C₁-C₆alkyl;

each Rₑ is independently H, C₁-C₆alkyl, C₁-C₆alkoxy, C₂-C₆alkenyl, C₂-C₆alkynyl, C₃-C₁₂-carbocycle, wherein each C₁-C₆alkyl, C₁-C₆alkoxy, C₂-C₆alkenyl, C₃-C₁₂-carbocycle, and C₂-C₆alkynyl of Rₑ is optionally substituted with aryl, heteroaryl, substituted aryl, substituted heteroaryl, cyano, hydroxy, C₃-C₁₂-carbocycle, —C(O)ORₐ, —C(═O)C(O)ORₐ, —C(═O)NR_{g}R_{g}, —N(Rₐ)—C(═O)—Rₐ, —N(Rₐ)—S(O)₂—Rₐ heteroaryl, —S(O)₂—NR_{g}R_{g}, —C(═NRₘ)—NR_{g}R_{g}, or —C(═O)NR_{g}R_{g};

each R_{f} is independently H, C₁-C₆alkyl, phenyl, or phenylC₁-C₆alkyl, wherein any phenyl ring of R_{f} is optionally substituted with one or more fluoro, chloro, bromo, iodo, cyano, C₁-C₆alkyl, C₁-C₆alkyl-C(═O)—, C₁-C₆alkyl-S(O)₂—, —C(═O)NRₐRₐ, or —C(═O)ORₐ;

each R_{g} is independently —S(O)₂—Rₐ, heterocycle, substituted heterocycle, C₂-C₆alkynyl or each R_{g} is C₁-C₆alkyl or C₃-C₁₂-carbocycle, which C₁-C₆alkyl or C₃-C₁₂-carbocycle is substituted with one or more —C(═O)ORₐ, or —S(O)₂—NRₐRₐ;

each Rₕ is independently selected from:

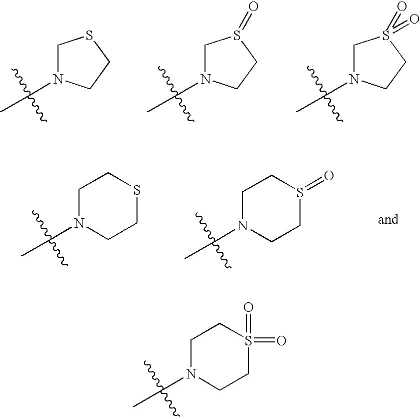

each Rₖ is phenyl, optionally substituted with one or more F, Cl, Br, I, hydroxy, cyano, trifluoromethyl, trifluoromethoxy, or C₁-C₆alkyl; and

each Rₘ is hydrogen, hydroxy, C₁-C₆alkyl, or C₁-C₆alkoxy;

each Rₙ is independently H, C₁-C₆alkyl, phenyl, phenylC₁-C₆alkyl, C₁-C₆alkoxy, phenoxy, or phenylC₁-C₆alkoxy, wherein any phenyl ring of Rₙ is optionally substituted with one or more groups independently selected from fluoro, chloro, bromo, iodo, cyano, C₁-C₆alkyl, C₁-C₆alkyl-C(O)—, C₁-C₆alkyl-S(O)₂—, —C(═O)NRₐRₐ, and —C(═O)ORₐ;

or a pharmaceutically acceptable salt or prodrug thereof.

The invention also includes a pharmaceutical composition comprising a therapeutically effective amount of a compound of the invention, or a pharmaceutically acceptable salt thereof, in combination with a pharmaceutically acceptable diluent, excipient or carrier.

The invention also includes a pharmaceutical composition comprising a therapeutically effective amount of a compound of the invention, or a pharmaceutically acceptable salt thereof, in combination with a booster agent and/or a therapeutically effective amount of one or more of the following agents: another compound of the invention, an AIDS treatment agent, such as an HIV inhibitor agent, an anti-infective agent or an immunomodulator agent. The HIV inhibitor agent may include an HIV-protease inhibitor, a nucleoside reverse transcriptase inhibitor, a non-nucleoside reverse transcriptase inhibitor or a mixture thereof.

The invention also includes methods of treating (for example, preventing, mediating, inhibiting, etc.) the proliferation of HIV virus, treating AIDS, delaying the onset of AIDS or ARC symptoms and generally inhibiting HIV integrase. The methods comprise administering to a mammal in need of such treatment an effective amount of a compound of the invention (e.g. an amount effective to inhibit the growth of HIV infected cells of the mammal).

In another aspect of the invention, the activity of HIV integrase is inhibited by a method comprising the step of treating a mammal or sample suspected of containing HIV virus with a compound or composition of the invention.

The invention also includes processes and novel intermediates disclosed herein which are useful for preparing compounds of the invention. Some of the compounds of the invention are useful to prepare other compounds of the invention.

This invention also includes a method of increasing cellular accumulation, bioavailability or retention of drug compounds, thus improving their therapeutic and diagnostic value, by administering a phosphonate prodrug form of a compound of the invention.

In other aspects, methods for the synthesis, analysis, separation, isolation, crystallization, purification, characterization, resolution of isomers (including enantiomers and diastereomers) and testing of the compounds of the invention are provided.

The invention, in part, provides compounds possessing improved anti-HIV and/or pharmaceutical properties.

### DEFINITIONS

Unless stated otherwise, the following terms and phrases as used herein are intended to have the following meanings:

The terms “phosphonate” and “phosphonate group” mean a functional group or moiety within a molecule that comprises at least one phosphorus-carbon bond, and at least one phosphorus-oxygen double bond. The phosphorus atom is further substituted with oxygen, sulfur, and nitrogen substituents. These substituents may be part of a prodrug moiety. As defined herein, “phosphonate” and “phosphonate group” include molecules with phosphonic acid, phosphonic monoester, phosphonic diester, phosphonamidate, phosphondiamidate, and phosphonthioate functional groups.

The term “prodrug” as used herein refers to any compound that when administered to a biological system generates the drug substance, i.e. active ingredient, as a result of spontaneous chemical reaction(s), enzyme catalyzed chemical reaction(s), photolysis, and/or metabolic chemical reaction(s). A prodrug is thus a covalently modified analog or latent form of a therapeutically-active compound.

“Pharmaceutically acceptable prodrug” refers to a compound that is metabolized in the host, for example hydrolyzed or oxidized, by either enzymatic action or by general acid or base solvolysis, to form an active ingredient. Typical examples of prodrugs of the compounds of the invention have biologically labile protecting groups on a functional moiety of the compound. Prodrugs include compounds that can be oxidized, reduced, aminated, deaminated, esterified, deesterified, alkylated, dealkylated, acylated, deacylated, phosphorylated, dephosphorylated, photolyzed, hydrolyzed, or other functional group change or conversion involving forming or breaking chemical bonds on the prodrug.

“Prodrug moiety” means a labile functional group which separates from the active inhibitory compound during metabolism, systemically, inside a cell, by hydrolysis, enzymatic cleavage, or by some other process (Bundgaard, H., “Design and Application of Prodrugs” in Textbook of Drug Design and Development (1991), P. Krogsgaard-Larsen and H. Bundgaard, Eds. Harwood Academic Publishers, pp. 113-191). Enzymes which are capable of an enzymatic activation mechanism with the prodrug compounds of the invention include, but are not limited to, amidases, esterases, microbial enzymes, phospholipases, cholinesterases, and phosphases. Prodrug moieties can serve to enhance solubility, absorption and lipophilicity to optimize drug delivery, bioavailability and efficacy. A “prodrug” is thus a covalently modified analog of a therapeutically-active compound.

Exemplary prodrug moieties include the hydrolytically sensitive or labile acyloxymethyl esters —CH₂C(═O)R²⁰ and acyloxymethyl carbonates —CH₂OC(═O)OR²⁰ where R²⁰ is C₁-C₆ alkyl, C₁-C₆ substituted alkyl, C₆-C₂₀ aryl or C₆-C₂₀ substituted aryl. The acyloxyalkyl ester was first used as a prodrug strategy for carboxylic acids and then applied to phosphates and phosphonates by Farquhar et al., (1983) J. Pharm. Sci. 72: 324; also U.S. Pat. Nos. 4,816,570, 4,968,788, 5,663,159 and 5,792,756, which are all incorporated by reference. In certain compounds of the invention, a prodrug moiety is part of a phosphonate group. Subsequently, the acyloxyalkyl ester was used to deliver phosphonic acids across cell membranes and to enhance oral bioavailability. A close variant of the acyloxyalkyl ester, the alkoxycarbonyloxyalkyl ester (carbonate), may also enhance oral bioavailability as a prodrug moiety in the compounds of the invention. An exemplary acyloxymethyl ester is pivaloyloxymethoxy, (POM)-CH₂C(═O)C(CH₃)₃. An exemplary acyloxymethyl carbonate prodrug moiety is pivaloyloxymethylcarbonate (POC) —CH₂OC(═O)OC(CH₃)₃.

The phosphonate group may be a phosphonate prodrug moiety. The prodrug moiety may be sensitive to hydrolysis, such as, but not limited to a pivaloyloxymethyl carbonate (POC) or POM group. Alternatively, the prodrug moiety may be sensitive to enzymatic potentiated cleavage, such as a lactate ester or a phosphonamidate-ester group. Exemplary phosphonate prodrug moieties include by way of example and not limitation groups of the structure A⁵ as described herein.

Aryl esters of phosphorus groups, especially phenyl esters, are reported to enhance oral bioavailability (DeLambert et al (1994) J. Med. Chem. 37: 498). Phenyl esters containing a carboxylic ester ortho to the phosphate have also been described (Khamnei and Torrence, (1996) J. Med. Chem. 39:4109-4115). Benzyl esters are reported to generate the parent phosphonic acid. In some cases, substituents at the ortho- or para-position may accelerate the hydrolysis. Benzyl analogs with an acylated phenol or an alkylated phenol may generate the phenolic compound through the action of enzymes, e.g. esterases, oxidases, etc., which in turn undergoes cleavage at the benzylic C—O bond to generate the phosphoric acid and the quinone methide intermediate. Examples of this class of prodrugs are described by Mitchell et al., (1992) J. Chem. Soc. Perkin Trans. I 2345; Brook et al., WO 91/19721. Still other benzylic prodrugs have been described containing a carboxylic ester-containing group attached to the benzylic methylene (Glazier et al., WO 91/19721). Thio-containing prodrugs are reported to be useful for the intracellular delivery of phosphonate drugs. These proesters contain an ethylthio group in which the thiol group is either esterified with an acyl group or combined with another thiol group to form a disulfide. Deesterification or reduction of the disulfide generates the free thio intermediate which subsequently breaks down to the phosphoric acid and episulfide (Puech et al., (1993) Antiviral Res., 22: 155-174; Benzaria et al., (1996) J. Med. Chem. 39: 4958). Cyclic phosphonate esters have also been described as prodrugs of phosphorus-containing compounds (Erion et al., U.S. Pat. No. 6,312,662).

“Protecting group” refers to a moiety of a compound that masks or alters the properties of a functional group or the properties of the compound as a whole. The chemical substructure of a protecting group varies widely. One function of a protecting group is to serve as intermediates in the synthesis of the parental drug substance. Chemical protecting groups and strategies for protection/deprotection are well known in the art. See. “Protective Groups in Organic Chemistry”, Theodora W. Greene (John Wiley & Sons, Inc., New York, 1991, which is incorporated herein by reference. Protecting groups are often utilized to mask the reactivity of certain functional groups, to assist in the efficiency of desired chemical reactions, e.g. making and breaking chemical bonds in an ordered and planned fashion. Protection of functional groups of a compound alters other physical properties besides the reactivity of the protected functional group, such as the polarity, lipophilicity (hydrophobicity), and other properties which can be measured by common analytical tools. Chemically protected intermediates may themselves be biologically active or inactive.

The term “hydroxyl protecting group,” as used herein, refers to an easily removable group which is known in the art to protect a hydroxyl group against undesirable reaction during synthetic procedures and/or during biodelivery and which group can be selectively removed. The use of hydroxy-protecting groups is well known in the art for protecting groups and many such protecting groups are known, for example, T. H. Greene and P. G. M. Wuts, Protective Groups in Organic Synthesis, 2nd edition, John Wiley & Sons, New York (1991). Examples of hydroxy-protecting groups include, but are not limited to,

Ethers (methyl);

Substituted methyl ethers (methoxymethyl, methylthiomethyl, t-butylthiomethyl, (phenyldimethylsilyl)methoxymethyl, benzyloxymethyl, p-methoxybenzyloxymethyl, (4-methoxyphenoxy)methyl, guaiacolmethyl, t-butoxymethyl, 4-pentenyloxymethyl, siloxymethyl, 2-methoxyethoxymethyl, 2,2,2-trichloroethoxymethyl, bis(2-chloroethoxy)methyl, 2-(trimethylsilyl)ethoxymethyl, tetrahydropyranyl, 3-bromotetrahydropyranyl, tetrahydrothiopyranyl, 1-methoxycyclohexyl, 4-methoxytetrahydropyranyl, 4-methoxytetrahydro-thiopyranyl, 4-methoxytetrahydropthiopyranyl S,S-dioxido, 1->(2-chloro-4-methyl)phenyl-4-methoxypiperidin-4-yl, 1,4-dioxan-2-yl, tetrahydrofuranyl, tetrahydrothiofuranyl, 2,3,3a,4,5,6,7,7a-octahydro-7,8,8-trim ethyl-4,7-methanobenzofuran-2-yl));

Substituted ethyl ethers (1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl);

Substituted benzyl ethers (p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2- and 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxy)phenyldiphenylmethyl, 4,4′,4″-tris(4,5-dichlorophthalimidophenyl)methyl, 4,4′,41″-tris(levulinoyloxyphenyl)-methyl, 4,4′,4″-tris(benxoyloxyphenyl)methyl, 3-(imidazol-1-ylmethyl)bis(4′,4″-dimethoxyphenyl)methyl, 1,1-bis(4-methoxyphenyl)-1-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-Dioxido);

Silyl ethers (trimethylsilyl, triethylsilyl, triisopropylsilyl, dimethylisopropylsilyl, diethylisopropylsilyl, dimethylthexylsilyl, t-butyldimethyl-silyl, t-butyldiphenylsilyl, tribenzylsilyl, tri-p-xylylsilyl, triphenylsilyl, diphenylmethylsilyl, t-butylmethoxyphenylsilyl);

Esters (formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, p-poly-phenylacetate, 3-phenyl-propionate, 4-oxopentanoate (Levulinate), 4,4-(ethylenedithio)pentanoate, pivaloate, adamantoate, crotonate, 4-methoxycrotonate, benzoate, p-phenyl-benzoate, 2,4,6-trimethylbenzoate (Mesitoate));

Carbonates (methyl, 9-fluorenylmethyl, ethyl, 2,2,2-trichloroethyl, 2-(trimethylsilyl)ethyl, 2-(phenylsulfonyl)ethyl, 2-(triphenylphosphonio)ethyl, isobutyl, vinyl, allyl, p-nitrophenyl, benzyl, p-methoxybenzyl, 3,4-dimethoxybenzyl, o-nitrobenzyl, p-nitrobenzyl, S-benzyl thiocarbonate, 4-ethoxy-1-naphthyl, methyl dithiocarbonate);

Groups with assisted cleavage (2-iodobenzoate, 4-azidobutyrate, 4-nitro-4-methylpentanoate, o-(dibromomethyl)benzoate, 2-formylbenzenesulfonate, 2-(methylthiomethoxy)ethyl carbonate, 4-(methylthiomethoxy)butyrate, 2-(methylthiomethoxymethyl)benzoate);

Miscellaneous Esters (2,6-dichloro-4-methylphenoxyacetate, 2,6-dichloro-4-(1,1,3,3-tetramethylbutyl)phenoxyacetate, 2,4-bis(1,1-dimethylpropyl)-phenoxyacetate, chlorodiphenylacetate, isobutyrate, monosuccinoate, (E)-2-methyl-2-butenoate (Tigloate), o-(methoxycarbonyl)benzoate, p-poly-benzoate, a-naphthoate, nitrate, alkyl N,N,N′,N′-tetramethylphosphorodiamidate, N-phenylcarbamate, borate, dimethylphosphinothioyl, 2,4-dinitrophenylsulfenate); and

Sulfonates (sulfate, methanesulfonate (Mesylate), benzylsulfonate, Tosylate).

More typically, hydroxy protecting groups include substituted methyl ethers, substituted benzyl ethers, silyl ethers, and esters including sulfonic acid esters, still more typically, trialkylsilyl ethers, tosylates and acetates.

The term “amino protecting group,” as used herein, refers to an easily removable group which is known in the art to protect an amino group against undesired reaction during synthetic procedures and/or during biodelivery and which group can be selectively removed. Such protecting groups are described by Greene at pages 315-385. They include:

Carbamates (methyl and ethyl, 9-fluorenylmethyl, 9(2-sulfo)fluoroenyl-methyl, 9-(2,7-dibromo)fluorenylmethyl, 2,7-di-t-buthyl-[9-(10,10-dioxo-10,10,10,10-tetrahydrothioxanthyl)]methyl, 4-methoxyphenacyl);

Substituted ethyl (2,2,2-trichoroethyl, 2-trimethylsilylethyl, 2-phenylethyl, 1-(1-adamantyl)-1-methylethyl, 1,1-dimethyl-2-haloethyl, 1,1-dimethyl-2,2-dibromoethyl, 1,1-dimethyl-2,2,2-trichloroethyl, 1-methyl-1-(4-biphenylyl)ethyl, 1-(3,5-di-t-butylphenyl)-1-methylethyl, 2-(2′- and 4′-pyridylethyl, 2-(N,N-dicyclohexylcarboxamido)ethyl, t-butyl, 1-adamantyl, vinyl, allyl, 1-isopropylallyl, cinnamyl, 4-nitrocinnamyl, S-quinolyl, N-hydroxypiperidinyl, alkyldithio, benzyl, p-methoxybenzyl, p-nitrobenzyl, p-bromobenzyl, p-chlorobenzyl, 2,4-dichlorobenzyl, 4-methylsulfinylbenzyl, 9-anthrylmethyl, diphenylmethyl);

Groups With Assisted Cleavage (2-methylthioethyl, 2-methylsulfonylethyl, 2-(p-toluenesulfonyl)ethyl, [2-(1,3-dithianyl)]methyl, 4-methylthiophenyl, 2,4-dimethylthiophenyl, 2-phosphonioethyl, 2-triphenylphosphonioisopropyl, 1,1-dimethyl-2-cyanoethyl, m-choro-p-acyloxybenzyl, p-(dihydroxyboryl)benzyl, 5-benzisoxazolylmethyl, 2-(trifluoromethyl)-6-chromonylmethyl);

Groups Capable of Photolytic Cleavage (m-nitrophenyl, 3,5-dimethoxybenzyl, o-nitrobenzyl, 3,4-dimethoxy-6-nitrobenzyl, phenyl(o-nitrophenyl)methyl);

Urea-Type Derivatives (phenothiazinyl-(10)-carbonyl, N′-p-toluenesulfonylaminocarbonyl, N′-phenylaminothiocarbonyl);

Miscellaneous Carbamates (t-amyl, S-benzyl thiocarbamate, p-cyanobenzyl, cyclobutyl, cyclohexyl, cyclopentyl, cyclopropylmethyl, p-decyloxybenzyl, diisopropylmethyl, 2,2-dimethoxycarbonylvinyl, o-(N,N-dimethyl-carboxamido)benzyl, 1,1-dimethyl-3-(N,N-dimethylcarboxamido)propyl, 1,1-dimethylpropynyl, di(2-pyridyl)methyl, 2-furanylmethyl, 2-Iodoethyl, Isobornyl, Isobutyl, Isonicotinyl, p-(p′-Methoxyphenylazo)benzyl, 1-methylcyclobutyl, 1-methylcyclohexyl, 1-methyl-1-cyclopropylmethyl, 1-methyl-1-(3,5-dimethoxyphenyl)ethyl, 1-methyl-1-(p-phenylazophenyl)ethyl, 1-methyl-1-phenylethyl, 1-methyl-1-(4-pyridyl)ethyl, phenyl, p-(phenylazo)benzyl, 2,4,6-tri-t-butylphenyl, 4-(trimethylammonium)benzyl, 2,4,6-trimethylbenzyl);

Amides (N-formyl, N-acetyl, N-choroacetyl, N-trichoroacetyl, N-trifluoroacetyl, N-phenylacetyl, N-3-phenylpropionyl, N-picolinoyl, N-3-pyridylcarboxamide, N-benzoylphenylalanyl, N-benzoyl, N-p-phenylbenzoyl); Amides With Assisted Cleavage (N-o-nitrophenylacetyl, N-o-nitrophenoxyacetyl, N-acetoacetyl, (N′-dithiobenzyloxycarbonylamino)acetyl, N-3-(p-hydroxyphenyl)propionyl, N-3-(o-nitrophenyl)propionyl, N-2-methyl-2-(o-nitrophenoxy)propionyl, N-2-methyl-2-(o-phenylazophenoxy)propionyl, N-4-chlorobutyryl, N-3-methyl-3-nitrobutyryl, N-o-nitrocinnamoyl, N-acetylmethionine, N-o-nitrobenzoyl, N-o-(benzoyloxymethyl)benzoyl, 4,5-diphenyl-3-oxazolin-2-one);

Cyclic Imide Derivatives (N-phthalimide, N-dithiasuccinoyl, N-2,3-diphenylmaleoyl, N-2,5-dimethylpyrrolyl, N-1,1,4,4-tetramethyldisilylazacyclopentane adduct, 5-substituted 1,3-dimethyl-1,3,5-triazacyclohexan-2-one, 5-substituted 1,3-dibenzyl-1,3-5-triazacyclohexan-2-one, 1-substituted 3,5-dinitro-4-pyridonyl);

N-Alkyl and N-Aryl Amines (N-methyl, N-allyl, N-[2-(trimethylsilyl)ethoxy]methyl, N-3-acetoxypropyl, N-(1-isopropyl-4-nitro-2-oxo-3-pyrrolin-3-yl),

Quaternary Ammonium Salts, N-benzyl, N-di(4-methoxyphenyl)methyl, N-5-dibenzosuberyl, N-triphenylmethyl, N-(4-methoxyphenyl)diphenylmethyl, N-9-phenylfluorenyl, N-2,7-dichloro-9-fluorenylmethylene, N-ferrocenylmethyl, N-2-picolylamine N′-oxide),

Imine Derivatives (N-1,1-dimethylthiomethylene, N-benzylidene, N-p-methoxybenzylidene, N-diphenylmethylene, N-[(2-pyridyl)mesityl]methylene, N,(N′,N′-dimethylaminomethylene, N,N′-isopropylidene, N-p-nitrobenzylidene, N-salicylidene, N-5-chlorosalicylidene, N-(5-chloro-2-hydroxyphenyl)phenyl-methylene, N-cyclohexylidene); Enamine Derivatives (N-(5,5-dimethyl-3-oxo-1-cyclohexenyl));

N-Metal Derivatives (N-borane derivatives, N-diphenylborinic acid derivatives, N-[phenyl(pentacarbonylchromium- or -tungsten)]carbenzyl, N-copper or N-zinc chelate);

N—N Derivatives (N-nitro, N-nitroso, N-oxide); N—P Derivatives (N-diphenylphosphinyl, N-dimethylthiophosphinyl, N-diphenylthiophosphinyl, N-dialkyl phosphoryl, N-dibenzyl phosphoryl, N-diphenyl phosphoryl);

N—Si Derivatives; N—S Derivatives; N-Sulfenyl Derivatives (N-benzenesulfenyl, N-o-nitrobenzenesulfenyl, N-2,4-dinitrobenzenesulfenyl, N-pentachlorobenzenesulfenyl, N-2-nitro-4-methoxybenzenesulfenyl, N-triphenylmethylsulfenyl, N-3-nitropyridinesulfenyl); and

N-sulfonyl Derivatives (N-p-toluenesulfonyl, N-benzenesulfonyl, N-2,3,6-trimethyl-4-methoxybenzenesulfonyl, N-2,4,6-trimethoxybenzenesulfonyl, N-2,6-dimethyl-4-methoxybenzenesulfonyl, N-pentamethylbenzenesulfonyl, N-2,3,5,6-tetramethyl-4-methoxybenzenesulfonyl, N-4-methoxybenzenesulfonyl, N-2,4,6-trimethylbenzenesulfonyl, N-2,6-dimethoxy-4-methylbenzenesulfonyl, N-2,2,5,7,8-pentamethylchroman-6-sulfonyl, N-methanesulfonyl, N-.beta.-trimethylsilyl-ethanesulfonyl, N-9-anthracenesulfonyl, N-4-(4′,8′-dimethoxynaphthyl-methyl)benzenesulfonyl, N-benzylsulfonyl, N-trifluoromethylsulfonyl, N-phenacylsulfonyl).

Protected compounds may also exhibit altered, and in some cases, optimized properties in vitro and in vivo, such as passage through cellular membranes and resistance to enzymatic degradation or sequestration. In this role, protected compounds with intended therapeutic effects may be referred to as prodrugs. Another function of a protecting group is to convert the parental drug into a prodrug, whereby the parental drug is released upon conversion of the prodrug in vivo. Because active prodrugs may be absorbed more effectively than the parental drug, prodrugs may possess greater potency in vivo than the parental drug. Protecting groups are removed either in vitro, in the instance of chemical intermediates, or in vivo, in the case of prodrugs. With chemical intermediates, it is not particularly important that the resulting products after deprotection, e.g. alcohols, be physiologically acceptable, although in general it is more desirable if the products are pharmacologically innocuous. Exemplary protecting groups include by way of example and not limitation groups of the structure R^{X} other than hydrogen.

Examples of physiologically acceptable salts of the compounds of the invention include salts derived from an appropriate base, such as an alkali metal (for example, sodium), an alkaline earth (for example, magnesium), ammonium and NX₄⁺ (wherein X is C₁-C₄ alkyl). Physiologically acceptable salts of a hydrogen atom or an amino group include salts of organic carboxylic acids such as acetic, benzoic, lactic, fumaric, tartaric, maleic, malonic, malic, isethionic, lactobionic and succinic acids; organic sulfonic acids, such as methanesulfonic, ethanesulfonic, benzenesulfonic and p-toluenesulfonic acids; and inorganic acids, such as hydrochloric, sulfuric, phosphoric and sulfamic acids. Physiologically acceptable salts of a compound having a hydroxy group include the anion of said compound in combination with a suitable cation such as Na⁺ and NX₄⁺ (wherein X is independently selected from the group consisting of H and a C₁-C₄ alkyl group).

For therapeutic use, salts of active ingredients of the compounds of the invention will be physiologically acceptable, i.e. they will be salts derived from a physiologically acceptable acid or base. However, salts of acids or bases which are not physiologically acceptable may also find use, for example, in the preparation or purification of a physiologically acceptable compound. All salts, whether or not derived form a physiologically acceptable acid or base, are within the scope of the present invention.

“Alkyl” is C₁-C₁₈ hydrocarbon containing normal, secondary, tertiary or cyclic carbon atoms. Examples are methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (1-Bu, 1-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃.

“Alkenyl” is C₂-C₁₈ hydrocarbon containing normal, secondary, tertiary or cyclic carbon atoms with at least one site of unsaturation, i.e. a carbon-carbon, sp² double bond. Examples include, but are not limited to: ethylene or vinyl (—CH═CH₂), allyl (—CH₂CH═CH₂), cyclopentenyl (—C₅H₇), and 5-hexenyl (—CH₂CH₂CH₂CH₂CH═CH₂).

“Alkynyl” is C₂-C₁₈ hydrocarbon containing normal, secondary, tertiary or cyclic carbon atoms with at least one site of unsaturation, i.e. a carbon-carbon, sp triple bond. Examples include, but are not limited to: acetylenic (—C≡CH) and propargyl (—CH₂C≡CH),

The terms “alkylene” and “alkyldiyl” each refer to a saturated, branched or straight chain or cyclic hydrocarbon radical of 1-18 carbon atoms, and having two monovalent radical centers derived by the removal of two hydrogen atoms from the same or two different carbon atoms of a parent alkane. Typical alkylene radicals include, but are not limited to: methylene (—CH₂—), methylmethylene (—C(CH₃)H—) 1,2-ethyl (—CH₂CH₂—), 1,3-propyl (—CH₂CH₂CH₂—), 1,4-butyl (—CH₂CH₂CH₂CH₂—), and the like.

“Alkenylene” refers to an unsaturated, branched or straight chain or cyclic hydrocarbon radical of 2-18 carbon atoms, and having two monovalent radical centers derived by the removal of two hydrogen atoms from the same or two different carbon atoms of a parent alkene, i.e. double carbon-carbon bond moiety. Typical alkenylene radicals include, but are not limited to: 1,2-ethylene (—CH═CH—).

“Alkynylene” refers to an unsaturated, branched or straight chain or cyclic hydrocarbon radical of 2-18 carbon atoms, and having two monovalent radical centers derived by the removal of two hydrogen atoms from the same or two different carbon atoms of a parent alkyne, i.e. triple carbon-carbon bond moiety. Typical alkynylene radicals include, but are not limited to: acetylene (—C≡C—), propargyl (—CH₂C≡C—), and 4-pentynyl (—CH₂CH₂CH₂C≡CH—).

“Aryl” means a monovalent aromatic hydrocarbon radical of 6-20 carbon atoms derived by the removal of one hydrogen atom from a single carbon atom of a parent aromatic ring system. Typical aryl groups include, but are not limited to, radicals derived from benzene, substituted benzene, naphthalene, anthracene, biphenyl, and the like.

“Heteroaryl” means a monovalent aromatic radical of one or more carbon atoms and one or more atoms selected from the group consisting of N, O, S and P, derived by the removal of one hydrogen atom from a single atom of a parent aromatic ring system. Heteroaryl groups may be a monocycle having 3 to 7 ring members (2 to 6 carbon atoms and 1 to 3 heteroatoms selected from the group consisting of N, O, P and S) or a bicycle having 7 to 10 ring members (4 to 9 carbon atoms and 1 to 3 heteroatoms selected from the group consisting of N, O, P and S). Heteroaryl bicycles have 7 to 10 ring atoms (6 to 9 carbon atoms and 1 to 2 heteroatoms selected from the group consisting of N, O and S) arranged as a bicyclo [4,5], [5,5], [5,6], or [6,6] system; or 9 to 10 ring atoms (8 to 9 carbon atoms and 1 to 2 hetero atoms selected from the group consisting of N and S) arranged as a bicyclo [5,6] or [6,6] system. The heteroaryl group may be bonded to the drug scaffold through a carbon, nitrogen, sulfur, phosphorus or other atom by a stable covalent bond.

Heteroaryl groups include, for example: pyridyl, dihydropyridyl isomers, pyridazinyl, pyrimidinyl, pyrazinyl, s-triazinyl, oxazolyl, imidazolyl, thiazolyl, isoxazolyl, pyrazolyl, isothiazolyl, furanyl, thiofuranyl, thienyl, and pyrrolyl.

“Arylalkyl” refers to an acyclic alkyl radical in which one of the hydrogen atoms bonded to a carbon atom, typically a terminal or sp³ carbon atom, is replaced with an aryl radical. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. The arylalkyl group comprises 6 to 20 carbon atoms, e.g. the alkyl moiety, including alkanyl, alkenyl or alkynyl groups, of the arylalkyl group is 1 to 6 carbon atoms and the aryl moiety is 5 to 14 carbon atoms.

Substituted substituents such as “substituted alkyl”, “substituted aryl”, “substituted heteroaryl”, “substituted heterocyclic” and “substituted arylalkyl” mean alkyl, aryl, heteroaryl, heterocyclic and arylalkyl respectively, in which one or more hydrogen atoms are each independently replaced with a substituent. Typical substituents include, but are not limited to, —X, —R, ═O, —O⁻, —OR, —S⁻, —SR, —NR₂, —NR₃, ═NR, —CX₃, —CN, —OCN, —SCN, —N═C═O, —NCS, —NO, —NO₂, ═N₂, —N₃, NC(═O)R, —C(═O)R, —C(═O)NRR—S(═O)₂O⁻, —S(═O)₂OH, —S(═O)₂R, —OS(═O)₂OR, —S(═O)₂NR, —S(═O)R, —OP(═O)O₂RR, —P(═O)O₂RR—P(═O)(O⁻)₂, —P(═O)(OH)₂, —C(═O)R, —C(═O)X, —C(S)R, —C(O)OR, —C(O)O, —C(S)OR, —C(O)SR, —C(S)SR, —C(O)NRR, —C(S)NRR, —C(NR)NRR, where each X is independently a halogen: F, Cl, Br, or I; and each R is independently H, alkyl, aryl, heterocycle, protecting group or prodrug moiety. Alkylene, alkenylene, and alkynylene groups may also be similarly substituted.

“Heterocycle” means a saturated, unsaturated or aromatic ring system including at least one N, O, S, or P. Heterocycle thus include heteroaryl groups. Heterocycle as used herein includes by way of example and not limitation these heterocycles described in Paquette, Leo A. “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; Katritzky, Alan R., Rees, C. W. and Scriven, E. “Comprehensive Heterocyclic Chemistry” (Pergamon Press, 1996); and J. Am. Chem. Soc. (1960) 82:5566.

Examples of heterocycles include by way of example and not limitation pyridyl, dihydropyridyl, tetrahydropyridyl (piperidyl), thiazolyl, tetrahydrothiophenyl, sulfur oxidized tetrahydrothiophenyl, pyrimidinyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, piperidinyl, 4-piperidonyl, pyrrolidinyl, 2-pyrrolidonyl, pyrrolinyl, tetrahydrofuranyl, bis-tetrahydrofuranyl, tetrahydropyranyl, bis-tetrahydropyranyl, tetrahydroquinolinyl, tetrahydroisoquinolinyl, decahydroquinolinyl, octahydroisoquinolinyl, azocinyl, triazinyl, 6H-1,2,5-thiadiazinyl, 2H,6H-1,5,2-dithiazinyl, thienyl, thianthrenyl, pyranyl, isobenzofuranyl, chromenyl, xanthenyl, phenoxathinyl, 2H-pyrrolyl, isothiazolyl, isoxazolyl, pyrazinyl, pyridazinyl, indolizinyl, isoindolyl, 3H-indolyl, 1H-indazoly, purinyl, 4H-quinolizinyl, phthalazinyl, naphthyridinyl, quinoxalinyl, quinazolinyl, cinnolinyl, pteridinyl, 4H-carbazolyl, carbazolyl, 13-carbolinyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, piperazinyl, indolinyl, isoindolinyl, quinuclidinyl, morpholinyl, oxazolidinyl, benzotriazolyl, benzisoxazolyl, oxindolyl, benzoxazolinyl, and isatinoyl.

One embodiment of the bis-tetrahydrofuranyl group is:

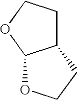

By way of example and not limitation, carbon bonded heterocycles are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline. Still more typically, carbon bonded heterocycles include 2-pyridyl, 3-pyridyl, 4-pyridyl, 5-pyridyl, 6-pyridyl, 3-pyridazinyl, 4-pyridazinyl, 5-pyridazinyl, 6-pyridazinyl, 2-pyrimidinyl, 4-pyrimidinyl, 5-pyrimidinyl, 6-pyrimidinyl, 2-pyrazinyl, 3-pyrazinyl, 5-pyrazinyl, 6-pyrazinyl, 2-thiazolyl, 4-thiazolyl, or 5-thiazolyl.

By way of example and not limitation, nitrogen bonded heterocycles are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or β-carboline. Still more typically, nitrogen bonded heterocycles include 1-aziridyl, 1-azetedyl, 1-pyrrolyl, 1-imidazolyl, 1-pyrazolyl, and 1-piperidinyl.

“Carbocycle” means a saturated or partially unsaturated ring system having 3 to 7 carbon atoms as a monocycle or 7 to 12 carbon atoms as a bicycle. Monocyclic carbocycles have 3 to 6 ring atoms, still more typically 5 or 6 ring atoms. Bicyclic carbocycles have 7 to 12 ring atoms, e.g. arranged as a bicyclo [4,5], [5,5], [5,6] or [6,6] system, or 9 or 10 ring atoms arranged as a bicyclo [5,6] or [6,6] system. Examples of monocyclic carbocycles include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, and spiryl.

The term “chiral” refers to molecules which have the property of non-superimposability of the mirror image partner, while the term “achiral” refers to molecules which are superimposable on their mirror image partner.

The term “stereoisomers” refers to compounds which have identical chemical constitution, but differ with regard to the arrangement of the atoms or groups in space.

“Diastereomer” refers to a stereoisomer with two or more centers of chirality and whose molecules are not mirror images of one another. Diastereomers have different physical properties, e.g. melting points, boiling points, spectral properties, and reactivities. Mixtures of diastereomers may separate under high resolution analytical procedures such as electrophoresis and chromatography.

“Enantiomers” refer to two stereoisomers of a compound which are non-superimposable mirror images of one another.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., Stereochemistry of Organic Compounds (1994) John Wiley & Sons, Inc., New York. Many organic compounds exist in optically active forms, i.e., they have the ability to rotate the plane of plane-polarized light. In describing an optically active compound, the prefixes D and L or R and S are used to denote the absolute configuration of the molecule about its chiral center(s). The prefixes d and l or (+) and (−) are employed to designate the sign of rotation of plane-polarized light by the compound, with (−) or l meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity.

### DETAILED DESCRIPTION OF EXEMPLARY EMBODIMENTS

The invention also provides compounds of formula I that are attached to one or more phosphonate groups or phosphonate prodrug groups. Such compounds can be prepared by removing one or more hydrogen atoms from a compound of formula I and by replacing that hydrogen atom with a group A⁵, wherein each A⁵ is independently:

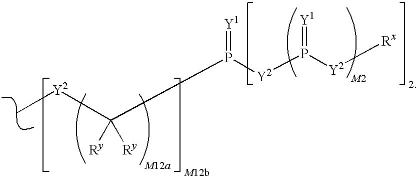

Y¹ is independently O, S, N(R^{x}), N(O)(R^{x}), N(OR^{x}), N(O)(OR^{x}), or N(N(R^{x})₂.

Y² is independently a bond, O, N(R^{x}), N(O)(R^{x}), N(OR^{x}), N(O)(OR^{x}), N(N(R^{x})₂), —S(═O)— (sulfoxide), —S(═O)₂— (sulfone), —S-(sulfide), or —S—S-(disulfide).

M2 is 0, 1 or 2.

M12a is 1, 2, 3, 4, 5, 6, 7, 8, 9, 10, 11, or 12.

M12b is 0, 1, 2, 3, 4, 5, 6, 7, 8, 9, 10, 11, or 12.

R^{y} is independently H, C₁-C₆ alkyl, C₁-C₆ substituted alkyl, aryl, substituted aryl, or a protecting group. Alternatively, taken together at a carbon atom, two vicinal R^{y} groups form a ring, i.e. a spiro carbon. The ring may be all carbon atoms, for example, cyclopropyl, cyclobutyl, cyclopentyl, or cyclohexyl, or alternatively, the ring may contain one or more heteroatoms, for example, piperazinyl, piperidinyl, pyranyl, or tetrahydrofuryl.

R^{x} is independently H, C₁-C₆ alkyl, C₁-C₆ substituted alkyl, C₆-C₂₀ aryl, C₆-C₂₀ substituted aryl, or a protecting group, or the formula:

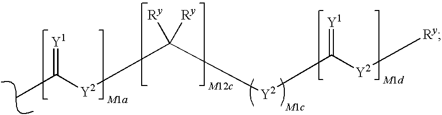

M1a, M1c, and M1d are independently 0 or 1.

M12c is 0, 1, 2, 3, 4, 5, 6, 7, 8, 9, 10, 11 or 12.

A linker may be interposed between the compound of formula I, II, or III, and each substituent A⁵. The linker may be O, S, NR, N—OR, C₁-C₁₂ alkylene, C₁-C₁₂ substituted alkylene, C₂-C₁₂ alkenylene, C₂-C₁₂ substituted alkenylene, C₂-C₁₂ alkynylene, C₂-C₁₂ substituted alkynylene, C(═O)NH, C(═O), S(═O)₂, C(═O)NH(CH₂)ₙ, and (CH₂CH₂O)ₙ, where n may be 1, 2, 3, 4, 5, or 6. Linkers may also be repeating units of alkyloxy (e.g. polyethylenoxy, PEG, polymethyleneoxy) and alkylamino (e.g. polyethyleneamino, Jeffamine™); and diacid ester and amides including succinate, succinamide, diglycolate, malonate, and caproamide.

Specific embodiments of A⁵ include where M2 is 0, such as:

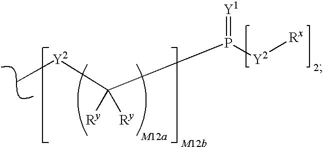

and where M12b is 1, Y¹ is oxygen, and Y^{2b} is independently oxygen (O) or nitrogen (N(R^{x})) such as:

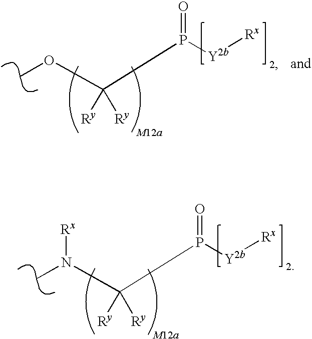

An embodiment of A⁵ includes:

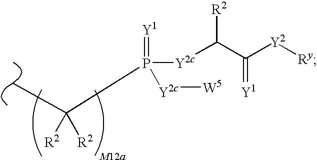

where W⁵ is a carbocycle such as phenyl or substituted phenyl, and Y^{2c} is independently O, N(R^{y}) or S. For example, R¹ may be H and n may be 1.

W⁵ also includes, but is not limited to, aryl and heteroaryl groups such as:

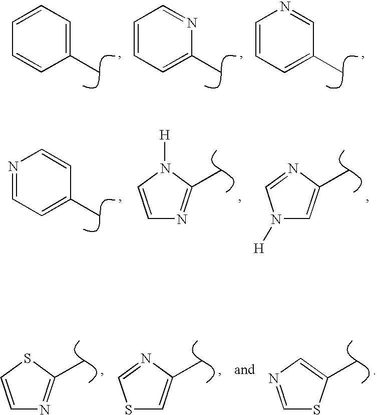

Another embodiment of A⁵ includes:

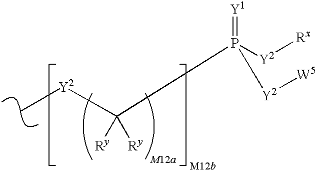

Such embodiments include:

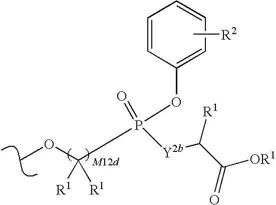

where Y^{2b} is O or N(R^{x}); M12d is 1, 2, 3, 4, 5, 6, 7 or 8; R¹ is H or C₁-C₆ alkyl; and the phenyl carbocycle is substituted with 0 to 3 R² groups where R² is C₁-C₆ alkyl or substituted alkyl. Such embodiments of A⁵ include phenyl phosphonamidate amino acid, e.g. alanate esters and phenyl phosphonate-lactate esters:

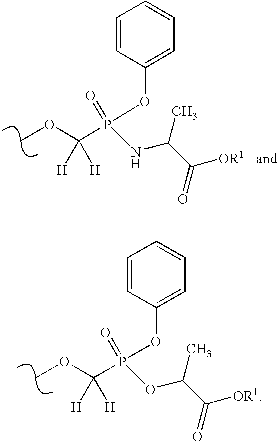

Embodiments of R^{x} include esters, carbamates, carbonates, thioesters, amides, thioamides, and urea groups:

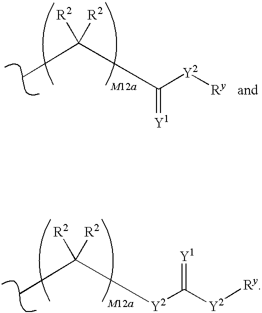

In one embodiment, the prodrug entity, PRD, is selected from the group consisting of C₁-C₆ alkoxycarbonyl, C₁-C₆ alkoxycarbonyloxymethylene, and C₃-C₇ cycloalkoxycarbonyloxymethylene.

In one embodiment, the prodrug entity, PRD is selected from the group consisting of isopropoxycarbonyl, cyclobutoxycarbonyloxymethylene, pent-3-oxycarbonyloxymethylene, cyclopentyloxycarbonyloxymethylene and isopropoxycarbonyloxymethylene.

In one embodiment, the prodrug entity, PRD, is selected from the group consisting of C₁-C₆ alkoxycarbonyl, C₁-C₆ alkoxycarbonyloxymethylene, and C₃-C₇ cycloalkoxycarbonyloxymethylene.

In one embodiment, the prodrug entity, PRD is selected from the group consisting of isopropoxycarbonyl, cyclobutoxycarbonyloxymethylene, pent-3-oxycarbonyloxymethylene, cyclopentyloxycarbonyloxymethylene and isopropoxycarbonyloxymethylene.

Compounds of the invention bearing one or more prodrug moieties may increase or optimize the bioavailability of the compounds as therapeutic agents. For example, bioavailability after oral administration may be beneficial and may depend on resistance to metabolic degradation in the gastrointestinal tract or circulatory system, and eventual uptake inside cells. Prodrug moieties are considered to confer said resistance by slowing certain hydrolytic or enzymatic metabolic processes. Lipophilic prodrug moieties may also increase active or passive transport of the compounds of the invention across cellular membranes (Darby, C. Antiviral Chem. & Chemotherapy (1995) Supp. 1, 6:54-63).

Exemplary embodiments of the invention includes phosphonamidate and phosphoramidate (collectively “amidate”) prodrug compounds. General formulas for phosphonamidate and phosphoramidate prodrug moieties include:

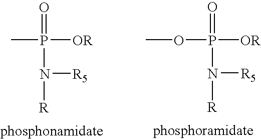

The phosphorus atom of the phosphonamidate group is bonded to a carbon atom of a compound of formula I, II, or III. The nitrogen substituent R₅ may include an ester, an amide, or a carbamate functional group. For example, R₅ may be —CR₂C(═O)OR′ where R′ is H, C₁-C₆ alkyl, C₁-C₆, substituted alkyl, C₆-C₂₀ aryl, C₆-C₂₀ substituted aryl, C₂-C₂₀ heteroaryl, or C₂-C₂₀ substituted heteroaryl.

Exemplary embodiments of phosphonamidate and phosphoramidate prodrugs include:

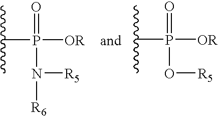

wherein R₅ is CR₂CO₂R₇ where R₆ and R₇ are independently H or C₁-C₈ alkyl.

The nitrogen atom may comprise an amino acid residue within the prodrug moiety, such as a glycine, alanine, or valine ester (e.g. valacyclovir, see: Beauchamp, et al Antiviral Chem. Chemotherapy (1992) 3:157-164), such as the general structure:

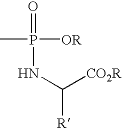

where R′ is the amino acid side-chain, e.g. H, CH₃, CH(CH₃)₂, etc.

An exemplary embodiment of a phosphonamidate prodrug moiety is:

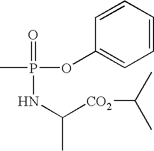

Specific values listed herein for radicals, substituents, and ranges, are for illustration only; they do not exclude other defined values or other values within defined ranges for the radicals and substituents.

A specific value for A² is N.

A specific value for A² is CRₐ.

A specific value for A³ is N.

A specific value for A³ is CRₐ;

A specific value for R¹ is H.

A specific value for R¹ is R_{b}

A specific value for R¹ is -Q-R_{c}.

A specific value for R¹ is methyl.

A specific value for R² is C₁-C₆alkoxycarbonyl, —C(═O)C(═O)ORₐ, or —C(═O)NRₐR_{g}.

A specific value for R² is C₁-C₆alkyl that is substituted with one or more groups independently selected from heterocycle, substituted heterocycle, —C(O)ORₐ, —C(═N—ORₐ)—NRₑRₑ, —C(═NRₐ)—NRₑRₑ, —P(═O)(Rₙ)(Rₙ), —C(═O)N(Rₑ)NRₑRₑ, —C(═O)NRₐR_{g}, and —C(═O)Rₕ.

A specific value for R² is C₃-C₈carbocycle that is substituted with one or more groups independently selected from heterocycle, —C(═O)ORₐ, —C(═O)NRₑRₑ, —C(═O)N(Rₐ)—S(O)₂Rₐ,

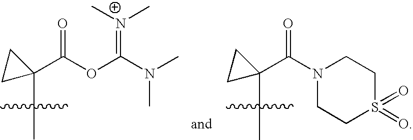

A specific value for R² is methoxycarbonyl, oxalo, N-(1-carboxycyclopropyl)aminocarbonyl, N-(2-carboxy-2-methylethyl)aminocarbonyl, or methylsulfonylaminocarbonyl.

A specific value for R² is N-(1-carboxycyclopropyl)aminocarbonylmethyl, N-(2-carboxy-2-methylethyl)aminocarbonylmethyl, tetrazoylmethyl, methylsulfonylaminocarbonylmethyl, methoxycarbonylmethyl, diethylphosphonylmethyl, 2,6-difluorobenzylphosphinylmethyl, 1,1-dioxothiomorpholinocarbonylmethyl, N-(2-aminosulfonylethyl)aminocarbonylmethyl, 5-methyl, 1,3,4-oxadiazolylmethyl, 5-(1-methylamino)-1,3,4-oxadiazolylmethyl, hydrazinocarbonylmethyl, imidazolyl, 5-amino-1,3,4-oxadiazolylmethyl, tetrazolylaminocarbonylmethyl, 1-benzimidazolylmethyl, 1,2,4-triazolylmethyl, 1-methyltetrazolylmethyl, 2-methyl-1,3,4-thiadiazolylmethyl, 2-methylimidazolylmethyl, 5-methyl-1,2,4-triazolylmethyl, 3-methyl-1,2,4-triazolylmethyl, 5-methyl-1,2,4-oxadiazolylmethyl, hydroxyamidinomethyl, amidinomethyl, 1,2-dihydro-5-oxa-1,3,4-oxadiazolylmethyl, 2-propynylaminocarbonylmethyl, 5-methyl-1,3-oxadiazolylmethyl, 2-benzimidazolylmethyl, N-(1,3,4-thiadiazol-2-yl)aminocarbonylmethyl, N-(5-pyrazolyl)aminocarbonylmethyl

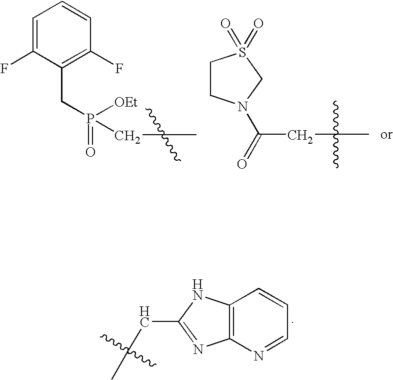

A specific value for R² is cyclopropyl, 1-carboxycyclopropyl, 1-methoxycarbonylcyclopropyl, 1-(aminocarbonyl)cyclopropyl, 1-(methylsulfonulaminocarbonyl)cycloprppyl, 1-(N-methylaminocarbonyl)cyclopropyl, 1-N-(2-hydroxy-1,1dimethylethyl)aminocarbonyl)cyclopropyl, 1-(N-(2-hydroxyethyl)aminocarbonyl)cyclopropyl,

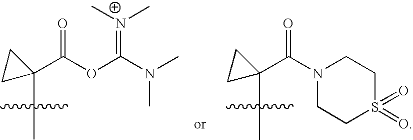

A specific value for R³ is H.

A specific value for R³ is halo.

A specific value for R³ is C₁-C₆alkyl that is optionally substituted with Rₖ.

A specific value for R³ is C₁-C₆alkyl that is substituted with Rₖ.

A specific value for R³ is 4-fluorobenzyl, or 4,6-difluoro-3-chlorobenzyl.

A specific value for R⁴ is halo.

A specific value for R⁴ is C₁-C₆alkyl that is optionally substituted with Rₖ.

A specific value for R⁴ is hydrogen.

A specific value for Z is two hydrogens.

A specific compound of the invention is

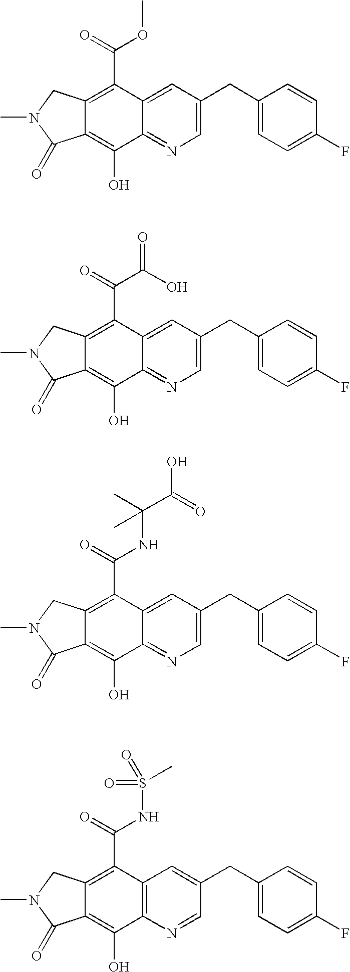

or a pharmaceutically acceptable salt or prodrug thereof.

A specific compound of the invention is

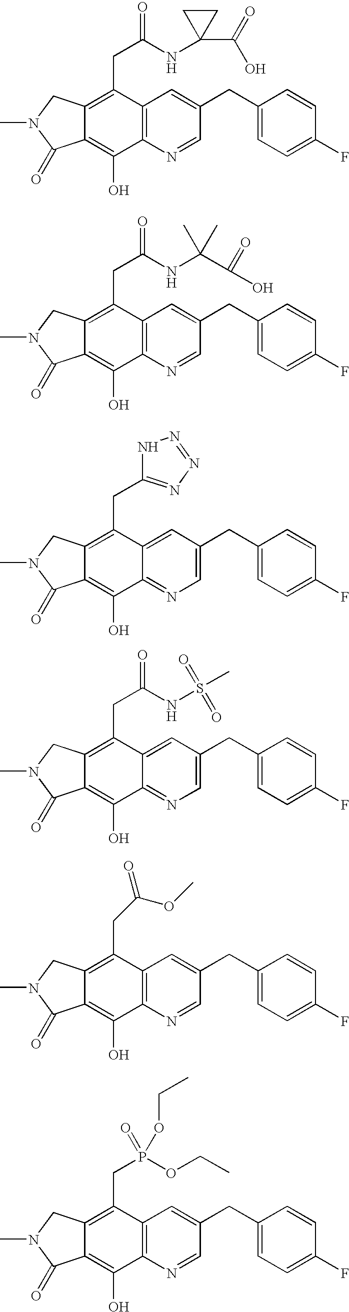

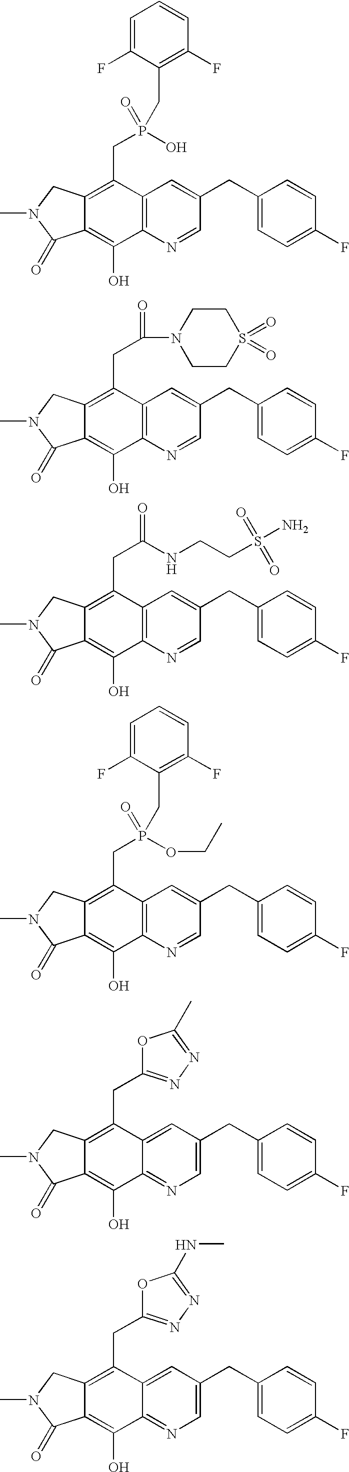

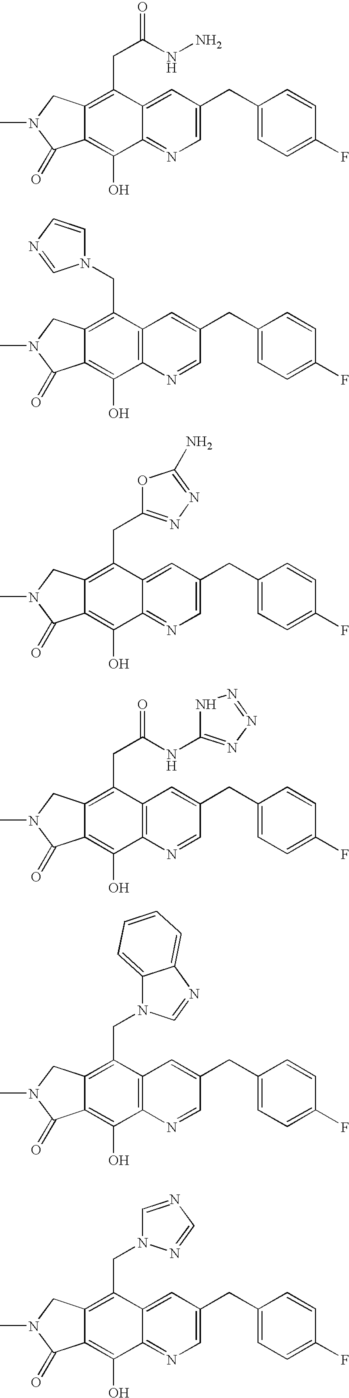

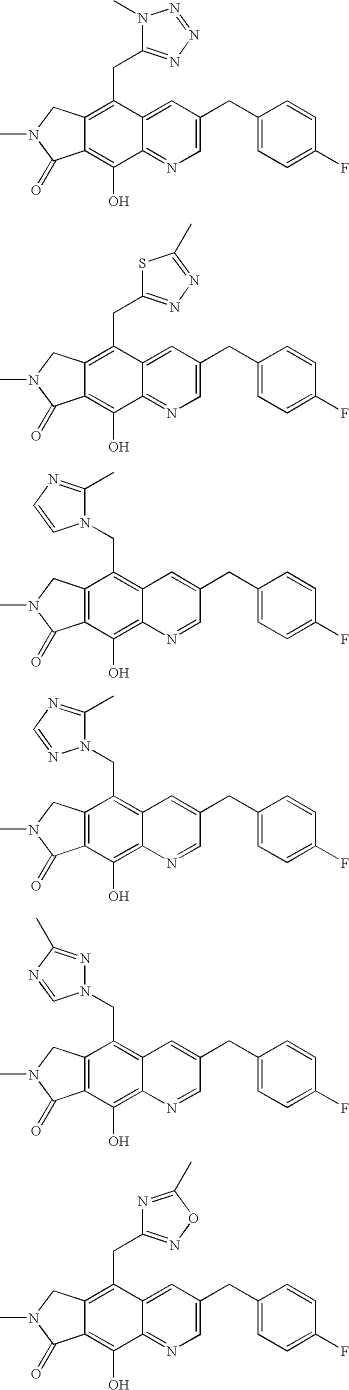

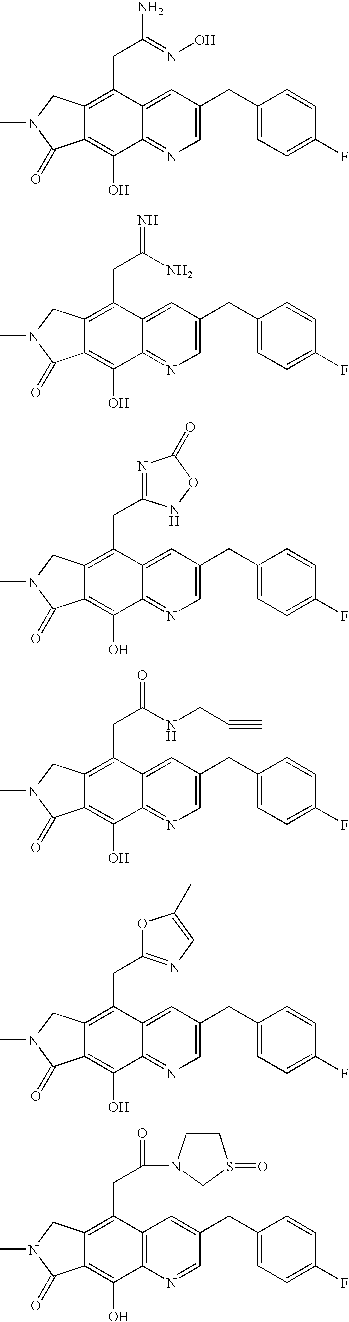

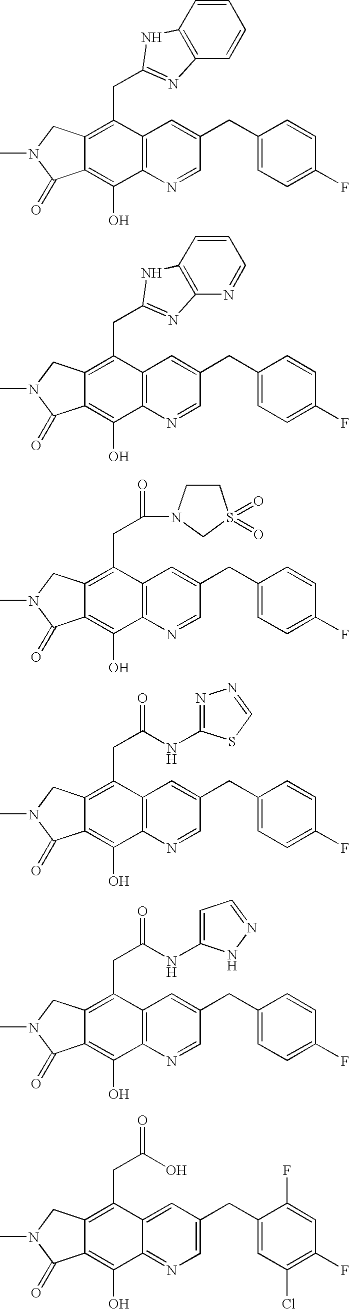

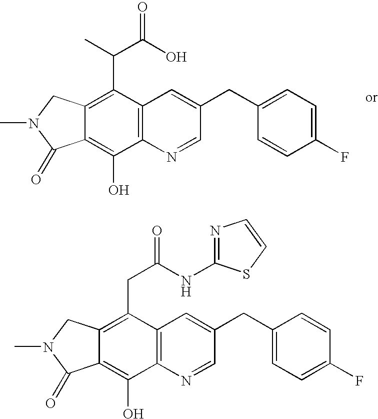

or a pharmaceutically acceptable salt or prodrug thereof.

A specific compound of the invention is

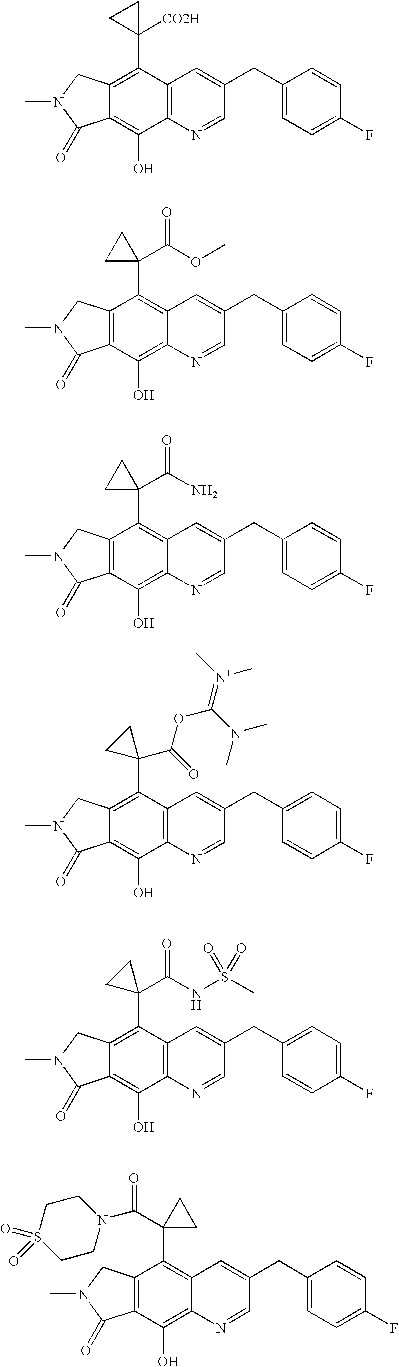

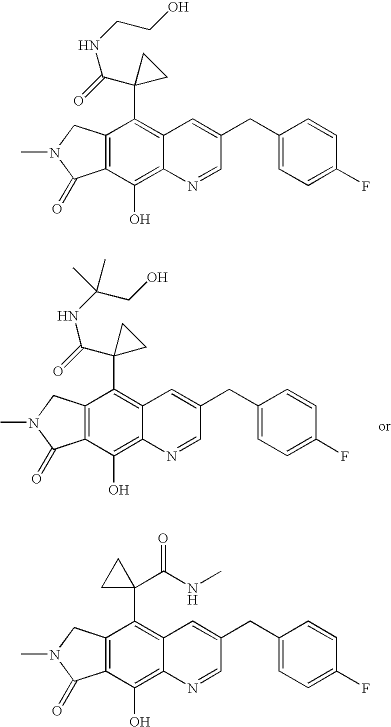

or a pharmaceutically acceptable salt or prodrug thereof.

A specific compound of the invention is

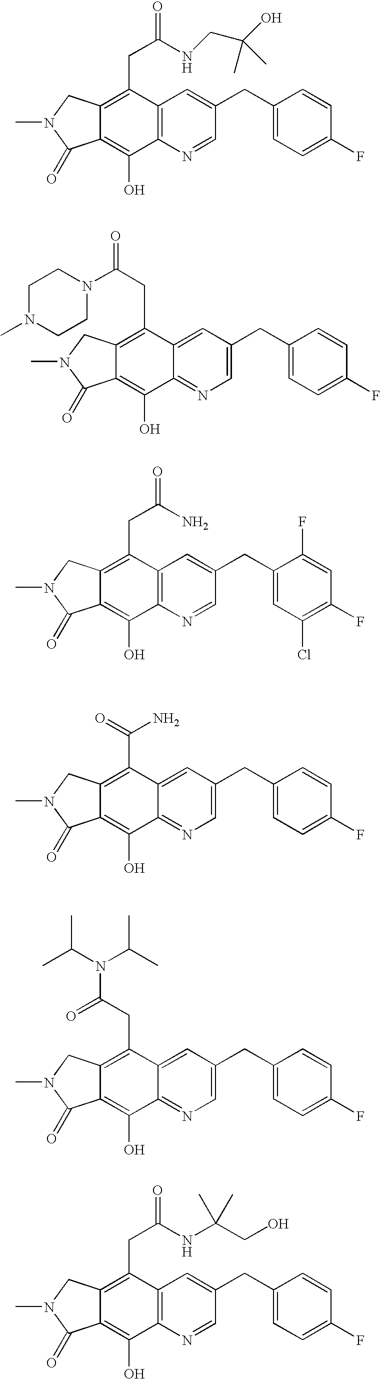

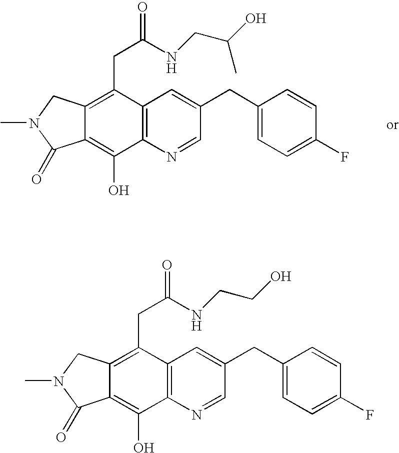

or a pharmaceutically acceptable salt or prodrug thereof.

Another embodiment of the invention is directed toward an HIV integrase inhibitor tricyclic compound of the invention which is capable of accumulating in human PBMC (peripheral blood mononuclear cells). PBMC refer to blood cells having round lymphocytes and monocytes. Physiologically, PBMC are critical components of the mechanism against infection. PBMC may be isolated from heparinized whole blood of normal healthy donors or buffy coats, by standard density gradient centrifugation and harvested from the interface, washed (e.g. phosphate-buffered saline) and stored in freezing medium. PBMC may be cultured in multi-well plates. At various times of culture, supernatant may be either removed for assessment, or cells may be harvested and analyzed (Smith R. et al (2003) Blood 102(7):2532-2540). The compounds of this embodiment may further comprise a phosphonate or phosphonate prodrug. Typically, the phosphonate or phosphonate prodrug has the structure A⁵ as described herein.

Optionally, the compounds of this embodiment demonstrate improved intracellular half-life of the compounds or intracellular metabolites of the compounds in human PBMC when compared to analogs of the compounds not having the phosphonate or phosphonate prodrug. Typically, the half-life is improved by at least about 50%, more typically at least in the range 50-100%, still more typically at least about 100%, more typically yet greater than about 100%.

In another embodiment, the intracellular half-life of a metabolite of the compound in human PBMCs is improved when compared to an analog of the compound not having the phosphonate or phosphonate prodrug. In such embodiments, the metabolite may be generated intracellularly, or it is generated within human PBMC. The metabolite may be a product of the cleavage of a phosphonate prodrug within human PBMCs. The phosphonate prodrug may be cleaved to form a metabolite having at least one negative charge at physiological pH. The phosphonate prodrug may be enzymatically cleaved within human PBMC to form a phosphonate having at least one active hydrogen atom of the form P—OH.

Those of skill in the art will also recognize that the compounds of the invention may exist in many different protonation states, depending on, among other things, the pH of their environment. While the structural formulae provided herein depict the compounds in only one of several possible protonation states, it will be understood that these structures are illustrative only, and that the invention is not limited to any particular protonation state—any and all protonated forms of the compounds are intended to fall within the scope of the invention.

The compounds of this invention optionally comprise salts of the compounds herein, especially pharmaceutically acceptable non-toxic salts containing, for example, Na⁺, Li⁺, K⁺, Ca⁺² and Mg⁺². Such salts may include those derived by combination of appropriate cations such as alkali and alkaline earth metal ions or ammonium and quaternary amino ions with an acid anion moiety, typically a carboxylic acid. The compounds of the invention may bear multiple positive or negative charges. The net charge of the compounds of the invention may be either positive or negative. Any associated counter ions are typically dictated by the synthesis and/or isolation methods by which the compounds are obtained. Typical counter ions include, but are not limited to ammonium, sodium, potassium, lithium, halides, acetate, trifluoroacetate, etc., and mixtures thereof. It will be understood that the identity of any associated counter ion is not a critical feature of the invention, and that the invention encompasses the compounds in association with any type of counter ion. Moreover, as the compounds can exists in a variety of different forms, the invention is intended to encompass not only forms of the compounds that are in association with counter ions (e.g., dry salts), but also forms that are not in association with counter ions (e.g., aqueous or organic solutions).

Metal salts typically are prepared by reacting the metal hydroxide with a compound of this invention. Examples of metal salts which are prepared in this way are salts containing Li⁺, Na⁺, and K⁺. A less soluble metal salt can be precipitated from the solution of a more soluble salt by addition of the suitable metal compound. In addition, salts may be formed from acid addition of certain organic and inorganic acids, e.g., HCl, HBr, H₂SO₄, H₃PO₄ or organic sulfonic acids, to basic centers, typically amines, or to acidic groups. Finally, it is to be understood that the compositions herein comprise compounds of the invention in their unionized, as well as zwitterionic form, and combinations with stoichiometric amounts of water as in hydrates.

Also included within the scope of this invention are the salts of the parental compounds with one or more amino acids, especially the naturally-occurring amino acids found as protein components. The amino acid typically is one bearing a side chain with a basic or acidic group, e.g., lysine, arginine or glutamic acid, or a neutral group such as glycine, serine, threonine, alanine, isoleucine, or leucine.

The compounds of the invention can also exist as tautomeric, resonance isomers in certain cases. Typically, the structures shown herein exemplify only one tautomeric or resonance form of the compounds. For example, hydrazine, oxime, hydrazone groups may be shown in either the syn or anti configurations. The corresponding alternative configuration is contemplated as well. All possible tautomeric and resonance forms are within the scope of the invention.

One enantiomer of a compound of the invention can be separated substantially free of its opposing enantiomer by a method such as formation of diastereomers using optically active resolving agents (Stereochemistry of Carbon Compounds (1962) by E. L. Eliel, McGraw Hill; Lochmuller, C. H., (1975) J. Chromatogr., 113:(3) 283-302). Separation of diastereomers formed from the racemic mixture can be accomplished by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure enantiomers. Alternatively, enantiomers can be separated directly under chiral conditions, method (3).

Under method (1), diastereomeric salts can be formed by reaction of enantiomerically pure chiral bases such as brucine, quinine, ephedrine, strychnine, α-methyl-β-phenylethylamine (amphetamine), and the like with asymmetric compounds bearing acidic functionality, such as carboxylic acid and sulfonic acid. The diastereomeric salts may be induced to separate by fractional crystallization or ionic chromatography. For separation of the optical isomers of amino compounds, addition of chiral carboxylic or sulfonic acids, such as camphorsulfonic acid, tartaric acid, mandelic acid, or lactic acid can result in formation of the diastereomeric salts.

Alternatively, by method (2), the substrate to be resolved may be reacted with one enantiomer of a chiral compound to form a diastereomeric pair (Eliel, E. and Wilen, S. (1994) Stereochemistry of Organic Compounds, John Wiley & Sons, Inc., p. 322). Diastereomeric compounds can be formed by reacting asymmetric compounds with enantiomerically pure chiral derivatizing reagents, such as menthyl derivatives, followed by separation of the diastereomers and hydrolysis to yield the free, enantiomerically enriched xanthene. A method of determining optical purity involves making chiral esters, such as a menthyl ester or Mosher ester, α-methoxy-α-(trifluoromethyl)phenyl acetate (Jacob III. (1982) J. Org. Chem. 47:4165), of the racemic mixture, and analyzing the NMR spectrum for the presence of the two atropisomeric diastereomers. Stable diastereomers can be separated and isolated by normal- and reverse-phase chromatography following methods for separation of atropisomeric naphthyl-isoquinolines (Hoye, T., WO 96/15111).

By method (3), a racemic mixture of two asymmetric enantiomers can be separated by chromatography using a chiral stationary phase (Chiral Liquid Chromatography (1989) W. J. Lough, Ed. Chapman and Hall, New York; Okamoto, (1990) “Optical resolution of dihydropyridine enantiomers by High-performance liquid chromatography using phenylcarbamates of polysaccharides as a chiral stationary phase”, J. of Chromatogr. 513:375-378).

Enantiomers can be distinguished by methods used to distinguish other chiral molecules with asymmetric carbon atoms, such as optical rotation and circular dichroism.

Improving the delivery of drugs and other agents to target cells and tissues has been the focus of considerable research for many years. Though many attempts have been made to develop effective methods for importing biologically active molecules into cells, both in vivo and in vitro, none has proved to be entirely satisfactory. Optimizing the association of the inhibitory drug with its intracellular target, while minimizing intercellular redistribution of the drug, e.g. to neighboring cells, is often difficult or inefficient.

Most agents currently administered parenterally to a patient are not targeted, resulting in systemic delivery of the agent to cells and tissues of the body where it is unnecessary, and often undesirable. This may result in adverse drug side effects, and often limits the dose of a drug (e.g., cytotoxic agents and other anti-cancer or anti-viral drugs) that can be administered. By comparison, although oral administration of drugs is generally recognized as a convenient and economical method of administration, oral administration can result in either (a) uptake of the drug through the cellular and tissue barriers, e.g. blood/brain, epithelial, cell membrane, resulting in undesirable systemic distribution, or (b) temporary residence of the drug within the gastrointestinal tract. Accordingly, a major goal has been to develop methods for specifically targeting agents to cells and tissues. Benefits of such treatment include avoiding the general physiological effects of inappropriate delivery of such agents to other cells and tissues, such as uninfected cells. Intracellular targeting may be achieved by methods and compositions which allow accumulation or retention of biologically active agents inside cells.

### Preparation of Compounds of the Invention

The compounds of the invention may be prepared by a variety of synthetic routes and methods known to those skilled in the art. The invention also relates to methods of making the compounds of the invention. The compounds may be prepared by any of the applicable techniques of organic synthesis. For example, known techniques are elaborated in: “Compendium of Organic Synthetic Methods”, John Wiley & Sons, New York, Vol. 1, Ian T. Harrison and Shuyen Harrison, 1971; Vol. 2, Ian T. Harrison and Shuyen Harrison, 1974; Vol. 3, Louis S. Hegedus and Leroy Wade, 1977; Vol. 4, Leroy G. Wade, jr., 1980; Vol. 5, Leroy G. Wade, Jr., 1984; and Vol. 6, Michael B. Smith; as well as March, J., “Advanced Organic Chemistry”, Third Edition, John Wiley & Sons, New York, 1985; “Comprehensive Organic Synthesis. Selectivity, Strategy & Efficiency in Modern Organic Chemistry” (9 Volume set) Barry M. Trost, Editor-in-Chief, Pergamon Press, New York, 1993.

A number of exemplary methods for the preparation of the compounds of the invention are provided herein. These methods are intended to illustrate the nature of such preparations and are not intended to limit the scope of applicable methods.

Deliberate use may be made of protecting groups to mask reactive functionality and direct reactions regioselectively (Greene, et al (1991) “Protective Groups in Organic Synthesis”, 2nd Ed., John Wiley & Sons). For example, useful protecting groups for the 8-hydroxyl group and other hydroxyl substituents include methyl, MOM (methoxymethyl), trialkylsilyl, benzyl, benzoyl, trityl, and tetrahydropyranyl. Certain aryl positions may be blocked from substitution, such as the 2-position as fluorine.

### Protection of Reactive Substituents.

Depending on the reaction conditions employed, it may be necessary to protect certain reactive substituents from unwanted reactions by protection before the sequence described, and to deprotect the substituents afterwards, according to the knowledge of one skilled in the art. Protection and deprotection of functional groups are described, for example, in Protective Groups in Organic Synthesis, by T. W. Greene and P. G. M Wuts, Wiley, Second Edition 1990. Reactive substituents which may be protected are shown in the accompanying schemes as, for example, [OH], [SH], etc.

### Preparation of Carboalkoxy-Substituted Phosphonate Bisamidates, Monoamidates, Diesters and Monoesters.

A number of methods are available for the conversion of phosphonic acids into amidates and esters. In one group of methods, the phosphonic acid is either converted into an isolated activated intermediate such as a phosphoryl chloride, or the phosphonic acid is activated in situ for reaction with an amine or a hydroxy compound.

The conversion of phosphonic acids into phosphoryl chlorides is accomplished by reaction with thionyl chloride, for example as described in J. Gen. Chem. USSR, 1983, 53, 480, Zh. Obschei Khim., 1958, 28, 1063, or J. Org. Chem., 1994, 59, 6144, or by reaction with oxalyl chloride, as described in J. Am. Chem. Soc., 1994, 116, 3251, or J. Org. Chem., 1994, 59, 6144, or by reaction with phosphorus pentachloride, as described in J. Org. Chem., 2001, 66, 329, or in J. Med. Chem., 1995, 38, 1372. The resultant phosphoryl chlorides are then reacted with amines or hydroxy compounds in the presence of a base to afford the amidate or ester products.

Phosphonic acids are converted into activated imidazolyl derivatives by reaction with carbonyl diimidazole, as described in J. Chem. Soc., Chem. Comm., 1991, 312, or Nucleosides Nucleotides 2000, 19, 1885. Activated sulfonyloxy derivatives are obtained by the reaction of phosphonic acids with trichloromethylsulfonyl chloride, as described in J. Med. Chem. 1995, 38, 4958, or with triisopropylbenzenesulfonyl chloride, as described in Tet. Lett., 1996, 7857, or Bioorg. Med. Chem. Lett., 1998, 8, 663. The activated sulfonyloxy derivatives are then reacted with amines or hydroxy compounds to afford amidates or esters.

Alternatively, the phosphonic acid and the amine or hydroxy reactant are combined in the presence of a diimide coupling agent. The preparation of phosphonic amidates and esters by means of coupling reactions in the presence of dicyclohexyl carbodiimide is described, for example, in J. Chem. Soc., Chem. Comm., 1991, 312, or J. Med. Chem., 1980, 23, 1299 or Coll. Czech. Chem. Comm., 1987, 52, 2792. The use of ethyl dimethylaminopropyl carbodiimide for activation and coupling of phosphonic acids is described in Tet. Lett., 2001, 42, 8841, or Nucleosides Nucleotides, 2000, 19, 1885.

A number of additional coupling reagents have been described for the preparation of amidates and esters from phosphonic acids. The agents include Aldrithiol-2, and PYBOP and BOP, as described in J. Org. Chem., 1995, 60, 5214, and J. Med. Chem., 1997, 40, 3842, mesitylene-2-sulfonyl-3-nitro-1,2,4-triazole (MSNT), as described in J. Med. Chem., 1996, 39, 4958, diphenylphosphoryl azide, as described in J. Org. Chem., 1984, 49, 1158, 1-(2,4,6-triisopropylbenzenesulfonyl-3-nitro-1,2,4-triazole (TPSNT) as described in Bioorg. Med. Chem. Lett., 1998, 8, 1013, bromotris(dimethylamino)phosphonium hexafluorophosphate (BroP), as described in Tet. Lett., 1996, 37, 3997, 2-chloro-5,5-dimethyl-2-oxo-1,3,2-dioxaphosphinane, as described in Nucleosides Nucleotides 1995, 14, 871, and diphenyl chlorophosphate, as described in J. Med. Chem., 1988, 31, 1305.

Phosphonic acids can be converted into amidates and esters by means of the Mitsonobu reaction, in which the phosphonic acid and the amine or hydroxy reactant are combined in the presence of a triaryl phosphine and a dialkyl azodicarboxylate. The procedure is described in Org. Lett., 2001, 3, 643, or J. Med. Chem., 1997, 40, 3842.

Phosphonic esters can also be obtained by the reaction between phosphonic acids and halo compounds, in the presence of a suitable base. The method is described, for example, in Anal. Chem., 1987, 59, 1056, or J. Chem. Soc. Perkin Trans., 1, 1993, 19, 2303, or J. Med. Chem., 1995, 38, 1372, or Tet. Lett., 2002, 43, 1161.

### Biological Activity of HIV-Integrase Inhibitor Compounds

Representative compounds of the invention were tested for biological activity by methods including anti-HIV assay, measuring inhibition of HIV-integrase strand transfer catalysis, and cytotoxicity. See: Wolfe, et al J. Virol. (1996) 70:1424-1432; Hazuda, et al Nucleic Acids Res. (1994) 22:1121-22; Hazuda, et al J. Virol. (1997) 71:7005-7011; Hazuda, et al Drug Design and Discovery (1997) 15:17-24; and Hazuda, et al Science (2000) 287:646-650. The antiviral activity of a compound of the invention can be determined using pharmacological models which are well known in the art. While many of the compounds of the present invention demonstrate inhibition of integration of HIV reverse-transcribed DNA, there may be other mechanisms of action whereby HIV replication or proliferation is affected. The compounds of the invention may be active via inhibition of HIV-integrase or other enzymes associated with HIV infection, AIDS, or ARC. Furthermore, the compounds of the invention may have significant activity against other viral diseases. Thus, the specific assays embodied herein are not intended to limit the present invention to a specific mechanism of action.

### HIV Integrase Assay (IC₅₀ Determination)

The HIV Integrase assay is carried out in Reacti-Bind High Binding Capacity Streptavidin coated plates (Pierce # 15502) in 100 μL reactions. The wells of the plate are rinsed once with PBS. Each well is then coated at room temperature for 1 h with 100 μL of 0.14 μM Donor DNA with the following sequence:

5′Biotin-ACC CTT TTA GTC AGT GTG GAA AAT CTC TAG
CAG T-3′
3′-GAA AAT CAG TCA CAC CTT TTA GAG ATC GTC A-5′

After coating, the plate was washed twice with PBS. 3′processing of the Donor DNA is started by adding 80 μL of Integrase/buffer mixture (25 mM HEPES, pH 7.3, 12.5 mM DTT, 93.75 mM NaCl, 12.5 mM MgCl₂, 1.25% Glycerol, 0.3125 uM integrase) to each well. 3′processing is allowed to proceed for 30 min at 37° C., after which, 10 μL of test compound and 10 μL of 2.5 uM DIG-labeled Target DNA with the following sequence:

5′-TGA CCA AGG GCT AAT TCA CT-3′DIG
3′DIG-ACT GGT TCC CGA TTA AGT GA-5′

are added to each well to allow strand transfer to proceed for 30 min at 37° C. The plate is then washed three times with 2×SSC for 5 min and rinsed once with PBS. For detection of integrated product, 100 μL of a 1/2000 dilution of HRP-conjugated anti-DIG antibody (Pierce #31468) are added to each well and incubated for 1 hour. The plate was then washed three times for 5 min each, with 0.05% Tween-20 in PBS. For signal development and amplification, 100 μL of SuperSignal ELISA Femto Substrate (Pierce #37075) are added to each well. Chemiluminescence (in relative light units) is read immediately at 425 nm in the SPECTRAmax GEMINI Microplate Spectrophotometer using the end point mode at 5 sec per well.

For IC₅₀ determinations, eight concentrations of test compounds in a 1/2.2 dilution series are used.

### Antiviral Assay in MT2 and MT4 Cells

For the antiviral assay utilizing MT-2 cells, 50 μL of 2× test concentration of 5-fold serially diluted compound in culture medium with 10% FBS was added to each well of a 96-well plate (9 concentrations) in triplicate. MT-2 cells were infected with HIV-IIIb at a multiplicity of infection (m.o.i) of 0.01 for 3 hours. Fifty microliters of infected cell suspension in culture medium with 10% FBS (˜1.5×10⁴ cells) was then added to each well containing 50 μL of diluted compound. The plates were then incubated at 37° C. for 5 days. For the antiviral assay utilizing MT-4 cells, 20 μL of 2× test concentration of 5-fold serially diluted compound in culture medium with 10% FBS was added to each well of a 384-well plate (7 concentrations) in triplicate. MT-4 cells were next mixed with HIV-IIIb at an m.o.i. of 0.1 and 20 μL of virus/cell mixture (˜2000 cells) was immediately added to each well containing 20 μL of diluted compound. The plates were then incubated at 37° C. for 5 days. After 5 days of incubation, 100 μL of CellTiter-Glo™ Reagent (catalog #G7571, Promega Biosciences, Inc., Madison, Wis.) was added to each well containing MT-2 cells and 40 μL to each well containing MT-4 cells. Cell lysis was carried out by incubating at room temperature for 10 min and then chemiluminescence was read.

### Cytotoxicity Assays in MT-2 and MT-4 Cells

For compound cytotoxicity assessment in MT-2 cells, the protocol was similar to that of the antiviral assay in MT-2 cells, except that uninfected cells and a 3-fold serial dilution of compounds were used. For cytotoxicity assessment in MT-4 cells, the protocol is similar to that of the antiviral assay in MT-4 cells, except that no virus was added.

Typically the compounds of the invention have an IC₅₀ of less than or equal to about 1 μM. Certain specific compounds of the invention have an IC₅₀ of less than or equal to about 60 nM, while other compounds have an IC₅₀ of less than or equal to about 25 nM. The compounds of the invention typically have an EC₅₀ of less than or equal to about 1 μM. Certain specific compounds of the invention have an EC₅₀ of less than or equal to about 60 nM, while other compounds of the invention have an IC₅₀ of less than or equal to about 25 nM. Certain compounds of the invention have an IC₅₀ of between >0 μM and about 1 μM, and an EC₅₀ of between >0 μM and about 1 μM. Other compounds of the invention have an IC₅₀ of between >0 μM and about 60 nM and an EC₅₀ of between >0 μM and about 60 nM. While other compounds of the invention have an IC₅₀ of between >0 μM and about 25 nM and an EC₅₀ of between >0 μM and about 25 nM.

### Pharmaceutical Formulations and Routes of Administration

Examples of pharmaceutically acceptable carriers and methods of manufacture for various compositions may be found in Remington's Pharmaceutical Sciences, 18^{th} Ed., Mack Publishing Co. (1990), which is incorporated in its entirety by reference herein.

The compounds of the invention may be formulated with conventional carriers, diluents and excipients, which will be selected in accord with ordinary practice. Tablets will contain excipients, glidants, fillers, binders, diluents and the like. Aqueous formulations are prepared in sterile form, and when intended for delivery by other than oral administration generally will be isotonic. Formulations optionally contain excipients such as those set forth in the “Handbook of Pharmaceutical Excipients” (1986) and include ascorbic acid and other antioxidants, chelating agents such as EDTA, carbohydrates such as dextrin, hydroxyalkylcellulose, hydroxyalkylmethylcellulose, stearic acid and the like.

Compounds of the invention and their physiologically acceptable salts (hereafter collectively referred to as the active ingredients) may be administered by any route appropriate to the condition to be treated, suitable routes including oral, rectal, nasal, topical (including ocular, buccal and sublingual), vaginal and parenteral (including subcutaneous, intramuscular, intravenous, intradermal, intrathecal and epidural). The preferred route of administration may vary with for example the condition of the recipient.

While it is possible for the active ingredients to be administered alone it is preferably to present them as pharmaceutical formulations. The formulations, both for veterinary and for human use, of the present invention comprise at least one active ingredient, as above defined, together with one or more pharmaceutically acceptable carriers (excipients, diluents, etc.) thereof and optionally other therapeutic ingredients. The carrier(s) must be “acceptable” in the sense of being compatible with the other ingredients of the formulation and not deleterious to the recipient thereof.

The formulations include those suitable for oral, rectal, nasal, topical (including buccal and sublingual), vaginal or parenteral (including subcutaneous, intramuscular, intravenous, intradermal, intrathecal and epidural) administration. The formulations may conveniently be presented in unit dosage form and may be prepared by any of the methods well known in the art of pharmacy. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Formulations of the present invention suitable for oral administration may be presented as discrete units such as capsules, cachets or tablets each containing a predetermined amount of the active ingredient; as a powder or granules; as solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein.

For infections of the eye or other external tissues e.g. mouth and skin, the formulations are preferably applied as a topical ointment or cream containing the active ingredient(s) in an amount of, for example, 0.075 to 20% w/w (including active ingredient(s) in a range between 0.1% and 20% in increments of 0.1% w/w such as 0.6% w/w, 0.7% w/w, etc), preferably 0.2 to 15% w/w and most preferably 0.5 to 10% w/w. When formulated in an ointment, the active ingredients may be employed with either a paraffinic or a water-miscible ointment base. Alternatively, the active ingredients may be formulated in a cream with an oil-in-water cream base.

If desired, the aqueous phase of the cream base may include, for example, at least 30% w/w of a polyhydric alcohol, i.e. an alcohol having two or more hydroxyl groups such as propylene glycol, butane 1,3-diol, mannitol, sorbitol, glycerol and polyethylene glycol (including PEG400) and mixtures thereof. The topical formulations may desirably include a compound which enhances absorption or penetration of the active ingredient through the skin or other affected areas. Examples of such dermal penetration enhancers include dimethylsulfoxide and related analogs.

The oily phase of the emulsions of this invention may be constituted from known ingredients in a known manner. While the phase may comprise merely an emulsifier (otherwise known as an emulgent), it desirably comprises a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations.

Emulgents and emulsion stabilizers suitable for use in the formulation of the present invention include Tween™ 60, Span™ 80, cetostearyl alcohol, benzyl alcohol, myristyl alcohol, glyceryl mono-stearate and sodium lauryl sulfate.

The choice of suitable oils or fats for the formulation is based on achieving the desired cosmetic properties, since the solubility of the active compound in most oils likely to be used in pharmaceutical emulsion formulations is very low. Thus the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for topical administration to the eye also include eye drops wherein the active ingredient is dissolved or suspended in a suitable carrier, especially an aqueous solvent for the active ingredient. The active ingredient is preferably present in such formulations in a concentration of 0.5 to 20%, advantageously 0.5 to 10% particularly about 1.5% w/w.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate.

Formulations suitable for nasal administration wherein the carrier is a solid include a coarse powder having a particle size for example in the range 20 to 500 microns (including particle sizes in a range between 20 and 500 microns in increments of 5 microns such as 30 microns, 35 microns, etc), which is administered in the manner in which snuff is taken, i.e. by rapid inhalation through the nasal passage from a container of the powder held close up to the nose. Suitable formulations wherein the carrier is a liquid, for administration as for example a nasal spray or as nasal drops, include aqueous or oily solutions of the active ingredient. Formulations suitable for aerosol administration may be prepared according to conventional methods and may be delivered with other therapeutic agents such as pentamidine for treatment of pneumocystis pneumonia.

Formulations suitable for vaginal administration may be presented as pessaries, tampons, creams, gels, pastes, foams or spray formulations containing in addition to the active ingredient such carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described. Preferred unit dosage formulations are those containing a daily dose or unit daily sub-dose, as herein above recited, or an appropriate fraction thereof, of an active ingredient.

It should be understood that in addition to the ingredients particularly mentioned above the formulations of this invention may include other agents conventional in the art having regard to the type of formulation in question, for example those suitable for oral administration may include flavoring agents.

The present invention further provides veterinary compositions comprising at least one active ingredient as above defined together with a veterinary carrier. Veterinary carriers are materials useful for the purpose of administering the composition and may be solid, liquid or gaseous materials which are otherwise inert or acceptable in the veterinary art and are compatible with the active ingredient. These veterinary compositions may be administered orally, parenterally or by any other desired route.

Compounds of the invention can be used to provide controlled release pharmaceutical formulations containing as active ingredient one or more compounds of the invention (“controlled release formulations”) in which the release of the active ingredient can be controlled and regulated to allow less frequency dosing or to improve the pharmacokinetic or toxicity profile of a given invention compound. Controlled release formulations adapted for oral administration in which discrete units comprising one or more compounds of the invention can be prepared according to conventional methods. Controlled release formulations may be employed for the treatment or prophylaxis of various microbial infections particularly human bacterial, human parasitic protozoan or human viral infections caused by microbial species including Plasmodium, Pneumocystis, herpes viruses (CMV, HSV 1, HSV 2, VZV, and the like), retroviruses, adenoviruses and the like. The controlled release formulations can be used to treat HIV infections and related conditions such as tuberculosis, malaria, pneumocystis pneumonia, CMV retinitis, AIDS, AIDS-related complex (ARC) and progressive generalized lymphadeopathy (PGL), and AIDS-related neurological conditions such as multiple sclerosis, and tropical spastic paraparesis. Other human retroviral infections that may be treated with the controlled release formulations according to the invention include Human T-cell Lymphotrophic virus (HTLV)-I and IV and HIV-2 infections. The invention accordingly provides pharmaceutical formulations for use in the treatment or prophylaxis of the above-mentioned human or veterinary conditions and microbial infections.

### Combination Therapy

The compounds of the invention may be employed in combination with other therapeutic agents for the treatment or prophylaxis of the infections or conditions indicated above. Examples of such further therapeutic agents include agents that are effective for the treatment or prophylaxis of viral, parasitic or bacterial infections or associated conditions or for treatment of tumors or related conditions include 3′-azido-3′-deoxythymidine (zidovudine, AXT), 2′-deoxy-3′-thiacytidine (3TC), 2′,3′-dideoxy-2′,3′-didehydroadenosine (D4A), 2′,3′-dideoxy-2′,3′-didehydrothymidine (D4T), carbovir (carbocyclic 2′,3′-dideoxy-2′,3′-didehydroguanosine), 3′-azido-2′,3′-dideoxyuridine, 5-fluorothymidine, (E)-5-(2-bromovinyl)-2′-deoxyuridine (BVDU), 2-chlorodeoxyadenosine, 2-deoxycoformycin, 5-fluorouracil, 5-fluorouridine, 5-fluoro-2′-deoxyuridine, 5-trifluoromethyl-2′-deoxyuridine, 6-azauridine, 5-fluoroorotic acid, methotrexate, triacetyluridine, 1-(2′-deoxy-2′-fluoro-1-β-arabinosyl)-5-iodocytidine (FIAC), tetrahydro-imidazo(4,5,1-jk)-(1,4)-benzodiazepin-2(1H)-thione (TIBO), 2′-nor-cyclicGMP, 6-methoxypurine arabinoside (ara-M), 6-methoxypurine arabinoside 2′-O-valerate, cytosine arabinoside (ara-C), 2′,3′-dideoxynucleosides such as 2′,3′-dideoxycytidine (ddC), 2′,3′-dideoxyadenosine (ddA) and 2′,3′-dideoxyinosine (ddI), acyclic nucleosides such as acyclovir, penciclovir, famciclovir, ganciclovir, HPMPC, PMEA, PMEG, PMPA, PMPDAP, FPMPA, HPMPA, HPMPDAP, (2R,5R)-9->tetrahydro-5-(phosphonomethoxy)-2-furanyladenine, (2R,5R)-1->tetrahydro-5-(phosphonomethoxy)-2-furanylthymine, other antivirals including ribavirin (adenine arabinoside), 2-thio-6-azauridine, tubercidin, aurintricarboxylic acid, 3-deazaneoplanocin, neoplanocin, rimantidine, adamantine, and foscarnet (trisodium phosphonoformate), antibacterial agents including bactericidal fluoroquinolones (ciprofloxacin, pefloxacin and the like), aminoglycoside bactericidal antibiotics (streptomycin, gentamicin, amicacin and the like) β-lactamase inhibitors (cephalosporins, penicillins and the like), other antibacterials including tetracycline, isoniazid, rifampin, cefoperazone, claithromycin and azithromycin, antiparasite or antifungal agents including pentamidine (1,5-bis(4′-aminophenoxy)pentane), 9-deaza-inosine, sulfamethoxazole, sulfadiazine, quinapyramine, quinine, fluconazole, ketoconazole, itraconazole, Amphotericin B, 5-fluorocytosine, clotrimazole, hexadecylphosphocholine and nystatin, renal excretion inhibitors such as probenicid, nucleoside transport inhibitors such as dipyridamole, dilazep and nitrobenzylthioinosine, immunomodulators such as FK506, cyclosporine A, thymosin α-1, cytokines including TNF and TGF-β, interferons including IFN-α, IFN-β, and IFN-γ, interleukins including various interleukins, macrophage/granulocyte colony stimulating factors including GM-CSF, G-CSF, M-CSF, cytokine antagonists including anti-TNF antibodies, anti-interleukin antibodies, soluble interleukin receptors, protein kinase C inhibitors and the like.

The compounds of the invention may be employed in combination with booster agents. One aspect of the invention provides the use of an effective amount of a booster agent to boost the pharmacokinetics of a compound of the invention. An effective amount of a booster agent, for example, the amount required to boost an HIV integrase inhibitor of the invention, is the amount necessary to improve the pharmacokinetic profile of the compound when compared to its profile when used alone. The inventive compound possesses a better efficacious pharmacokinetic profile than it would without the addition of the boosting agent. The amount of booster agent used to boost the integrase inhibitor potency of the inventive compound is, preferably, subtherapeutic (e.g., dosages below the amount of booster agent conventionally used for therapeutically treating HIV infection in a patient). A boosting dose for the compounds of the invention is subtherapeutic for treating HIV infection, yet high enough to effect modulation of the metabolism of the compounds of the invention, such that their exposure in a patient is boosted by increased bioavailability, increased blood levels, increased half life, increased time to peak plasma concentration, increased/faster inhibition of HIV integrase and/or reduced systematic clearance. An example of a boosting agent is Ritonavir® (ABBOTT Laboratories).

The compounds of the invention are preferably administered in an oral dosage form. The inventive compounds (or pharmaceutically acceptable salts thereof) are useful for the treatment of AIDS. The compounds (or pharmaceutically acceptable salts thereof) are useful for therapy. They are useful as a medicament. The compounds or pharmaceutically acceptable salts of the invention are useful in the manufacture of a medicament for the treatment of a viral infection (e.g. HIV). The pharmaceutical compositions of the invention may be used in the treatment of AIDS.

Still another aspect of this invention is to provide a kit for the treatment of disorders, symptoms and diseases where integrase inhibition plays a role, comprising two or more separate containers in a single package, wherein a compound, salt or composition of the invention is placed in combination with one or more of the following: a pharmaceutically acceptable carrier (excipient, diluent, etc.), a booster agent, and a therapeutically effective amount of another inventive compound, salt or composition thereof an AIDS treatment agent, such as an HIV inhibitor agent, an anti-infective agent or an immunomodulator agent.

The compounds can be made though a variety of synthetic routes. Generic procedures known in the art, such as those disclosed in WO/2004035577, which is hereby incorporated herein in its entirety, may be applied to synthesize a number of compounds of the invention.

The invention will now be illustrated by the following non-limiting Examples.

### EXAMPLES

### Example 1

### Preparation of Compound 3000

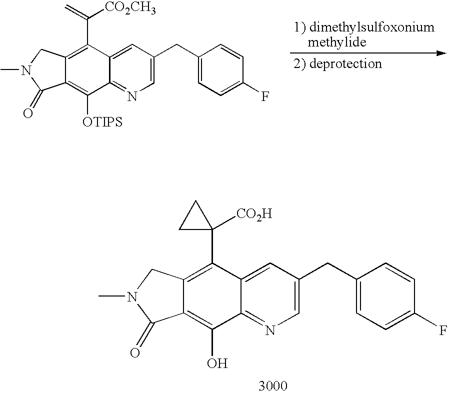

### Cyclopropyl Carboxylate 3000

To 140 mg (0.25 mmol) of the C5-acrylate, dissolved in 2 mL DMSO, and cooled to ice-bath temperature, is added 5 equivalents of freshly prepared dimethylsulfoxonium methylide in 2 mL DMSO. The reaction was stirred at rt and judged complete by LC/MS analysis after 30 minutes. The reaction mixture was diluted with 200 mL ethyl acetate, washed 3×100 mL saturated aq. Brine solution, dried over Na₂SO₄ and concentrated to give 160 mg of the intermediate cyclopropane, which was then carried through global deprotection by treatment LiOH in THF at 100° C. (microwave) for 4 h, followed by treatment with TFA in DCM to effect completion of TIPS-ether hydrolysis. The resulting carboxylate was purified by HPLC to give 32 mg of the cyclopropyl carboxylate 3000: ¹H NMR (300 MHz, CD₃OD) shows diagnostic peaks at δ (ppm): 8.85 (s, 1H), 8.61 (s, 1H), 7.28 (m, 2H), 7.04 (m, 2H), 4.82 (d, 1H), 4.55 (d, 1H) 4.32 (s, 2H), 3.21 (s, 3H), 1.96 (m, 1H), 1.75 (m, 1H), 1.45 (m, 1H), 1.18 (m, 1H). MS=407 (M+H).

### Example 2

### Preparation of Compound 133

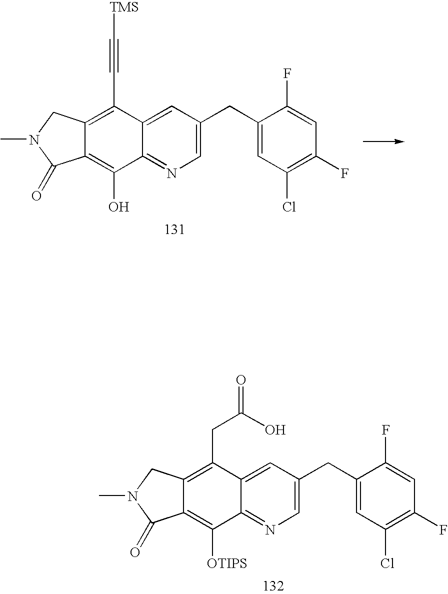

see J. Org. Chem., 1, 56, 1991, 3549 for precedent:

Acetylene 131 (190 g, 0.31 mmol, 1 equiv.) was prepared in a manner analogous to an example previously reported (April 2007, patent write up). It was stirred in THF (3 mL, 0.1 M) at 0° C. before freshly prepared dicyclohexylborane (3.5 mL, 6 equiv., 1 M see Organic Synthesis. coll. vol., 10, 2004, p. 273). The reaction was allowed to stir overnight. When the reaction was complete, MeOH (2 mL, 0.2 M) was added followed by NaOH (30 mg, 0.6 mmol, 2 equiv., dissolved in 3 mL water) and after 5 minutes H₂O₂ (0.5 mL, 1.28 mmol, 3 equiv., 30% in water). After the reaction was complete, it was stirred in 10% citric acid for 20 minutes along with ethyl acetate. The organic layer was washed with water, saturated NH₄Cl and brine. The solution was dried over sodium sulfate, filtered and concentrated in vacuo to yield acid 132. 300 MHz ¹H NMR ((DMSO-d₆) δ (ppm) 8.81 (s, 1H), 8.23 (s, 1H), 7.76-7.70 (m, 1H), 7.55-7.50 (m, 1H), 4.47 (s, 2), 4.23 (s, 2H), 3.92 (s, 2H), 3.05 (s, 3H), 1.47-1.40 (m, 1H), 1.03 (d, J=7.5 Hz, 18H). 300 MHz ¹⁹F NMR ((DMSO-d₆) δ (ppm) −114.24, 114.49. MS: 589.00 (M+1).

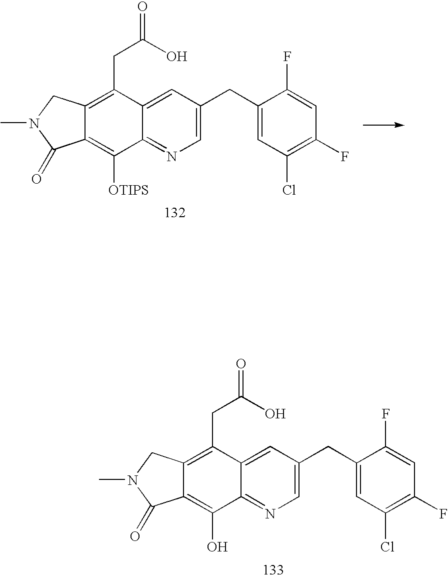

Compound 133 was made in a manner similar to an example shown above.

300 MHz ¹H NMR ((DMSO-d₆) δ (ppm) 8.83 (s, 1H), 8.34 (s, 1H), 7.76-7.70 (m, 1H), 7.55-7.50 (m, 1H), 4.49 (s, 2H), 4.24 (s, 2H), 3.05 (s, 3H).

MS: 433.19 (M+1).

### Example 3

### Preparation of Compound 17

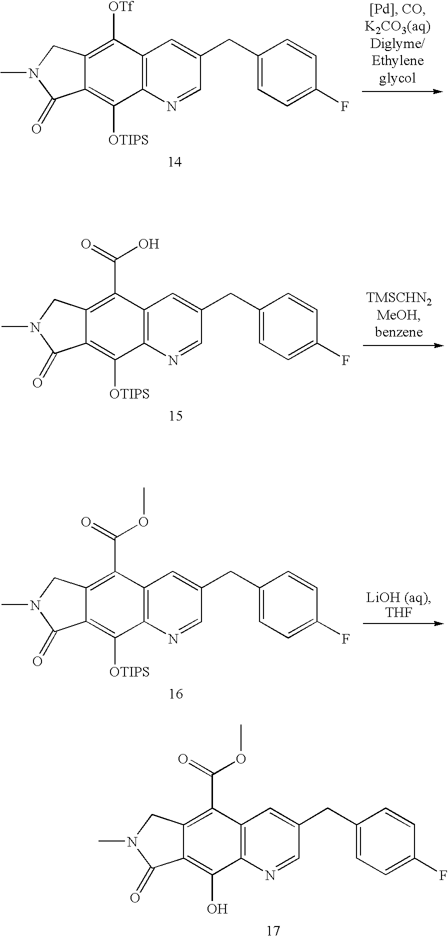

Compound 15: Procedure adapted from J. Comb. Chem. 2002, 4, 2, 109-111. To a solution of intermediate 14 [filed previously in 2007] (200 mg, 0.319 mmol) in toluene (3.19 mL) was added Hemmann's catalyst, trans-Di(mu-acetoato)bis[di-o-tolyl-phosphino)benzyl]dipalladium (II) (120 mg, 0.128 mmol) and BINAP (120 mg, 0.191 mmol). Subsequently, Diglyme (6.38 mL), ethylene glycol (0.64 mL) and K₂CO₃ (1.12 mL, 1M Aqueous solution) were sequentially added. The solution was degassed under high vacuum (5 minutes) and flushed with carbon monoxide from a balloon. The flushing was repeated several times. The mixture was heated at 90° C. under CO atmosphere for 1 hour then cooled down to room temperature. The reaction mixture was diluted with ethyl acetate then quenched with 5% Citric Acid solution. The layers were separated and the aqueous layer was extracted with ethyl acetate. The combined organic layer was washed with water (several times) and brine, then dried (over Na₂SO₄), filtered and concentrated in vacuo to afford product 15 (500 mg crude solid) with no further purification or characterization; MS: 523 (M+1).

Compound 16: To carboxylic acid 15 (300 mg crude, ˜0.1911 mmol) dissolved in benzene (1.6 mL) and methanol (0.440 mL) was added TMSCHN₂ (0.28 mL, 0.56 mmol, 2M diethyl ether solution) dropwise. The reaction was stirred at room temperature under nitrogen atmosphere for 30 minutes, at which point the reaction was complete. The volatiles were removed in vacuo and the crude residue was purified by ISCO flash column chromatography with 3/7 EtOAc/Hexanes to yield 16 (43 mg, 42%—2 steps, but contaminated with protonolysis by-product from carbonylation-18% of material by LC): 300 MHz ¹H NMR (CDCl₃) δ (ppm) 9.16 (s, 1H), 8.69 (s, 1H), 7.22 (m, 2H), 7.04 (m, 2H), 4.684 (s, 2H), 4.20 (s, 2H), 3.97 (s, 3H), 3.21 (s, 3H), 1.52 (m, 3H), 1.13 (d, 18H); MS: 537 (M+1).

Compound 17: To a solution of ester 16 (43 mg, 0.08 mmol) dissolved in THF (0.8 mL, 0.1M) was added DMAP (3 mg, 0.024 mmol) and a solution of LiOH*H2O (10 mg, 0.24 mmol) in water (0.4 mL). The reaction was stirred at room temperature for 3 hours at which point the reaction was diluted with ethyl acetate and water. The mixture was acidified with 1N HCl (to pH=2) and the product was extracted with ethyl acetate twice. The organic layer was washed with brine (×2) then dried (over Na₂SO₄), filtered and concentrated in vacuo. The residue was purified by reversed phase HPLC (Phenomenex Gemini Axia-packed column with 0.1% TFA in the mobile phase) to afford the desired product 17 (9.6 mg) as the TFA salt: 300 MHz ¹H NMR (CDCl₃) δ (ppm) 9.38 (s, 1H), 8.88 (s, 1H), 7.21 (m, 2H), 7.04 (m, 2H), 4.77 (s, 2H), 4.23 (s, 2H), 3.99 (s, 3H), 3.23 (s, 3H); 300 MHz ¹⁹F NMR (CDCl₃) δ (ppm) −76.38, −116.45; MS: 381 (M+1).

### Example 4

### Preparation of Compound 2998

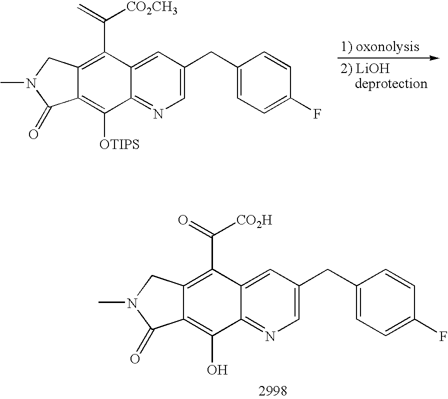

To 85 mg of the acrylate, (prepared as reported in an earlier patent filing), dissolved in 4 mL DCM, at dry-ice/acetone bath temperature, was introduced a stream of O₃. After 20 minutes, 200 μL methyl sulfide was added and the reaction allowed to warm to rt with stirring. 60 mg of the crude product was obtained upon concentration of the reaction mixture, and was submitted directly to treatment with LiOH at 130° C. (via microwave) in a 1:1 solution of THF/water. After 30 minutes, the reaction was quenched with 1 mL 1N HCl and the solution injected directly onto HPLC, to obtain 4 mg of the oxalate analog 2998: ¹H NMR (300 MHz, CD₃OD) shows diagnostic peaks at δ (ppm): 8.85 (m, 2H), 7.32 (m, 2H), 7.05 (m, 2H), 4.81 (s, 2H), 4.25 (s, 2H), 3.20 (s, 3H). MS=395 (M+H).

### Example 5

### Preparation of Compound 20

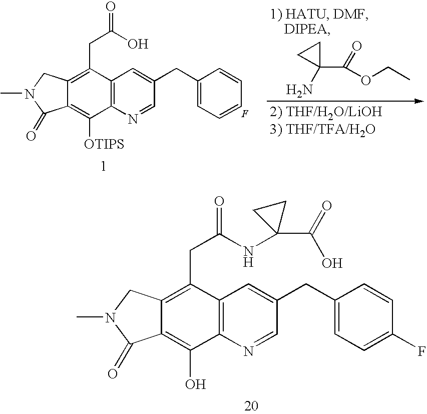

Compound 20 was prepared from compound 1 and purified by flash chromatography.

Purified compound was then dissolved in THF (10 mL, 0.01M) and to this was added H₂0 (1 mL), LiOH (1 mmol) and allowed to stir until ester cleaved via LCMS. When reaction was completed it was diluted with EtAc, and washed 2× with water. Aqueous layer was extracted 3× with EtAc and combined organic fractions are washed successively with water (2×), brine and then allowed to dry over Na₂SO₄ before filtering and concentrating in vacuo. TIPS group was deprotected with TFA and isolated product purified via HPLC

### Compound 20:1-{2-[3-(4-Fluoro-benzyl)-9-hydroxy-8-oxo-7,8-dihydro-6H-pyrrolo[3,4-g]quinolin-5-yl]-acetylamino}-cyclopropanecarboxylic acid

¹H-NMR (300 MHZ; DMSO-d₆): d 8.77 (s, 1H), 8.72 (s, 1H), 8.41 (s, 1H), 7.35 (t, 2H), 7.12 (t, 2H), 4.53 (s, 2H), 4.20 (s, 1H), 3.76 (s, 2H), 3.05 (s, 3H), 1.30 (br, 2H), 0.90 (br, 2H).

¹⁹F-NMR (300 MHZ; DMSO-d₆): −117.33, −74.72. MS [M+H]+=464.19

### Example 6

### Preparation of Compound 20

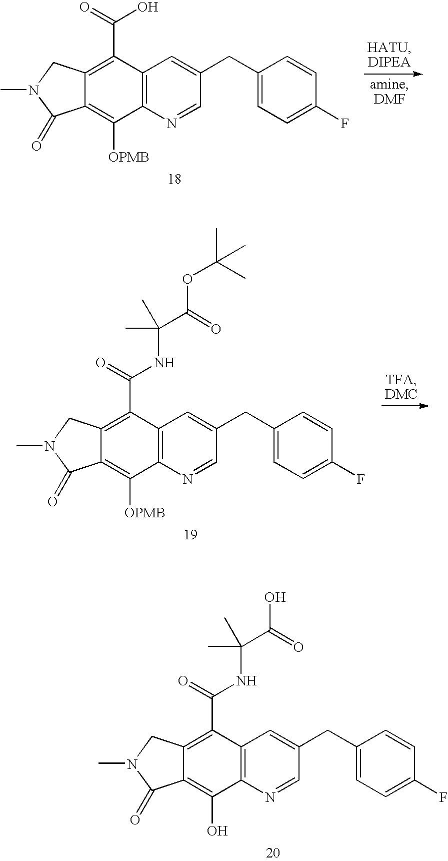

Compound 19: A solution of carboxylic acid 18 [filed previously in 2007] (20 mg, 0.0411 mmol) in DMF (0.5 mL) that had been stirred with HATU (23 mg, 0.0617 mmol) and DIPEA (0.036 mL, 0.206 mmol) for 5 minutes was treated with a-Aminoisobutyric acid t-Butyl ester hydrochloride salt (16.1 mg, 0.0822 mmol). The reaction mixture was stirred for 4 hours at room temperature, under nitrogen atmosphere, upon which diluted with ethyl acetate and quenched with water. The organic layer was washed with water, aqueous LiCl, and brine, then dried (NaSO₄), filtered and concentrated. The residue was purified by ISCO flash column chromatography with 4/1 EtOAc/Hexanes to afford the desired product 19 (15 mg, 60%): 300 MHz ¹H NMR (CDCl₃) δ (ppm) 8.65 (s, 1H), 8.38 (s, 1H), 7.43 (d, 2H), 7.26 (m, 2H), 7.02 (m, 2H), 6.73 (d, 2H), 5.53 (s, 2H), 4.41 (s, 2H), 4.13 (s, 2H), 3.74 (s, 3H), 2.93 (s, 3H), 1.59 (s, 6H), 1.53 (s, 9H); MS: 628 (M+1).

Compound 20: A solution of intermediate 19 (15 mg, 0.024 mmol) in dichloromethane (1 mL) was treated with Trifluoroacetic acid (0.25 mL). The reaction mixture was stirred at room temperature under an inert atmosphere for 1 hour upon which the mixture was azeotroped with toluene/THF repeatedly. The solid was triturated in ether/methanol (3/1) to afford the desired product 20 (7.2 mg, 67%) as the parent (white) solid: 300 MHz ¹H NMR (DMSO-d₆) δ (ppm) 8.87 (s, 1H), 8.27 (s, 1H), 7.34 (m, 2H), 7.14 (m, 2H), 4.51 (s, 2H), 4.19 (s, 2H), 3.04 (s, 3H), 1.43 (s, 6H); 300 MHz ¹⁹F NMR (DMSO-d₆) δ (ppm) −117.2; MS: 452 (M+1).

### Example 7

### Preparation of Compound 21

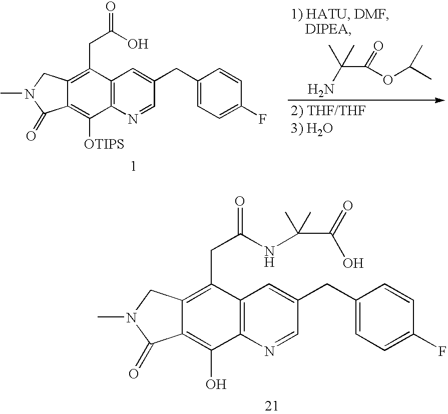

Compound 21 was prepared from compound 1 and purified by flash chromatography. Purified compound was then dissolved in THF (10 mL, 0.01M) and to this TFA (0.5 mL) was added and reaction stirred and monitored via LCMS. After t-BOC deprotection, 3 drops of water was added and TIPS removal monitored via LCMS. When completed reaction was concentrated in vacuo, triturated with toluene 3× and toluene removed in vacuo then triturated with 50 ml of 50:40:10 Et₂O:Hexanes:MeOH before filtering and allowing to air dry before purifying by HPLC. Compound 21: 2-{2-[3-(4-Fluoro-benzyl)-9-hydroxy-8-oxo-7,8-dihydro-6H-pyrrolo[3,4-g]quinolin-5-yl]-acetylamino}-2-methyl-propionic acid

¹H-NMR (300 MHZ; DMSO-d₆): d 8.77 (s, 1H), 8.47 (d, 1H), 7.36 (q, 2H), 7.12 (t, 2H), 4.52 (s, 2H), 4.20 (s, 1H), 3.79 (s, 2H), 3.05 (s, 3H), 1.30 (br, 2H), 0.90 (br, 2H).

¹⁹F-NMR (300 MHZ; DMSO-d₆): −117.33, −74.72. MS [M+H]+=466.17

### Example 8

### Preparation of Compound 126

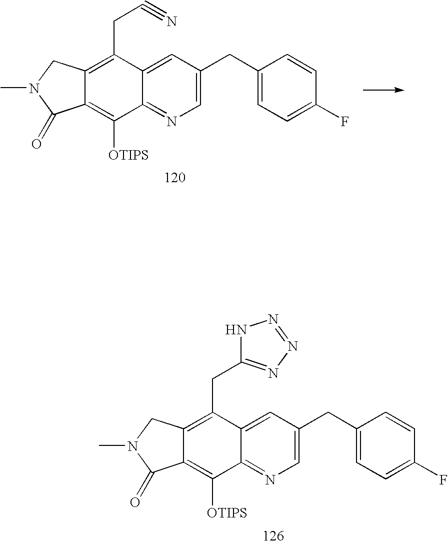

Into a microwave vial was added nitrile 120 (62 mg, 0.12 mmol, 1 equiv.) and THF (3 ml, 0.05 M) followed by tri-n-butyltin azide (160 mg, 0.47 mmol, 4 equiv) and di-n-butyltin oxide (6 mg, 0.03 mmol, 0.2 equiv). The flask was then subjected to heating at 130° C. via microwave for 2 hr. After the reaction was complete, it was diluted with ethyl acetate and quenched with water. The organic layer saturated was washed with NH₄Cl, aqueous LiCl, and brine, then dried (Na₂SO₄), filtered and concentrated down. It was washed with Hexanes/Diethyl ether and air dried. 400 MHz ¹H NMR (DMSO) δ (ppm): 8.78 (s, 1H), 8.38 (s, 1H), 7.39-7.34 (m, 2H), 7.15-7.05 (m, 2H), 4.63 (s, 2H), 4.55 (s, 2H), 4.16 (s, 2H), 3.06 (s, 3H), 1.49-1.52 (m, 3H), 1.12-1.15 (m, 9H). ¹⁹F NMR (CDCl₃) δ (ppm): −117.18. MS: 561.32 (M+1).

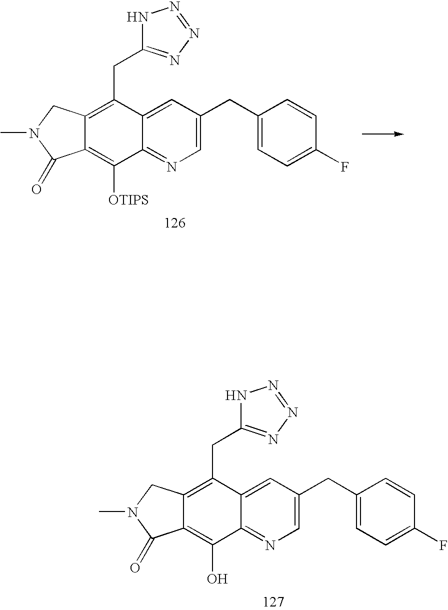

Compound 127 was made in a manner similar to an example shown above. 400 MHz ¹H NMR ((DMSO-d₆) δ (ppm): 8.79 (s, 1H), 8.39 (s, 1H), 7.36-7.31 (m, 2H), 7.13-7.07 (m, 2H), 4.62 (s, 2H), 4.57 (s, 2H), 4.17 (s, 2H), 3.05 (s, 3H).

¹⁹F NMR ((DMSO-d₆) δ (ppm): −74.93, −117.27. MS: 405.24 (M+1).

### Example 9

### Preparation of Compound 102

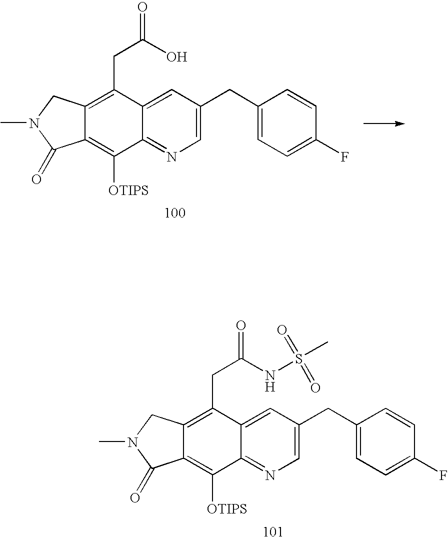

A solution of carboxylic acid 100 (250 mg, 0.46 mmol) in DMF (5 mL) was stirred with HATU (265 mg, 0.69 mmol, 1 equiv.) and DIPEA (290 μL, 1.63 mmol, 3.5 equiv.) for 5 minutes before being treated with methanesulfonamide (110 mg, 1.16 mmol, 2.5 equiv.) and allowed to stir at room temperature under an inert atmosphere. When the reaction was complete, it was diluted with ethyl acetate and quenched with water. The organic layer was washed with saturated NH₄Cl and brine, then dried (Na₂SO₄), filtered and concentrated. The residue was purified by chromatography on silica gel (0-5%—methanol/ethyl acetate) to afford the desired product 101. 300 MHz ¹H NMR (CDCl₃) δ (ppm): 11.04 (bs, 1H), 8.72 (s, 1H), 8.04 (s, 1H), 7.29-7.21 (m, 2H), 7.08-7.00 (m, 2H), 4.37 (s, 2H), 4.19 (s, 2H), 3.91 (s, 2H), 3.27 (s, 2H), 2.82 (s, 3H), 2.30 (m, 3H), 1.49-1.52 (m, 3H), 1.12-1.15 (m, 9H). ¹⁹F NMR (CDCl₃) δ (ppm): −116.66. MS: 614.33 (M+1).

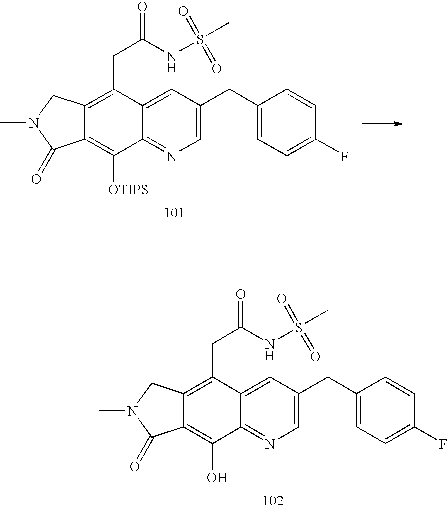

Compound 101 (155 mg, 0.25 mmol, 1 equiv) was stirred in THF (5 mL) and water (2 mL) before adding TFA (0.5 mL). The reaction was warmed to 50° C. After the reaction was complete, it was concentrated in vacuo and azeotroped with toluene. The solid was washed with Hexanes and ethyl ether before being air dried. 300 MHz ¹H NMR ((DMSO-d₆) δ (ppm) 12.10 (bs, 1H), 8.82 (s, 1H), 8.35 (s, 1H), 7.39-7.26 (m, 2H), 7.15-7.05 (m, 2H), 4.49 (s, 2H), 4.21 (s, 2H), 4.00 (s, 2H), 3.20 (s, 2H), 3.06 (s, 3H), 2.30 (m, 3H). 300 MHz ¹⁹F NMR ((DMSO-d₆) δ (ppm): −117.25. MS: 458.35 (M+1).

### Example 10

### Preparation of Compound 105

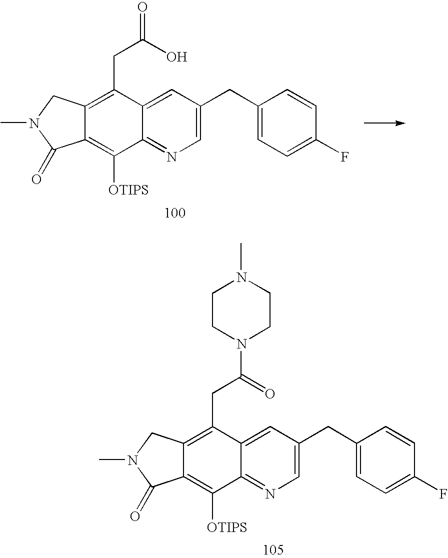

Compound 105 was made in a manner similar to Example 9. 300 MHz ¹H NMR (CDCl₃) δ (ppm): 8.69 (s, 1H), 7.76 (s, 1H), 7.29-7.21 (m, 2H), 7.08-7.00 (m, 2H), 4.35 (s, 2H), 4.17 (s, 2H), 3.83 (s, 2H), 3.68-3.62 (m, 2H), 3.60-3.55 (m, 2H), 3.18 (s, 3H), 2.33 (s, 3H), 2.37-2.30 (m, 4H), 1.49-1.52 (m, 3H), 1.12-1.15 (m, 9H). ¹⁹F NMR (CDCl₃) δ (ppm): −116.55. MS: 619.34 (M+1).

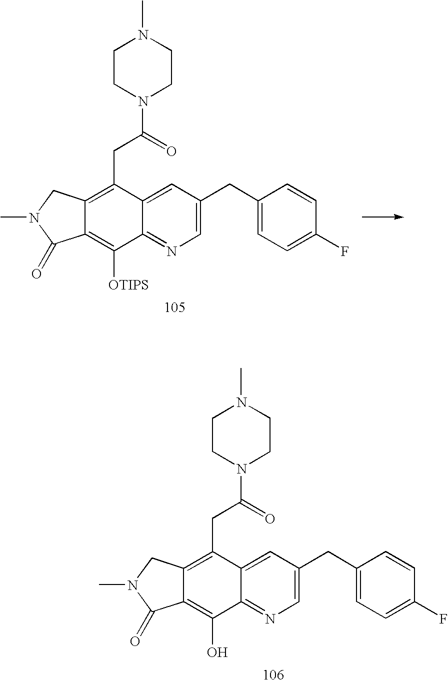

Compound 106 was made in a manner similar to Example 9. 300 MHz ¹H NMR ((DMSO-d₆) δ (ppm): 8.76 (s, 1H), 8.07 (s, 1H), 7.33-7.25 (m, 2H), 7.12-7.05 (m, 2H), 4.42 (s, 2H), 4.22 (s, 2H), 3.99 (s, 2H), 3.68-3.50 (m, 4H), 3.12 (s, 3H), 2.86 (s, 3H), 2.37-2.30 (m, 4H). ¹⁹F NMR ((DMSO-d₆) δ (ppm): −76.43, −118.79. MS: 463.20 (M+1).

### Example 11

### Preparation of Compound 3001

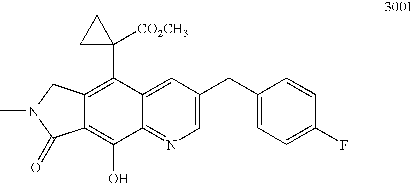

Cyclopropyl carboxyester 3001: Subsequent to the cyclopropanation of the acrylate as reported in Example 1, treatment of 20 mg of the crude intermediate with TFA in DCM effected TIPS-ether hydrolysis to provide, after HPLC purification, 3 mg methyl ester 3001: ¹H NMR (300 MHz, CD₃OD) shows diagnostic peaks at δ (ppm): 8.83 (s, 1H), 8.45 (s, 1H), 7.30 (m, 2H), 7.11 (m, 2H), 4.74 (d, 1H), 4.52 (d, 1H), 4.31 (s, 2H), 3.58 (s, 3H), 3.18 (s, 3H), 1.92 (m, 1H), 1.78 (m, 1H), 1.46 (m, 1H), 1.19 (m, 1H). MS=421 (M+H).

### Example 12

### Preparation of Compound 135

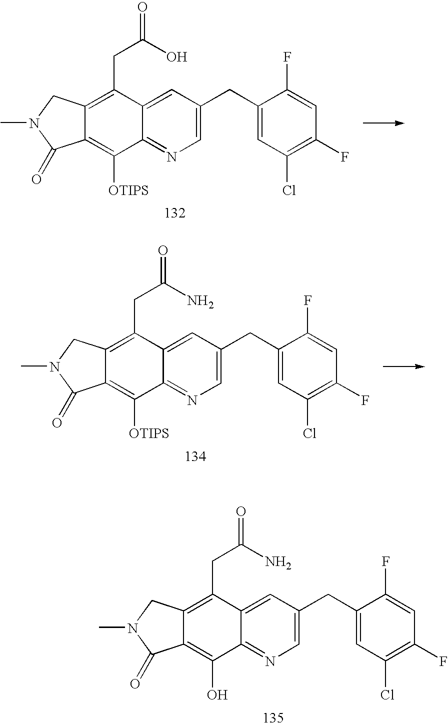

Compound 135 was prepared as illustrated above. 300 MHz ¹H NMR (acetone₆) δ (ppm) 8.78 (s, 1H), 8.44 (s, 1H), 7.32-7.28 (m, 1H), 7.09-7.04 (m, 1H), 4.52 (s, 2H), 4.63 (s, 2H), 4.35 (s, 2H), 3.85 (s, 2H), 2.99 (s, 3H). MS: 432.39 (M+1).

### Example 13

### Preparation of Compound 136

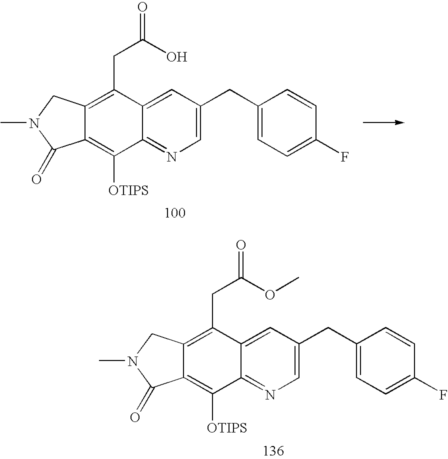

Compound 100 (200 mg, 0.37 mmol, 1 equiv.) was dissolved in benzene/MeOH (3 mL/1 mL) followed by addition of TMSCHN₂ (600 μL, 1.2 mmol, 3 equiv.). After the reaction was complete, it was concentrated in vacuo to yield compound 136 which was used as is. 300 MHz ¹H NMR (CDCl₃) δ (ppm) 8.69 (s, 1H), 8.06 (s, 1H), 7.41-7.30 (m, 2H), 7.16-7.01 (m, 2H), 4.46 (s, 2), 4.19 (s, 2H), 3.84 (s, 2H), 3.63 (s, 3H), 3.21 (s, 3H), 1.47-1.40 (m, 1H), 1.03 (d, J=7.5 Hz, 18H). 300 MHz ¹⁹F NMR (CDCl₃) δ (ppm) −117.24 MS: 551.32 (M+1).

### Example 14

### Preparation of Compound 22

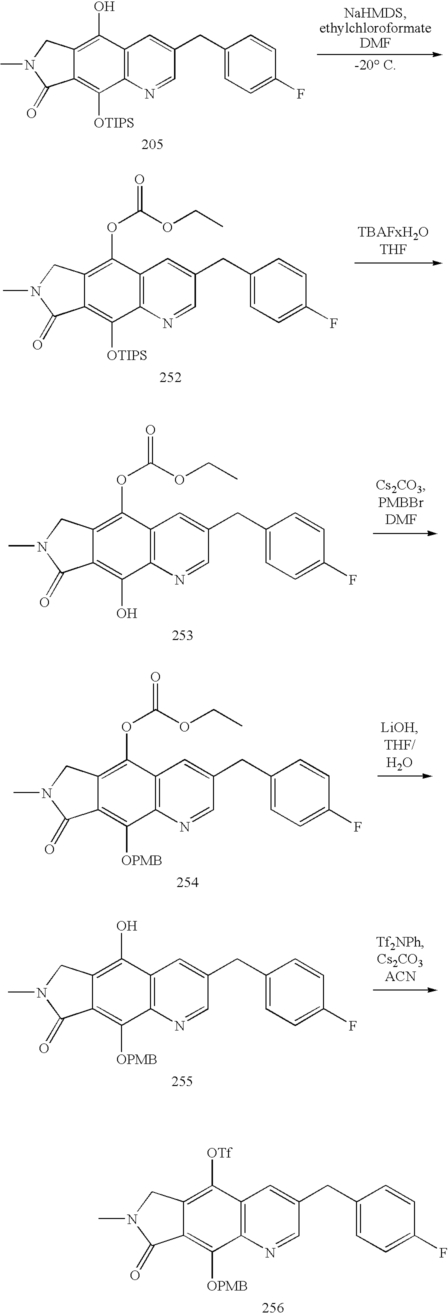

A solution of phenol 205 (7.43 g, 15.03 mmol) in DMF (150 mL, 0.1M) was cooled to approximately −20° C. then treated with NaHMDS (22.55 mL, 1M THF solution). Ethyl chloroformate (1.58 mL, 16.5 mmol) was added dropwise but also very quickly and the reaction was stirred at −20° C. for 10 minutes under nitrogen atmosphere. The reaction was quenched with H₂O and diluted with ethyl acetate. The organic layer was washed with H₂O, sat. NH₄Cl, aqueous LiCl, and brine, then dried (over Na₂SO₄), filtered and concentrated in vacuo to afford the product 252 (8.5 g, quant) with no further purification: 300 MHz ¹H NMR (CDCl₃) δ (ppm): 8.69 (s, 1H), 7.85 (s, 1H), 7.19 (dd, 2H), 7.04 (dd, 2H), 4.37 (s, 2H), 4.36 (q, 2H), 4.175 (s, 2H), 3.175 (s, 3H), 1.52 (sep, 3H), 1.408 (t, 3H), 1.12 (d, 18H); MS: 567 (M+1).

To a solution of intermediate 252 (9.45 g, 16.69 mmol) in THF (167 mL, 0.1M) was added tetrabutylammonium fluoride hydrate (6.55 g, 25.03 mmol). The reaction mixture was stirred under nitrogen atmosphere at room temperature for 0.5 hours upon which it was diluted with ethyl acetate, and quenched with H₂O. The aqueous layer was acidified with 1N HCl (15 mL) and reextracted with ethyl acetate. The combined organic layer was washed with H₂O (2×) and brine, then dried (over Na₂SO₄), filtered and concentrated in vacuo. The crude residue was triturated with hexane/diethyl ether (1/1) to afford clean solid phenol 253 (6.0 g, 88%): 300 MHz ¹H NMR (CDCl₃) δ (ppm): 8.81 (s, 1H), 7.96 (s, 1H), 7.19 (dd, 2H), 7.02 (dd, 2H), 4.48 (s, 2H), 4.36 (q, 2H), 4.194 (s, 2H), 3.199 (s, 3H), 1.418 (t, 3H); MS: 411 (M+1).

The phenol 253 (5.98 g, 14.58 mmol) was dissolved in DMF (146 mL, 0.1M) and treated with Cs₂CO₃ (11.84 g, 36.45 mmol) and stirred for 5 minutes before para-methoxybenzyl bromide (4.18 mL, 29.16 mmol) was added. The reaction was stirred under nitrogen atmosphere at room temperature for 2 hours, upon which the reaction was quenched with water and diluted with ethyl acetate. The organic layer was washed with sat NH₄Cl, aqueous LiCl, and brine, then dried (over Na₂SO₄), filtered and concentrated in vacuo. The crude residue was purified by chromatography on silica gel (3/7—hexane/ethyl acetate) in order to obtain desired product 254 (4.54 mg, 59%): 300 MHz ¹H NMR (CDCl₃) δ (ppm): 8.9 (s, 1H), 7.93 (s, 1H), 7.64 (d, 2H), 7.19 (m, 2H), 7.03 (m, 2H), 6.86 (d, 2H), 5.66 (s, 2H), 4.35 (s, 2H), 4.36 (q, 2H), 4.191 (s, 3H), 3.79 (s, 3H), 3.217 (s, 3H), 1.421 (t, 3H); MS: 531 (M+1).

To a solution of carbonate 254 (4.54 g, 8.56 mmol) dissolved in THF (85.6 mL, 0.1M) was added DMAP (0.523 g, 4.28 mmol) and a solution of LiOH*H2O (1.08 g, 25.7 mmol) in water (43 mL). The reaction was stirred at room temperature for 45 minutes upon which diluted with ethyl acetate and water. The mixture was acidified with 1N HCl (50 mL) and the product was extracted with ethyl acetate twice. The organic layer was washed with water (2×) and brine then dried (over Na₂SO₄), filtered and concentrated in vacuo to give clean product 255 (4.25 g, 100%) with no further purification: 300 MHz ¹H NMR (CDCl₃) δ (ppm): 8.65 (s, 1H), 8.38 (s, 1H), 7.42 (dd, 2H), 7.13 (dd, 2H), 6.95 (dd, 2H), 6.66 (d, 2H), 5.31 (s, 2H), 4.54 (s, 2H), 4.07 (s, 2H), 3.7 (s, 3H), 3.14 (s, 3H); MS: 459 (M+1).

The phenol 255 (4.25 g, 8.56 mmol) was dissolved in acetonitrile (130 mL) then cooled in an ice-bath. To this solution was added Cs₂CO₃ (4.19 g, 12.8 mmol) and the reaction was stirred for 5 minutes upon which N-phenyltrifluomethansulfonimide (3.67 g, 10.3 mmol) was added. The reaction was stirred under nitrogen atmosphere for 3 hours while warning to room temperature. Upon completion, the mixture was diluted with ethyl acetate and quenched with H₂O. The organic layer was washed with sat NH₄Cl, H₂O and brine, then dried (over Na₂SO₄), filtered and concentrated in vacuo. The crude residue was purified by chromatography on silica gel (2/3—hexane/ethyl acetate) to afford the desired triflate 256 (4.265 g, 84%): 300 MHz ¹H NMR (CDCl₃) δ (ppm): 8.96 (s, 1H), 8.02 (s, 1H), 7.6 (d, 2H), 7.20 (dd, 2H) 7.06 (dd, 2H), 6.86 (dd, 2H), 5.75 (s, 2H), 4.59 (s, 2H), 4.22 (s, 2H), 3.79 (s, 3H), 3.24 (s, 3H); 300 MHz ¹⁹F NMR (CDCl₃) δ (ppm) −73.73, −116.225; MS: 591 (M+1).

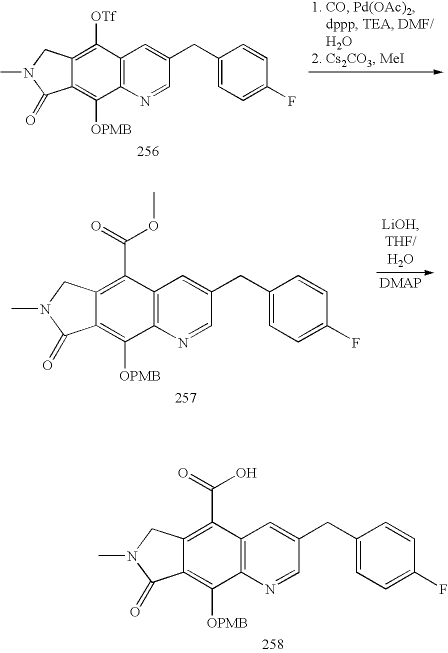

To a solution of triflate 256 (2.0 g, 3.39 mmol) and 1,3-bis(diphenyl-phosphino)propane (DPPP) (670 mg, 1.69 mmol) in DMF (56 mL) and water (5.6 mL) was added Pd(OAc)₂ (230 mg, 1.02 mmol). The solution was degassed under high vacuum (5 minutes) and flushed with carbon monoxide from a balloon. The flushing was repeated several times. TEA (1.13 mL, 8.14 mmol) was introduced. The mixture was heated at 65° C. under CO atmosphere for 2 hours then cooled down to the room temperature. Cs₂CO₃ (2.2 g, 6.78 mmol) and iodomethane (0.844 mL, 13.56 mmol) were added and the reaction mixture was stirred overnight at room temperature under nitrogen atmosphere. The mixture was diluted with ethyl acetate, washed with water, sat NH₄Cl, aq LiCl and brine, then dried (over Na₂SO₄), filtered and concentrated in vacuo. The crude product was purified by chromatography on silica gel column (4/1—hexane/ethyl acetate) to afford the methyl ester product 257 (1.29 g, 77%): 300 MHz ¹H NMR (CDCl₃) δ (ppm): 9.08 (s, 1H), 8.8 (s, 1H), 7.58 (d, 2H), 7.2 (dd, 2H) 7.03 (dd, 2H), 6.82 (dd, 2H), 5.83 (s, 2H), 4.71 (s, 2H), 4.20 (s, 2H), 3.99 (s, 3H), 3.77 (s, 3H), 3.238 (s, 3H); MS: 501 (M+1).

To a solution of ester 257 (1.29 g, 2.58 mmol) dissolved in THF (25.8 mL, 0.1M) was added DMAP (95 mg, 0.774 mmol) and a solution of LiOH.H₂O (325 mg, 7.74 mmol) in water (12.9 mL). The reaction was stirred at room temperature for 4 hours upon which diluted with ethyl acetate and water. The mixture was acidified with 1N HCl (10 mL) and the product was extracted with ethyl acetate twice. The organic layer was washed with brine (2×) then dried (over Na₂SO₄), filtered and concentrated in vacuo to give clean product 258 (1.24 g, 100%) with no further purification: 300 MHz ¹H NMR (CD₃OD) δ (ppm): 9.23 (s, 1H), 8.82 (s, 1H), 7.45 (d, 2H), 7.30 (dd, 2H) 7.06 (dd, 2H), 6.78 (dd, 2H), 5.69 (s, 2H), 4.805 (s, 2H), 4.23 (s, 2H), 3.73 (s, 3H), 3.21 (s, 3H); MS: 487 (M+1).

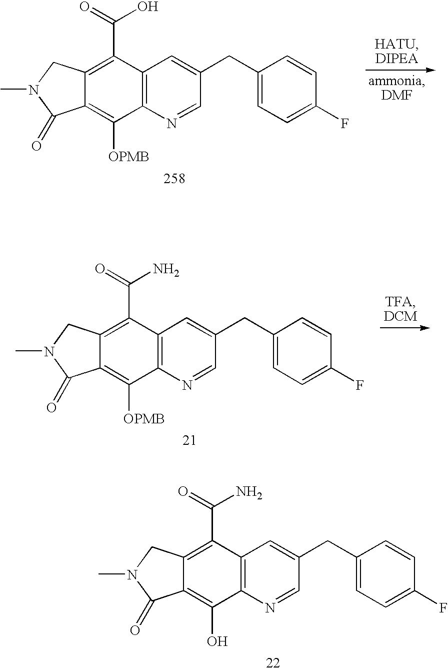

Compound 21: The compound was prepared from the acid 258 using an ammonia solution in Dioxane. This reaction was stirred overnight at room temperature to afford the desired product 21 (42 mg, crude/no purification; from 30 mg of 18): 300 MHz ¹H NMR (CDCl₃) δ (ppm) 8.52 (s, 1H), 8.16 (s, 1H), 7.56 (dd, 2H), 7.20 (m, 2H), 7.04 (m, 2H), 6.9 (d, 2H), 5.31 (s, 2H), 4.29 (s, 2H), 4.04 (s, 2H), 3.84 (s, 3H), 2.68 (s, 3H); MS: 486 (M+1).

Compound 22: The compound was prepared from compound 21 (triturated with ether then purified by reversed phase HPLC; (24 mg, 81% from 30 mg of 18) as the TFA salt: 300 MHz ¹H NMR (DMSO-d₆) δ (ppm) 8.83 (s, 1H), 8.35 (s, 1H), 7.91 (s, 1H), 7.76 (s, 1H), 7.34 (dd, 2H), 7.14 (dd, 2H), 4.60 (s, 2H), 4.22 (s, 2H), 3.04 (s, 3H); 300 MHz ¹⁹F NMR (DMSO-d₆) δ (ppm) −75.03, −117.18; MS: 366 (M+1).

### Example 15

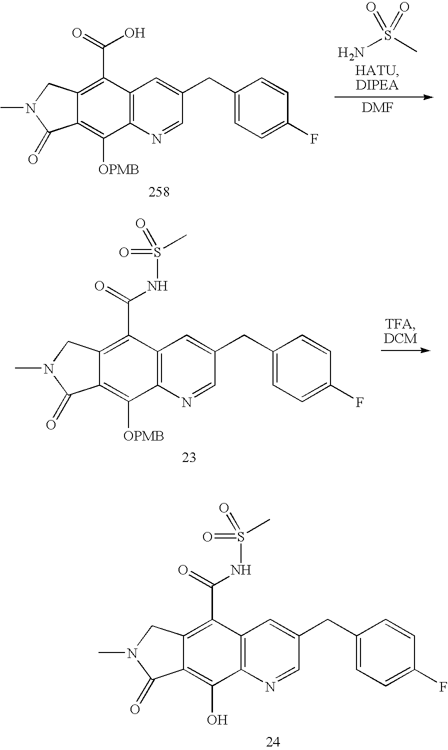

Compound 23: The compound was prepared as illustrated above. The reaction was stirred overnight at room temperature to afford the desired product 23: MS: 564 (M+1).

Compound 24: The compound was made in a similar fashion as described in Example 5 (triturated with ether then purified by reversed phase HPLC to afford the desired product 24 (5.6 mg, 15% from 35 mg of 18) as the TFA salt: 300 MHz ¹H NMR (DMSO-d₆) δ (ppm) 8.87 (s, 1H), 8.28 (s, 1H), 7.35 (dd, 2H), 7.15 (dd, 2H), 5.75 (s, 1H), 4.64 (s, 2H), 4.25 (s, 2H), 3.45 (s, 3H), 3.05 (s, 3H); 300 MHz ¹⁹F NMR (DMSO-d₆) δ (ppm) −74.46, −117.10; MS: 444 (M+1).

### Example 16

### Preparation of Compound 3002

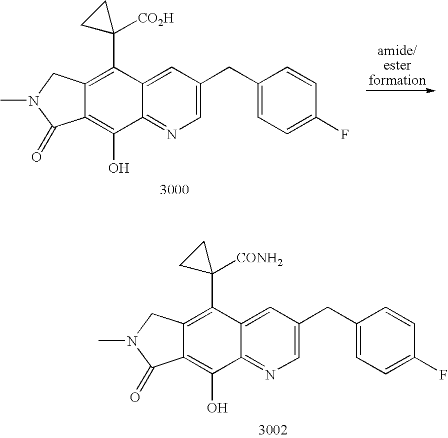

To 120 mg of the carboxylate 3000 in 5 mL anhydrous DCE at 0° C. was added 2 equiv oxalyl chloride and 1 drop of DMF. After 15 minutes, the reaction was concentrated to a residue by use of a rotary evaporator and then high-vacuum. The residue was redissolved in 2 mL DCM and added to 4 mL of a briskly stirred bi-phasic mixture of DCM and conc. Aq. NH₄OH. After 15 additional minutes, the reaction was quenched by portioning between ethyl acetate and 5 aq. citric acid. Solution, followed by washing of the organic layer with brine and drying over sodium sulfate. Concentration gave 110 mg crude product, which upon HPLC purification afforded 21 mg of the product carboxamide 3002: ¹H NMR (300 MHz, d₆-DMSO) shows diagnostic peaks at δ (ppm): 8.78 (s, 1H), 8.31 (s, 1H), 7.26 (m, 2H), 7.11 (m, 2H), 6.80 (s, 1H), 6.42 (s, 1H), 4.64 (d, 1H), 4.35 (d, 1H), 4.21 (s, 2H), 3.05 (s, 3H), 1.85 (m, 1H), 1.32 (m, 1H), 1.08 (m, 1H), 0.79 (m, 1H). MS=406 (M+H).

### Example 17

### Preparation of Compound 3003

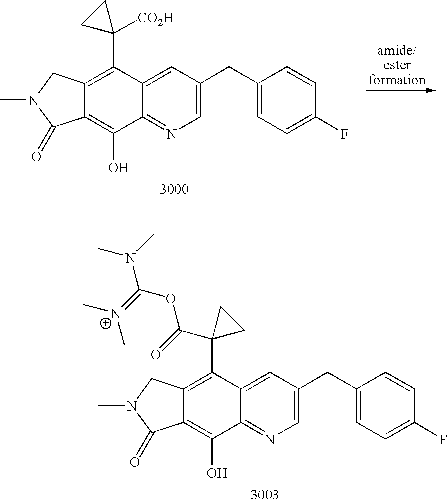

The cyclopropyl carboxylate 3000, 60 mg, was treated with 2 equivalents (120 mg) of HATU in 2 mL DMSO. After stirring for 1 h, the reaction mixture was partitioned between EtOAc and water, and the organic layer dried, concentrated to 110 mg of a crude residue, and purified by HPLC to give 4 mg of the activated ester 3003: ¹H NMR (300 MHz, CD₃OD) shows diagnostic peaks at δ (ppm): 8.92 (s, 1H), 8.57 (s, 1H), 7.36 (m, 2H), 7.08 (m, 2H), 4.60 (d, 1H), 4.31 (s, 2H), 3.35 (s, 3H), 3.15 (s, 12H), 1.99 (m, 1H), 1.85 (m, 1H), 1.52 (m, 1H), 1.26 (m, 1H). MS=505 (M+).

### Example 18

### Preparation of Compound 3004

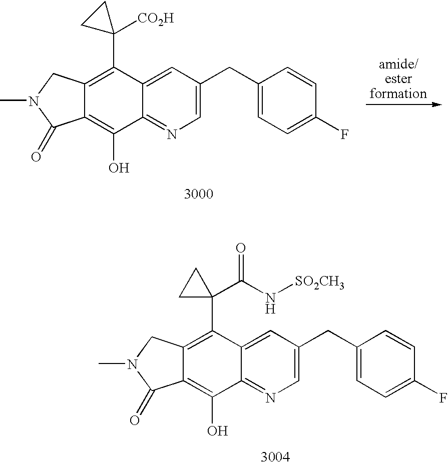

The cyclopropyl carboxylate 3000, 30 mg, was dissolved in 4 mL THF and treated with excess CDI (100 mg). After 20 minutes, 200 μL DBU and 100 mg methyl sulfonamide were added, and the reaction heated to 100° C. (via microwave) for 20 minutes. LC/MS analysis showed complete conversion to the desired product at that time. Concentration of the reaction mixture to a residue via rotary evaporation and purification of the crude material via HPLC furnished 6 mg acyl sulfonamide 3004: ¹H NMR (300 MHz, d₆-DMSO) shows diagnostic peaks at δ (ppm): 10.75 (1H), 8.82 (s, 1H), 8.27 (s, 1H), 7.37 (m, 2H), 7.15 (m, 2H), 4.71 (d, 1H), 4.60 (d, 1H), 4.26 (s, 2H), 3.12 (s, 3H), 3.05 (s, 3H), 1.95 (m, 1H), 1.65 (m, 1H), 1.50 (m, 1H), 1.02 (m, 1H). MS=484 (M+H).

### Example 19

### Preparation of Compound 104

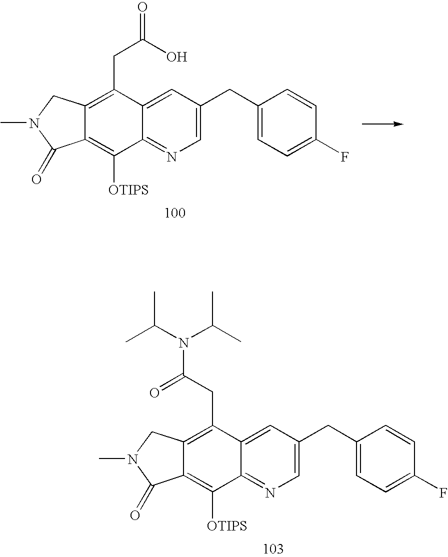

Compound 103 was prepared from compound 100 as illustrated above. 300 MHz ¹H NMR (CDCl₃) δ (ppm): 8.69 (s, 1H), 7.80 (s, 1H), 7.29-7.21 (m, 2H), 7.08-7.00 (m, 2H), 4.37 (s, 2H), 4.16 (s, 2H), 4.02-3.98 (m, 1H), 3.81 (s, 2H), 3.40-3.48 (m, 1H), 3.18 (s, 3H), 2.82 (s, 3H), 1.49-1.52 (m, 3H), 1.10-1.15 (m, 12H). 1.12-1.15 (m, 9H).

¹⁹F NMR (CDCl₃) δ (ppm): −116.87

MS: 620.51 (M+1).

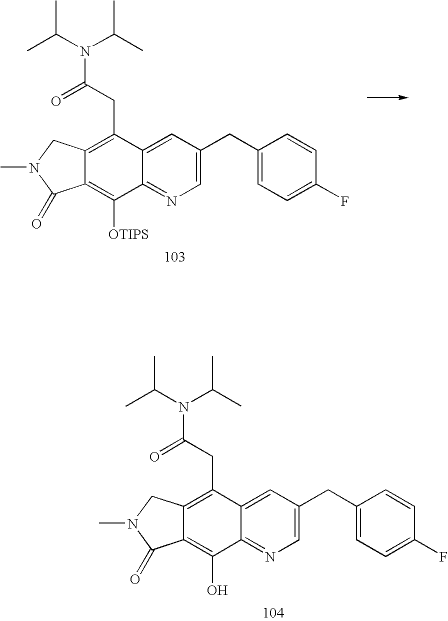

Compound 104 was prepared from compound 103 as illustrated above. 300 MHz ¹H NMR ((DMSO-d₆) δ (ppm): 8.83 (s, 1H), 7.97 (s, 1H), 7.33-7.25 (m, 2H), 7.18-7.08 (m, 2H), 4.39 (s, 2H), 4.20 (s, 2H), 3.92 (s, 2H), 3.60-3.68 (m, 2H), 3.04 (s, 3H), 2.82 (s, 3H), 1.20-1.0 (m, 12H). ¹⁹F NMR ((DMSO-d₆) δ (ppm): −74.99, −117.31

MS: 464.71 (M+1).

### Example 20

### Preparation of Compound 2999

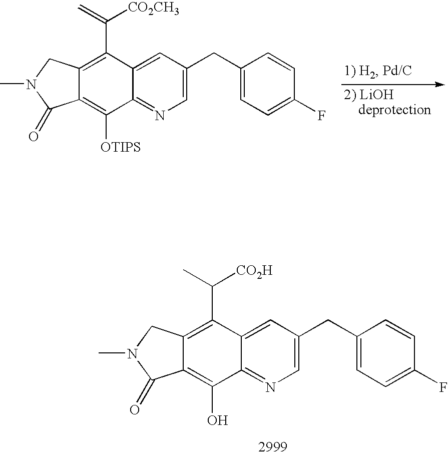

To the acrylate ester, 30 mg, 0.05 mmol, in a 10:1 solution of EtOH:EtOAc, is added 30 mg 10% wt Pd/C. The mixture was subjected to H₂ via balloon for a period of 4 h, at which time LC/MS analysis indicated that hydrogenation was complete. The reaction was filtered and concentrated, and the crude residue, 30 mg, subjected directly to LiOH deprotection at elevated temperature under the standard conditions reported herein. After 90 minutes, the reaction was diluted with 50 mL EtOAc, washed with 5% aq. citric acid, dried over Na₂SO₄ and concentrated. After purification by trituration from DCM/hexanes, 9 mg of the methyl carboxylate 2999 was obtained: ¹H NMR (300 MHz, CD₃OD) shows diagnostic peaks at δ (ppm): 8.76 (s, 1H), 8.35 (s, 1H), 7.30 (m, 2H), 7.03 (m, 2H), 4.61 (dd, 2H), 4.33 (m, 4H), 3.20 (s, 3H), 3.18 (s, 3H), 1.55 (d, 3H). MS=395 (M+H).

### Example 21

### Preparation of Compound 128

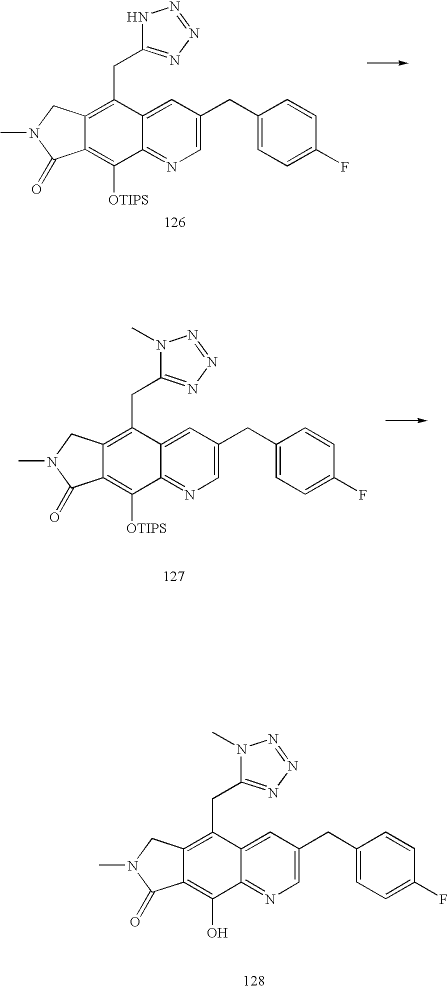

Compound 126 (40 mg, 0.07 mmol, 1 equiv.) was dissolved in benzene/MeOH (3 mL/1 mL) followed by addition of TMSCHN₂ (55 μL, 0.11 mmol, 1.5 equiv.). After the reaction was complete, it was concentrated in vacuo. Crude compound 127 (155 mg, 0.25 mmol, 1 equiv) was stirred in THF (5 mL) and water (2 mL) before adding TFA (0.5 mL). The reaction was warmed to 50° C. After the reaction was complete, it was concentrated in vacuo and azoetroped with toluene. It was purified by HPLC. 400 MHz ¹H NMR ((DMSO-d₆) δ (ppm) 8.75 (s, 1H), 8.42 (s, 1H), 7.31-7.26 (m, 2H), 7.12-7.07 (m, 2H), 4.52 (s, 2H), 4.47 (s, 2H), 4.15 (s, 3H), 4.14 (s, 2H), 2.99 (s, 3H). 300 MHz ¹⁹F NMR ((DMSO-d₆) δ (ppm): −79.76, −118.25. MS: 419.29 (M+1).

### Example 22

### Preparation of Compound 31

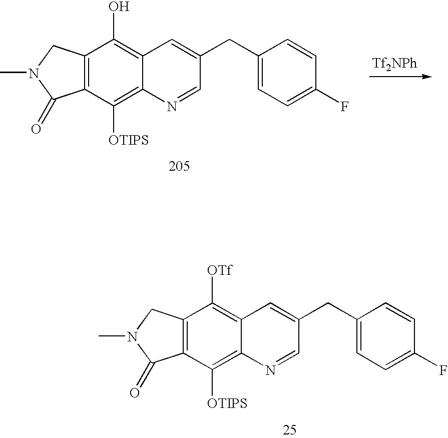

Cesium carbonate (5.2123 g, 15.997 mmol) was added to a 0° C. suspension of 205 (5.15 g, 10.41 mmol) in 100 mL acetonitrile. The reaction was stirred for 19 min. before adding N-phenyltrifluoromethanesulfonimide (4.4561 g, 12.47 mmol). After 1.5 h the ice bath was removed and the reaction was allowed to warm to ambient temperature. Evaluation by LC/MS indicated that the reaction was complete in 4.25 h. The reaction mixture was diluted into 400 mL of ethyl acetate, washed with 500 mL of water which was back extracted with 200 mL of ethyl acetate. The pooled ethyl acetate extracts were washed with water (3×400 mL), 400 mL of saturated NH₄Cl (aq) and 400 mL of brine before drying (Na₂SO₄), filtering and evaporating in vacuo at 30° C. Purification of the crude residue (7 g) was accomplished on silica gel (CombiFlash 330 g, hexane/ethyl acetate) to afford 216, 5.0 g. ¹H NMR (300 MHz, CDCl₃) d (ppm): 8.76 (s, 1H), 7.97 (s, 1H), 7.24 (m, 2H), 7.07 (m, 2H), 4.53 (s, 2H), 4.2 (s, 2H) 3.20 (s, 3H), 1.53 (m, 3H), 1.13 (d, 6H, J=7.6 Hz); LC/MS (m/z) 627.00 [M+H]⁺.

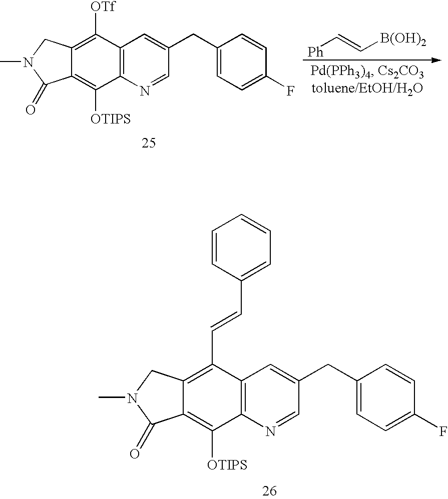

Compound 26: To a solution of triflate 25 (500 mg, mmol) dissolved in a mixture of toluene (10.4 mL) and ethanol (5.2 mL) and water (2.4 mL) was added Cs₂CO₃ (650 mg, 1.99 mmol), Tetrakis(triphenylphosphine)palladium (138 mg, 0.120 mmol) then Trans-2-phenylvinylboronic acid (177 mg, 1.20 mmol). The reaction was sealed in an appropriate veseel and heated in a microwave at 150° C. for 10 minutes, at which the starting material was fully consumed. The process was repeated 4 times to react a total of 2 g of triflate 25. The reactions were combined then diluted with ethyl acetate and quenched with 5% Citric Acid solution. The layers were separated and the aqueous layer was extracted with ethyl acetate. The combined organic layer was washed with water, saturated NaHCO₃ and brine, then dried (over Na₂SO₄), filtered (with celite) and concentrated in vacuo. The crude residue was purified by ISCO flash column chromatography with 3/7 EtOAc/Hexanes to afford the desired product 26 (1.07 g, 57%, +185 mg of impure fractions): 300 MHz ¹H NMR (CDCl₃) δ (ppm) 8.70 (s, 1H), 8.21 (s, 1H), 7.6-7.3 (m, 6 μl), 7.2 (m, 2H), 7.03 (s, 2H), 6.83 (d, 1H), 4.54 (s, 2H), 4.18 (s, 2H), 3.22 (s, 3H), 1.54 (m, 3H), 1.14 (d, 18H); MS: 581 (M+1).

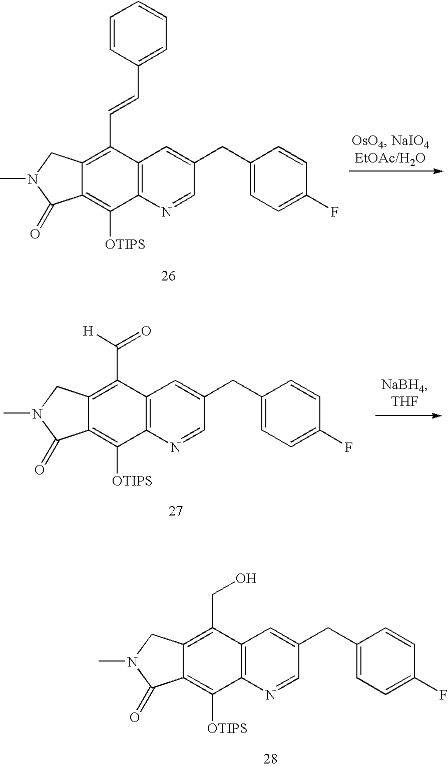

Compound 27: To a solution of styrene 26 (855 mg, 1.47 mmol) dissolved in ethyl acetate (29.5 mL) was added water (14.7 mL) then OsO₄ (0.924 mL, 0.074 mmol, 2.5 wt. % solution in tert-butanol) and lastly NaIO₄ (788 mg, 3.68 mmol). The reaction was stirred at room temperature for 4 hours upon which diluted with ethyl acetate and quenched with water. The layers were separated and the aqueous layer was extracted with ethyl acetate. The combined organic layer was washed with 10% NaHSO₃, and brine (×2), then dried (NaSO₄), filtered (with celite) and concentrated in vacuo to afford the crude aldehyde 27 with no further purification or characterization and taken forward immediately; MS: 507 (M+1).

Compound 28: The crude aldehyde 27 (0.517 mmol) was dissolved in THF (5.2 mL) and cooled in an ice-water bath. To the reaction was added NaBH₄ (110 mg, 2.95 mmol) and then allowed to stir under nitrogen atmosphere for 1.5 hours. At which point, the reaction was diluted with ethyl acetate and quenched with 5% Citric Acid solution. The layers were separated and the aqueous layer was extracted with ethyl acetate. The combined organic layer was washed with water, saturated NaHCO₃ and brine, then dried (over Na₂SO₄), filtered (with celite) and concentrated in vacuo. The crude residue was purified by ISCO flash column chromatography with 6/4 EtOAc/Hexanes to afford the desired product 28 (450 mg, 60%—2 steps): 400 MHz ¹H NMR (CDCl₃) δ (ppm) 8.70 (s, 1H), 8.38 (s, 1H), 7.23 (dd, J=3 Hz, J=5.4 Hz, 2H), 7.03 (dd, J=8.4 Hz, 2H), 4.93 (s, 2H), 4.30 (s, 2H), 4.19 (s, 2H), 2.63 (s, 3H), 1.53 (sep, J=7.5 Hz, 3H), 1.13 (d, J=7.8 Hz, 18H); MS: 509 (M+1).

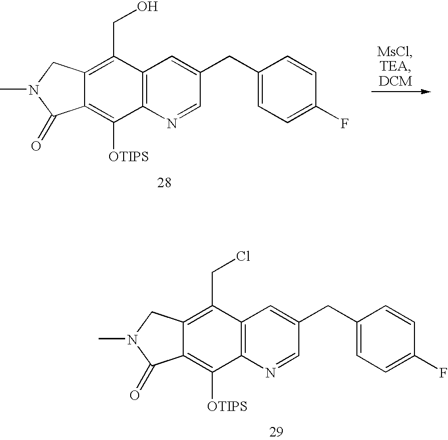

Compound 29: To a solution of benzyl alcohol 28 (450 mg, 0.886 mmol) dissolved in dichloromethane (9 mL) cooled in an ice-water bath was added Triethylamine (1.23 mL, 8.86 mmol) then Methanesulfonyl chloride (0.55 mL, 7.09 mmol) dropwise. The reaction was stirred for 2 hours under a nitrogen atmosphere while warming to room temperature. At which point, the solvent was removed in vacuo then the reaction was diluted with ethyl acetate and quenched with sat. NH₄Cl. The layers were separated and the aqueous layer was extracted with ethyl acetate. The combined organic layer was washed with brine (×2), then dried (over NaSO₄), filtered and concentrated in vacuo. The crude residue was purified by running a short silica plug using 1/3 EtOAc/Hexanes to afford the desired product 29 (392 mg, 84%): 400 MHz ¹H NMR (CDCl₃) δ (ppm) 8.69 (s, 1H), 8.08 (s, 1H), 7.20 (dd, J=3.2 Hz, J=5.2 Hz, 2H), 7.03 (dd J=8.8 z, 2H), 4.85 (s, 2H), 4.48 (s, 2H), 4.19 (s, 2H), 3.19 (s, 3H), 1.50 (sep, J=7.6 Hz, 3H), 1.10 (d, J=7.2 Hz, 18H); MS: 527 (M+1).

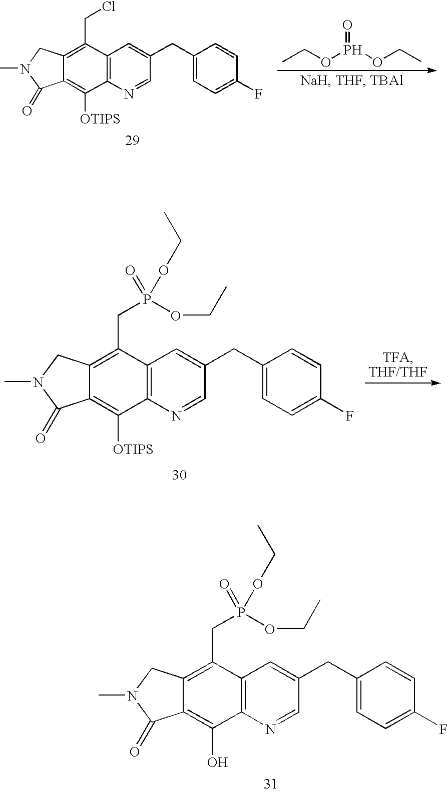

Compound 30: A suspension of Sodium hydride (9 mg, 0.225 mmol) in THF (0.5 mL) was cooled in an ice-water bath. To this suspension was added diethyl phosphate (20 μL, 0.149 mmol) pre-dissolved in 0.2 mL THF. The reaction was stirred for 20 min in the bath under nitrogen atmosphere. At which point, a pre-made solution of benzyl chloride 29 (60 mg, 0.119 mmol, crude) and tetra-butylammonium iodide (45 mg, 0.121 mmol) in THF (1 mL) was added to the reaction and then stirred while warming to room temperature. After 30 minutes very little product had formed. Therefore, a fresh batch of sodium phosphate was made [17 mg of NaH and 60 μL of diethyl phosphate was combined in 0.7 mL of THF], then added to the reaction mixture. After 10 minutes, starting material was consumed. The reaction was then diluted with ethyl acetate and quenched with H₂O. The aqueous layer was extracted with ethyl acetate and the combined organic layer was washed with sat. NH₄Cl and brine, then dried (over Na₂SO₄), filtered and concentrated in vacuo to afford the crude product 30 (70 mg) with no further purification or characterization; MS: 629 (M+1) and significant amount of TIPS loss: 473 (M+1).

Compound 31: Compound 31 was prepared from compound 30 as illustrated above (triturated with ether/hexane then purified by reversed phase HPLC to afford the desired product 31 (10 mg, 15% from 60 mg of 29) as the TFA salt: 300 MHz ¹H NMR (CDCl₃) δ (ppm) 8.85 (s, 1H), 8.16 (s, 1H), 7.21 (dd, J=3.0 Hz, J=5.7 Hz, 2H), 7.02 (dd, J=8.4 Hz, 2H), 4.6 (d, J=3.3 Hz, 2H), 4.21 (s, 2H), 4.0-3.8 (m, 4H), 3.39 (d, J=21 Hz, 3H), 3.20 (s, 3H), 1.12 (t, J=7.2 Hz, 6H); 300 MHz ³¹P NMR (CDCl₃) δ (ppm) 25.30; MS: 473 (M+1).

### Example 23

### Preparation of Compound 36

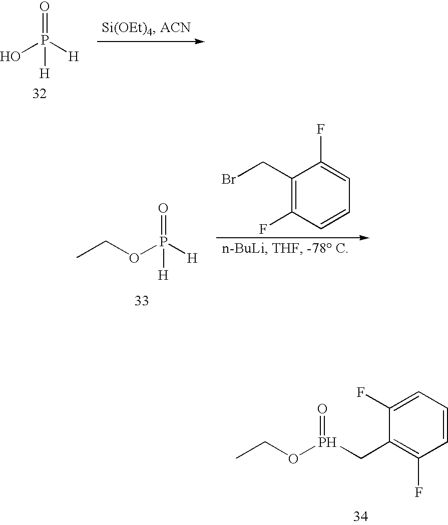

Compound 33: Following literature precedence [J. Organomet. Chem., 643-644 (2002) 154-163], Concentrated hypophosphorus acid 32 [commercially available as 50 wt. % Aq. Solution] (1.64 g, 24.88 mmol, 3.28 g of solution) was dissolved in acetonitrile (45.88 mL, 0.5M). Tetra-butoxysilane (3.89 mL, 17.4 mmol, ***Caution: may cause blindness) was added dropwise while a water bath was used to insulate the reaction. Upon addition, the reaction was heated to reflux (80° C.) for 2 hours under nitrogen atmosphere. At which point, the reaction was complete, as monitored by ³¹P NMR, and then was cooled to room temperature to afford the desired intermediate 33 (stored as a 0.5M acetonitrile solution); 300 MHz ³¹P NMR (CDCl₃) δ (ppm) 14.53.

Compound 34: Adapted from Synthesis, 2006, No. 2 325-331, Intermediate 33 (10 mL, 5.0 mmol, 0.5M acetonitrile solution) and 2,6-Difluorobenzylbromide (690 mg, 3.33 mmol) was dissolved in THF (55 mL) and cooled to −78° C. under nitrogen atmosphere. n-Butyllithium (4.13 mL, 6.6 mmol, 1.6M Hexane solution) was added dropwise while the internal temperature was carefully monitored. Upon addition, the reaction stirred in the −78° C. bath, as it warned, for 2 hours then stirred for 30 min in an ice-bath. The reaction was stored in a −10° C. freezer overnight then diluted with ethyl acetate and quenched with 20% NaHSO₄. The aqueous layer was extracted with ethyl acetate and the combined organic layer was washed with brine, then dried (over Na₂SO₄), filtered and concentrated in vacuo. The crude residue was purified by ISCO flash column chromatography with 4/6 EtOAc/Hexanes to afford the desired product 34 (330 mg, 45% based on Limiting Reagent): 300 MHz ¹H NMR (CDCl₃) δ (ppm) 8.08 and 6.22 (d, 1H), 7.27 (m, 1H), 6.94 (m, 2H), 4.14 (q, 2H), 4.21 (s, 2H), 3.30 (d, J=18.3 Hz, 4H), 1.34 (t, 3H); 300 MHz ³¹P NMR (CDCl₃) δ (ppm) 31.44.

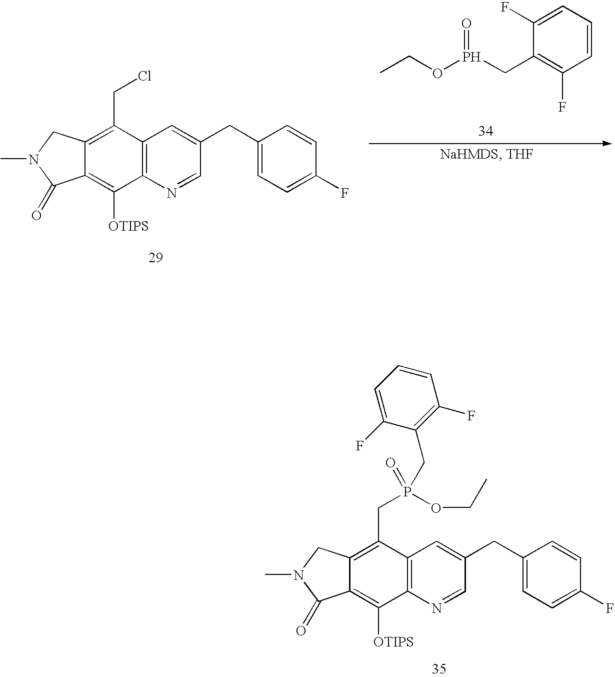

Compound 35: To a solution of phosphonous ester 34 (121 mg, 0.55) dissolved in THF (2.0 mL) and cooled in an ice-water bath was added Sodium bis(trimethylsilyl)amide (0.55 mL, 0.55 mmol, 1M THF solution). The reaction was pre-stirred in the bath for 2-3 minutes before a solution of benzyl chloride 29 (90 mg, crude, 0.157 mmol) in THF (3.5 mL) was added dropwise. The reaction was stirred under nitrogen atmosphere in the ice-water bath for 15 minutes, upon which it was diluted with ethyl acetate and quenched with sat. NH₄Cl. The aqueous layer was extracted with ethyl acetate and the combined organic layer was washed with brine (×2), then dried (over Na₂SO₄), filtered and concentrated in vacuo. The crude residue was purified by ISCO flash column chromatography with 7/3 EtOAc/Hexanes to afford the desired product 35 (60 mg, 54%—2 steps from benzyl alcohol 28): 300 MHz ¹H NMR (CDCl₃) δ (ppm) 8.66 (s, 1H), 7.84 (s, 1H), 7.4-7.1 (m, 4H), 7.01 (m, 3H), 4.42 (m, 2H), 4.09 (s, 2H), 3.5 (m, 2H), 3.37 (m, 2H), 3.16 (s, 3H), 1.50 (sep, J=6.9 Hz, 3H), 1.10 (d, J=4.8 Hz, 18H); 300 MHz ³¹P NMR (CDCl₃) δ (ppm) 45.46; MS: 711 (M+1).

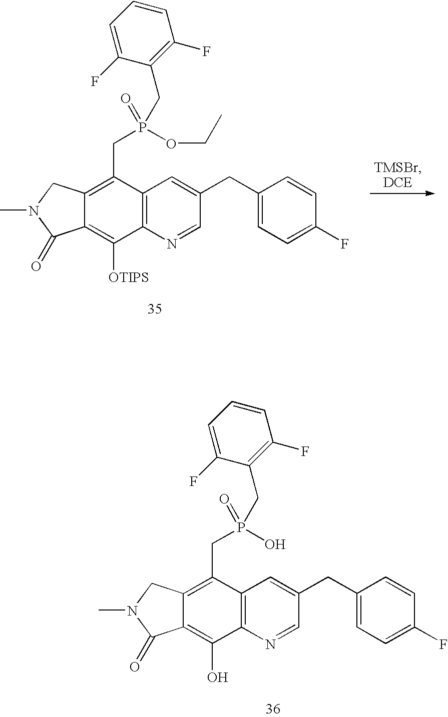

Compound 36: To a solution of phosphinate 35 (33.5 mg, 0.047 mmol) dissolved in dichloroethane (0.5 mL) was treated with trimethylsilyl bromide (61 μL, 0.47 mmol). The reaction was heated to 60° C. and stirred for 2 hours under nitrogen atmosphere. At which point the starting material was consumed and the reaction was quenched with methanol (1 mL). The volatiles were removed in vacuo and the crude residue was purified by reversed phase HPLC (Phenomenex Gemini Axia-packed column with 0.1% TFA in the mobile phase) to afford the desired product 36 (15 mg, 50%) as a TFA salt: 400 MHz ¹H NMR (DMSO-d₆) δ (ppm) 8.74 (s, 1H), 8.40 (s, 1H), 7.31 (m, 3H), 7.06 (m, 4H), 4.50 (s, 2H), 4.14 (s, 2H), 3.50 (d, J=14.4 Hz, 2H), 3.16 (d, J=15.6 Hz, 2H), 3.01 (s, 3H); 400 MHz ¹⁹F NMR (DMSO-d₆) δ (ppm) −74.50, −112.98, −117.37; 300 MHz ³¹P NMR (DMSO-d₆) δ (ppm) 39.53; MS: 527 (M+1).

### Example 24

### Preparation of Compound 37

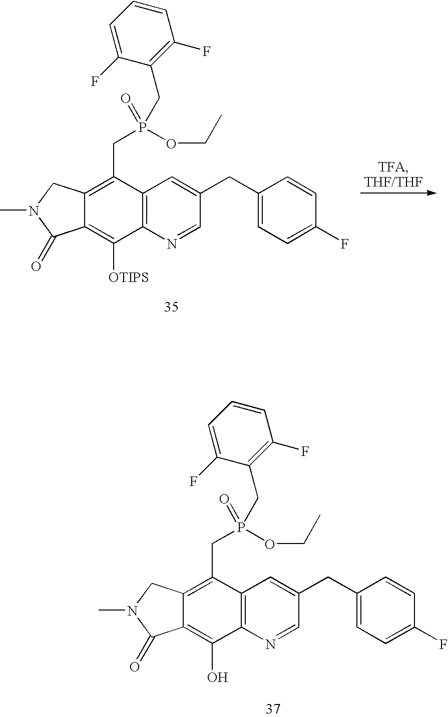

Compound 37: The compound was made in a similar fashion as compound 10 then purified by reversed phase HPLC (Phenomenex Gemini Axia-packed column with 0.1% TFA in the mobile phase) to afford the desired product 37 (10 mg, 56% from 19 mg of 35) as the TFA salt: 300 MHz ¹H NMR (CDCl₃) δ (ppm) 9.06 (s, 1H), 8.12 (s, 1H), 7.37 (m, 1H), 7.15 (dd, J=5.4 Hz, 2H), 7.04 (m, 4H), 4.55 (AB, J=58.8 Hz, J=17.4 Hz, 2H), 4.17 (s, 2H), 3.76 (m, 1H), 3.60 (m, 1H), 3.4 (m, 4H), 0.79 (t, J=7.2 Hz, 3H); 300 MHz ¹⁹F NMR (CDCl₃) δ (ppm) −76.41, −113.23, −115.77; 300 MHz ³¹P NMR (CDCl₃) δ (ppm) −47.47; MS: 555 (M+1).

### Example 25

### Preparation of Compound 14

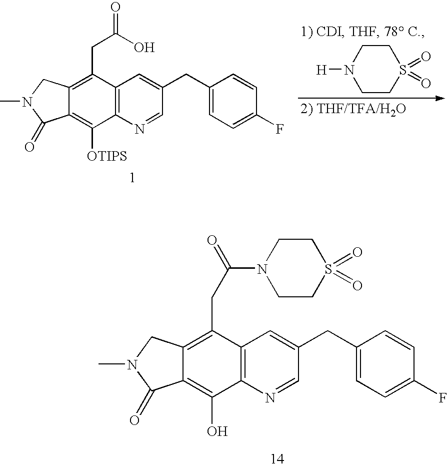

Compound 14 was synthesized from 1 and purified by flash chromatography before subjecting to TFA promoted TIPS removal. Compound 14: 5-[2-(1,1-Dioxo-116-thiomorpholin-4-yl)-2-oxo-ethyl]-3-(4-fluoro-benzyl)-9-hydroxy-7-methyl-6,7-dihydro-pyrrolo[3,4-g]quinolin-8-one. ¹H-NMR (400 MHZ; DMSO-d₆): d 8.76 (s, 1H), 8.22 (s, 1H), 7.31 (t, 2H), 7.10 (t, 2H) 4.41 (s, 2H), 4.19-4.08 (m, 6H), 3.83 (s, 2H), 3.37 (s, 2H), 3.10 (s, 2H), 3.03 (s, 3H). ¹⁹F-NMR (400 MHZ; DMSO-d₆): −117.10, −75.27 MS [M+H]+=498.42

### Example 26

### Preparation of Compound 19

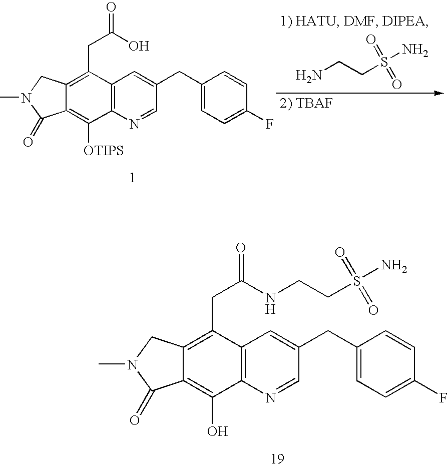

Compound 19 was synthesized from 1 and purified by flash chromatography before subjecting to TBAF promoted TIPS removal. 2-[3-(4-Fluoro-benzyl)-9-hydroxy-7-methyl-8-oxo-7,8-dihydro-6H-pyrrolo[3,4-g]quinolin-5-yl]-N-(2-sulfamoyl-ethyl)-acetamide. ¹H-NMR (300 MHZ; DMSO-d₆): δ 8.79 (s, 1H), 8.39 (s, 1H), 8.16 (br, 1H), 7.95 (s, 1H), 7.84 (br, 1H), 7.37 (t, 2H), 7.17 (m, 3H), 6.88 (s, 1H), 4.52 (s, 2H), 4.20 (s, 2H), 3.79 (s, 2H), 3.05 (s, 3H), 2.88-2.68 (m, 4H). ¹⁹F-NMR (300 MHZ; DMSO-d₆): −148.794, −74.33, −71.92, −69.39. MS [M+H]+=487.45

### Example 27

### Preparation of Compound 115

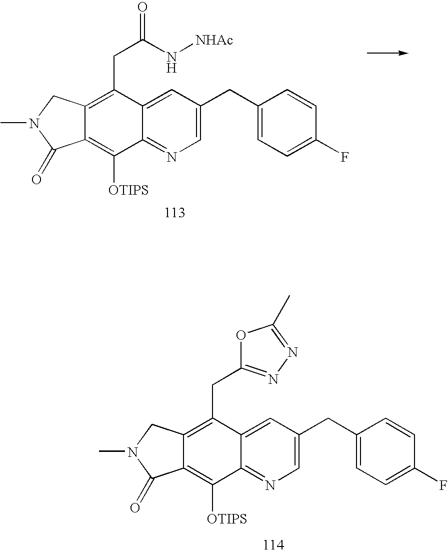

Bull. Korean. Chem. Soc., 2001, 22, 1153.

Into a flask containing acetamide 113 (80 mg, 0.13 mmol, 1 equiv.) was added CH₂Cl₂ (3 mL) followed by PPh₃ (88 mg, 0.34 mmol, 2.5 eqiuv.), CCl₄ (104 μL, 1.08 mmol, 8 equiv) and lastly TEA (56 μL, 0.41 mmol, 3 equiv.). After the reaction was complete, it was diluted with ethyl acetate and quenched with water. The organic layer saturated was washed with NH₄Cl, aqueous LiCl, and brine, then dried (Na₂SO₄), filtered and concentrated. The residue was purified by chromatography on silica gel (Hexanes/Ethyl acetate (7/3)) to afford the desired product 114. 300 MHz ¹H NMR (CDCl₃) δ (ppm): 8.70 (s, 1H), 8.19 (s, 1H), 7.29-7.21 (m, 2H), 7.08-7.00 (m, 2H), 4.53 (s, 2H), 4.37 (s, 2H), 4.19 (s, 2H), 3.22 (s, 3H), 2.41 (s, 3H), 1.49-1.52 (m, 3H), 1.12-1.15 (m, 9H). ¹⁹F NMR (CDCl₃) δ (ppm): −116.64 MS: 575.37 (M+1).

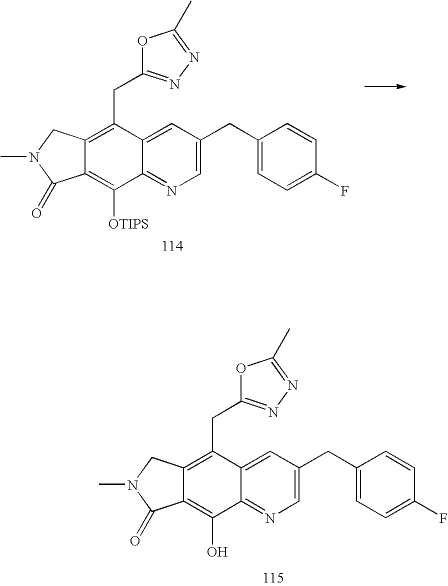

Compound 115 was made in a manner similar to an example shown above. 300 MHz ¹H NMR ((DMSO-d₆) δ (ppm): 8.84 (s, 1H), 8.51 (s, 1H), 7.33-7.25 (m, 2H), 7.15-7.05 (m, 2H), 4.56 (s, 2H), 4.55 (s, 2H), 4.21 (s, 2H), 3.06 (s, 3H), 2.36 (s, 3H).

¹⁹F NMR ((DMSO-d₆) δ (ppm): −75.3, −117.27

MS: 419.37 (M+1)

### Example 28

### Preparation of Compound 117

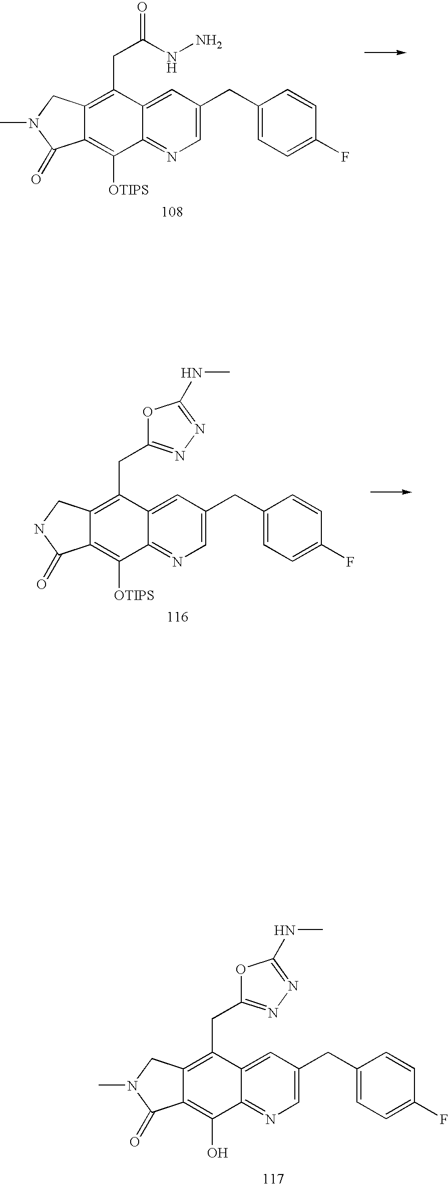

Compound 108 (58 mg, 0.11 mmol, 1 equiv.) was dissolved in THF (4 mL, 0.025 M) and followed by addition of methyl isothiocyanate (38 mg, 0.52 mmol, 5 equiv.) before being warmed to 60° C. After the reaction was complete, it was diluted with ethyl acetate and quenched with water. The organic layer saturated was washed with NH₄Cl, aqueous LiCl, and brine, then dried (Na₂SO₄), filtered and concentrated down. Crude 116 was then dissolved in THF (2 mL, 0.05 M) followed by addition of pyridine (26 μL, 0.32 mmol, 6 equiv.) and TsCl (31 mg, 0.16 mmol, 3 equiv.). The reaction was stirred at 70° C. After the reaction was complete, it was diluted with ethyl acetate and quenched with water. The organic layer saturated was washed with NH₄Cl, aqueous LiCl, and brine, then dried (Na₂SO₄), filtered and concentrated down. 117 was obtained after HPLC purification. 400 MHz H NMR ((DMSO-d₆) δ (ppm): 8.82 (d, J=1.5 Hz, 1H), 8.52 (s, 1H), 7.39-7.34 (m, 2H), 7.15-7.05 (m, 2H), 6.90-6.75 (m, 1H), 4.54 (s, 2H), 4.41 (s, 2H), 4.21 (s, 2H), 3.05 (s, 3H), 2.68 (d, J=4.2 Hz, 3H). ¹⁹F NMR ((DMSO-d₆) δ (ppm): −75.33, −117.26.

MS: 434.40 (M+1)

### Example 29

### Preparation of Compound 109

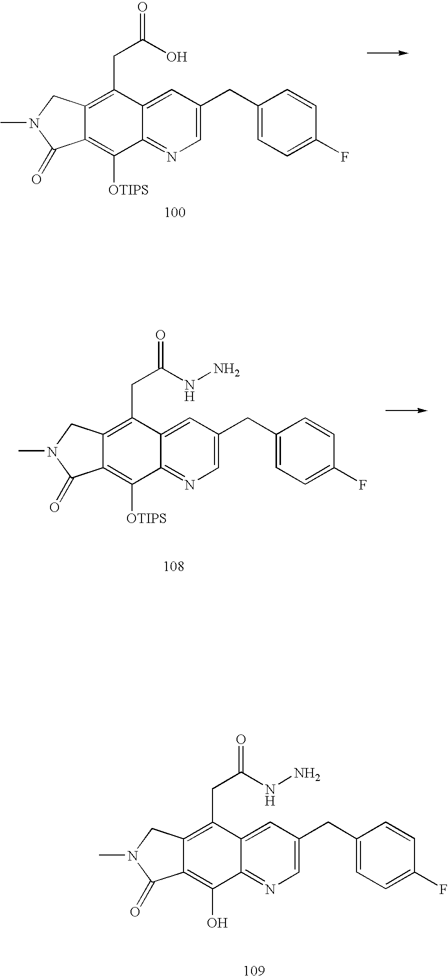

Compound 109 was made in a manner similar to the example shown above. Compound 108 was not purified or fully characterized but was carried on to the next reaction. 400 MHz ¹H NMR (CDCl₃) δ (ppm): 10.78 (bs, 1H), 8.77 (s, 1H), 8.44 (s, 1H), 7.37-7.28 (m, 2H), 7.25-7.18 (m, 2H), 4.50 (s, 2H), 4.32 (s, 2H), 4.07 (m, 1H), 3.81 (s, 2H), 3.03 (s, 3H). ¹⁹F NMR (CDCl₃) δ (ppm): −85.79, −116.87 (may not have been tuned/calibrated)

MS: 395.38 (M+1).

### Example 30

### Preparation of Compound 119

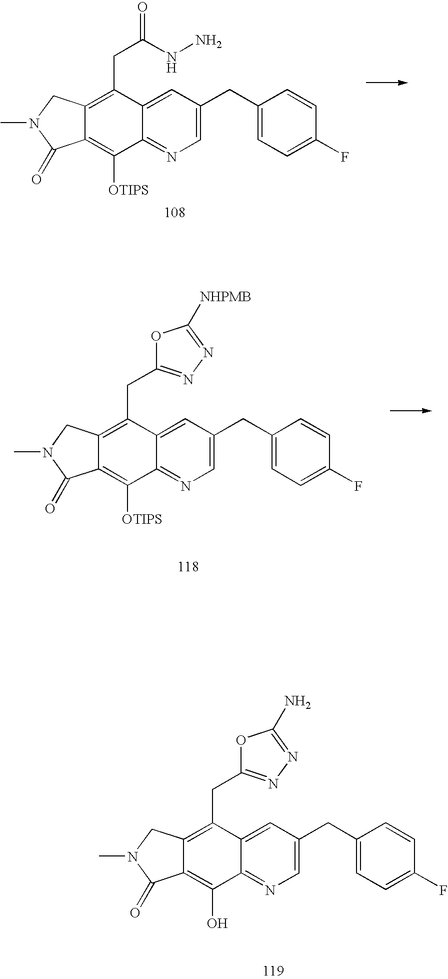

Compound 108 (250 mg, 0.45 mmol, 1 equiv.) was dissolved in THF (5 mL, 0.1 M) and followed by addition of p-methoxybenzyl isothiocyanate (155 μL, 0.99 mmol, 2.2 equiv.) and warmed to 60° C. After the reaction was complete, it was diluted with ethyl acetate and quenched with water. The organic layer saturated was washed with NH₄Cl, aqueous LiCl, and brine, then dried (Na₂SO₄), filtered and concentrated down. Crude 118 was then dissolved in THF (5 mL, 0.1 M) followed by addition of pyridine (110 μL, 1.36 mmol, 3 equiv.) and TsCl (130 mg, 0.68 mmol, 1.5 equiv.). The reaction was stirred at 70° C. After the reaction was complete, it was diluted with ethyl acetate and quenched with water. The organic layer saturated was washed with NH₄Cl, aqueous LiCl, and brine, then dried (Na₂SO₄), filtered and concentrated down. 119 was obtained after HPLC purification. 400 MHz ¹H NMR ((DMSO-d₆) δ (ppm): 8.82 (d, J=1.6 Hz, 1H), 8.47 (s, J=1.6 Hz, 1H), 7.39-7.34 (m, 2H), 7.05 (bs, 2H), 7.15-7.05 (m, 2H), 6.87 (d, J=7.2 Hz, 1H), 4.51 (s, 2H), 4.38 (s, 2H), 4.18 (s, 2H), 3.03 (s, 3H). ¹⁹F NMR ((DMSO-d₆) δ (ppm): −86.33, −120.03.

MS: 420.38 (M+1).

### Example 31

### Preparation of Compound 15

### Imidazole 5001

Use of imidazole as nucleophile gave 8 mg 5001: ¹H NMR (400 MHz, CDCl₃) shows diagnostic peaks at δ (ppm): 8.82 (s, 1H), 8.36 (s, 1H), 7.94 (s, 1H), 7.20 (m, 2H), 7.05 (m, 2H), 5.63 (s, 2H), 4.55 (s, 2H) 4.18 (s, 2H), 3.15 (s, 3H) MS=403.1 (M+H).

### Example 33

### Preparation of Compound 5002

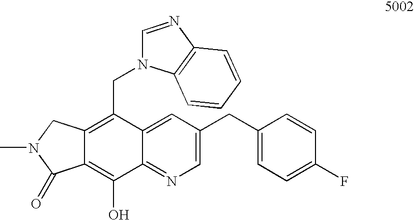

Compound 5002 was prepared following the procedure outlined in Example 32. Use of benzimidazole as nucleophile gave 12 mg 5002: ¹H NMR (400 MHz, d3-acetonitrile) shows diagnostic peaks at δ (ppm): 8.85 (s, 1H), 8.24 (s, 1H), 7.85 (m, 1H), 7.74 (m, 1H), 7.48 (m, 1H), 7.12 (m, 2H), 6.97 (m, 2H), 5.84 (s, 1H), 4.56 (s, 2H) 4.08 (s, 2H), 3.05 (s, 3H).

MS=453 (M+H).

### Example 34

### Preparation of Compound 5003

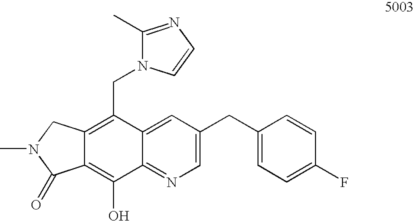

Compound 5003 was prepared following the procedure outlined in Example 32

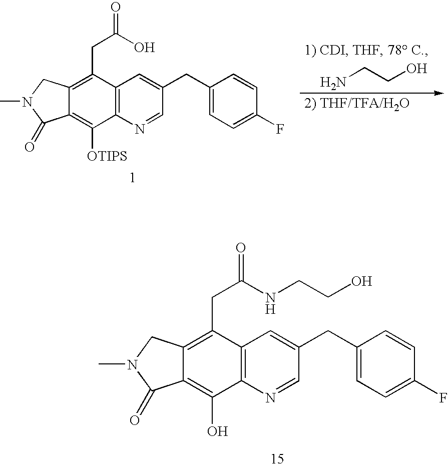

Compound 15 was synthesized from 1 and purified by flash chromatography before subjecting to TFA promoted TIPS removal. Compound 15: 2-[3-(4-Fluoro-benzyl)-9-hydroxy-7-methyl-8-oxo-7,8-dihydro-6H-pyrrolo[3,4-g]quinolin-5-yl]-N-(2-hydroxy-ethyl)-acetamide. ¹H-NMR (300 MHZ; DMSO-d₆): δ 8.79 (s, 1H), 8.47 (s, 1H), 8.14 (s, 1H), 7.36 (t, 2H), 7.13 (t, 2H), 4.53 (s, 2H), 4.20 (s, 1H), 3.79 (s, 2H), 3.38 (t, 2H), 3.08 (t, 2%), 3.05 (s, 3H). ¹⁹F-NMR (300 MHZ; DMSO-d₆): −117.27, −74.89. MS [M+H]+=424.40

### Example 32

### Preparation of Compound 5001

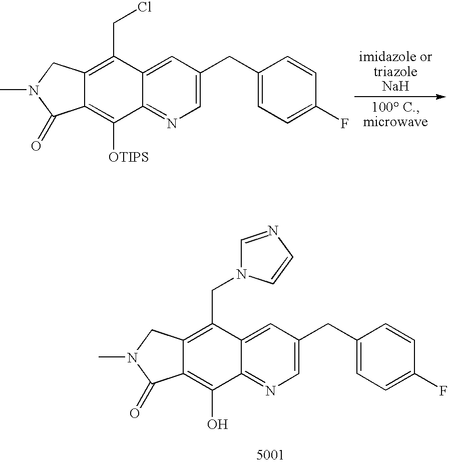

### General Procedure for the Synthesis of Benzylic Heterocycles 5001-5006

To a microwave vial containing 50 mg of the chloromethyl compound 5000 dissolved in 3 mL of a 1:1 solution of DMF/THF, was added 70 mg of the heterocyclic nucleophile. In the cases where triazole nucleophiles were employed, 50 mg of 60% NaH oil dispersion was added to the reaction mixture prior to heating. The reaction was heated at 100° C. for 20 minutes, at which time product formation, as well as C8-TIPS ether solvolysis, was seen to be complete as determined by LC/MS analysis of the reaction mixture. The mixture was quenched with 1 mL 1N HCl, and the resulting solution injected directly onto HPLC to yield the pure product as a light yellow powder after lyophilization.

Use of methylimidazole as nucleophile gave 9 mg 5003: ¹H NMR (400 MHz, d6-acetone) shows diagnostic peaks at δ (ppm): 8.80 (s, 1H), 8.20 (s, 1H) 4.56 (s, 2H) 4.28 (s, 2H), 3.15 (s, 3H) and 2.28 (s, 36H). MS 417 (M+H).

### Example 35

### Preparation of Compound 5004

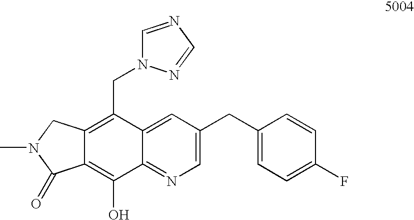

Compound 5004 was prepared following the procedure outlined in Example 32. Use of triazole in the general procedure gave 10 mg 5004: ¹H NMR (400 MHz, d6-acetone) shows diagnostic peaks at δ (ppm): 8.75 (s, 1H), 8.62, (s, 1H), 8.55 (s, 1H), 7.85 (s, 1H), 7.35 (m, 2H), 7.04 (m, 2H), 5.72 (s, 2H), 4.65 (s, 2H) 4.18 (s, 2H), 3.04 (s, 3H). MS=404 (M+H).

### Example 36

### Preparation of Compound 5005

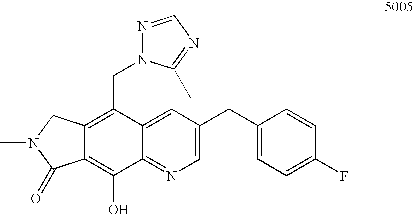

Compound 5005 was prepared following the procedure outlined in Example 32.

Use of methyltriazole as nucleophile gave 8 mg and 11 mg, respectively, of the regioisomeric triazoles 5005 and 5006. 5005: ¹H NMR (400 MHz, d₆-acetone) shows diagnostic peaks at δ (ppm): 8.81 (s, 1H), 8.33 (s, 1H), 7.70 (s, 1H), 7.30 (m, 2H), 7.13 (m, 2H), 5.61 (s, 1H), 4.51 (s, 2H) 4.16 (s, 2H), 3.02 (s, 3H). MS=418.1 (M+H).

### Example 37

### Preparation of Compound 5006

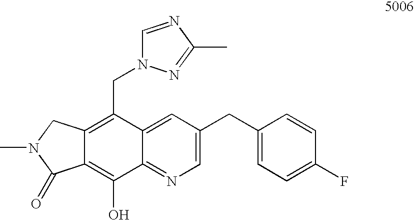

Compound 5006 was prepared following the procedure outlined in Example 32. Use of methyltriazole as nucleophile gave 8 mg and 11 mg, respectively, of the regioisomeric triazoles 5005 and 5006. 5006: ¹H NMR (400 MHz, d₆-acetone) shows diagnostic peaks at δ (ppm): 8.80 (s, 1H), 8.58 (s, 1H), 8.49 (s, 1H), 7.33 (m, 2H), 7.09 (m, 2H), 5.63 (s, 1H), 4.66 (s, 2H) 4.19 (s, 2H), 3.04 (s, 3H). MS=418.1 (M+H).

### Example 38

### Preparation of Compound 113

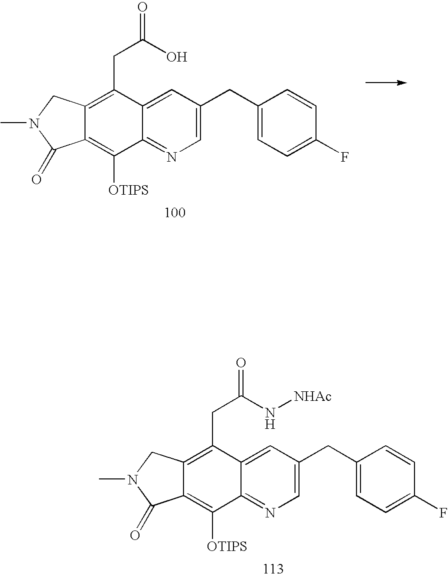

Compound 113 was prepared using procedures similar to those described herein. MS: 593.40 (M+1).

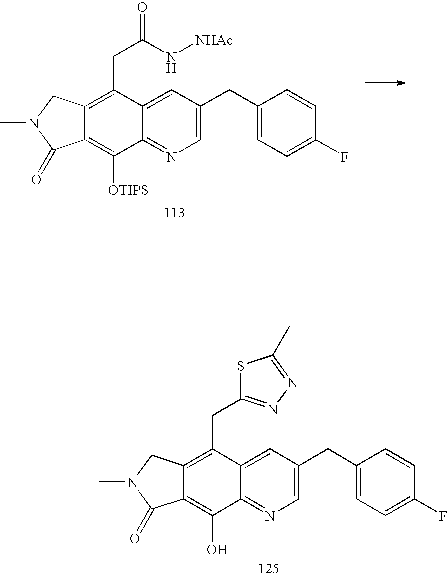

In a flask containing semi-carbazide 113 (55 mg, 0.093 mmol, 1 equiv.) in THF (3 mL, 0.025 M) was added Lawesson's Reagent (45 mg, 0.11 mol, 1.2 equiv) and heated to 75° C. Desilylation of TIPS also takes place during the reaction. Compound 125 was purified by HPLC. 400 MHz ¹H NMR ((DMSO-d₆) δ (ppm): 8.80 (s, 1H), 8.47 (s, 1H), 7.33-7.25 (m, 2H), 7.15-7.05 (m, 2H), 4.71 (s, 2H), 4.54 (s, 2H), 4.17 (s, 2H), 3.02 (s, 3H), 2.47 (s, 3H). ¹⁹F NMR ((DMSO-d₆) δ (ppm): −78.38, −117.85. MS: 435.35 (M+1).

### Example 39

### Preparation of Compound 122

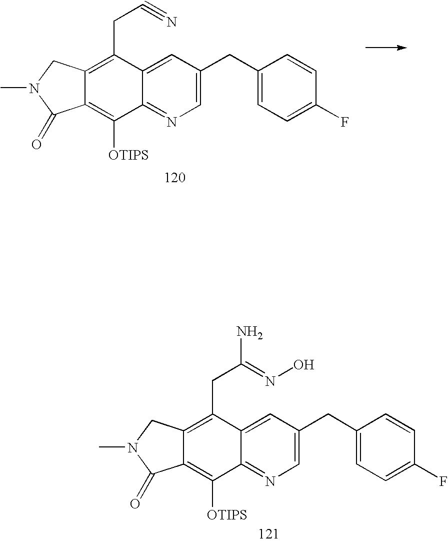

Nitrile 120 (100 mg, 0.20 mmol, 1 equiv.) which has previously been reported, was dissolved in EtOH (4 ml) followed by addition of NH₂OH (51 μL, 0.77 mmol, 4 equiv., 50% EtOH) before warming to 60° C. After the reaction was complete, it was diluted with ethyl acetate and quenched with water. The organic layer saturated was washed with NH₄Cl, aqueous LiCl, and brine, then dried (Na₂SO₄), filtered and concentrated down.

MS: 551.33 (M+1).

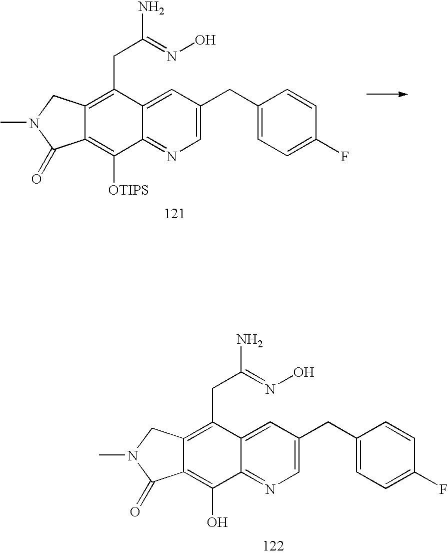

Compound 122 was prepared using procedures similar to those described herein. 400 MHz

¹H NMR ((DMSO-d₆) δ (ppm): 8.77 (d, J=1.6 Hz, 1H), 8.18 (s, 1H), 7.33-7.25 (m, 2H), 7.15-7.05 (m, 2H), 4.43 (s, 2H), 4.15 (s, 2H), 4.05 (s, 2H), 3.45-3.50 (bs, 2H), 2.98 (s, 3H). ¹⁹F NMR ((DMSO-d₆) δ (ppm): −74.14, −117.08. MS: 395.27 (M+1).

### Example 40

### Preparation of Compound 130

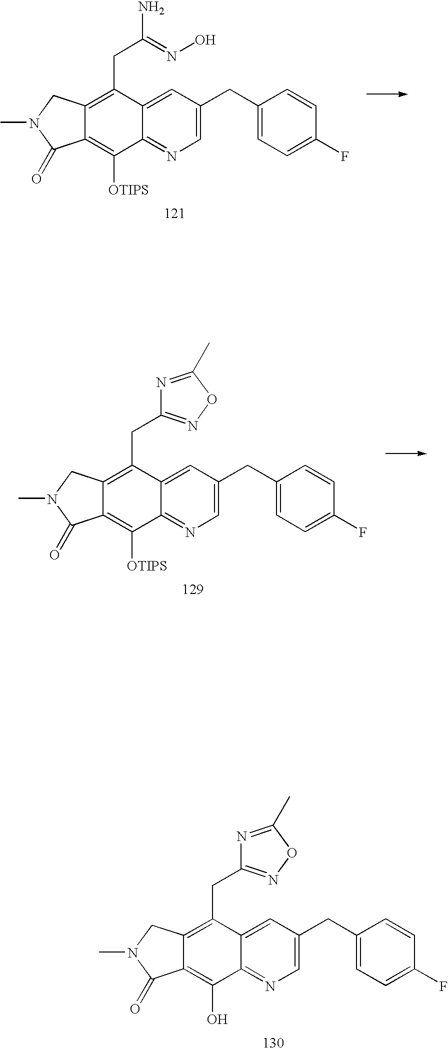

Compound 121 (88 mg, 0.07 mmol, 1 equiv.) was dissolved in THF (2 mL, 0.05 M) and DMF (0.5 mL) followed by addition of DIPEA (18 μL, 0.11 mmol, 1.5 equiv.) and AcCl (6 μL, 0.11 mmol, 1.5 equiv.) and stirred at room temperature. The reaction was concentrated in vacuo and dissolved in THF (2 ml) H₂0 (0.3 mL) and DIPEA (0.1 mL). After the cyclization, TFA (0.5 mL) was added and the reaction warmed to 50° C. The product was purified by HPC. 400 MHz ¹H NMR ((DMSO-d₆) δ (ppm): 8.75 (d, J=1.6 Hz, 1H), 8.39 (s, 1H), 7.39-7.34 (m, 2H), 7.15-7.05 (m, 2H), 4.51 (s, 2H), 4.32 (s, 2H), 4.14 (s, 2H), 2.99 (s, 3H), 2.34 (s, 3H). ¹⁹F NMR ((DMSO-d₆) δ (ppm): −79.33, −122.19

MS: 419.42 (M+1).

### Example 41

### Preparation of Compound 123

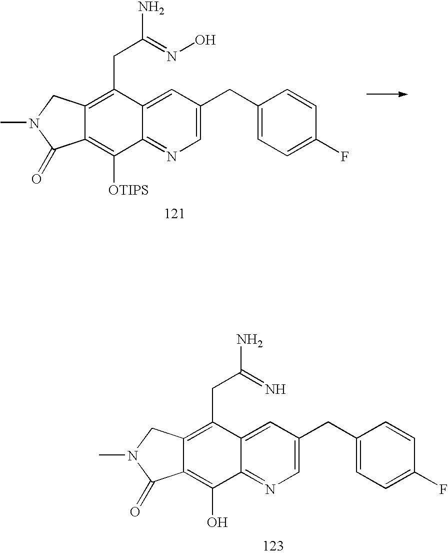

Amidoxime 121 (45 mg, 0.082 mmol, 1 equiv) was stirred in acetic acid (3 mL, 0.03 M) and to it added Pd (5 mg, 0.041 mmol, 0.5 equiv., 10% carbon) and ammonium formate (16 mg, 0.24 mmol, 3 equiv.). After the reaction was complete, the solids were filtered off over a pad of celite and the filtrate concentrated down. Amidine 123 was obtained after HPLC purification. 400 MHz ¹H NMR ((DMSO-d₆) δ (ppm): 8.76 (s, 1H), 8.42 (s, 1H), 7.51 (bs, 1 to H), 7.33-7.25 (m, 2H), 7.15-7.05 (m, 2H), 6.99 (bs, 2H), 4.49 (s, 2H), 4.18 (s, 2H), 3.73 (s, 2H), 3.02 (s, 3H). ¹⁹F NMR ((DMSO-d₆) δ (ppm): −74.49, −117.20 MS: 395.27 (M+1).

### Example 42

### Preparation of Compound 124

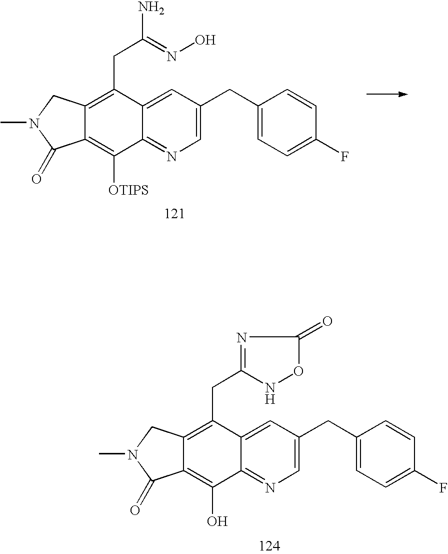

In a flask containing amidoxime 121 (40 mg, 0.075 mmol, 1 equiv.) in THF (3 mL) was added CDI (30 mg, 0.22 mol, 2.5 equiv) and heated to 70° C. Desilylation of TIPS also takes place during the reaction. Compound 124 was purified by HPLC.

400 MHz ¹H NMR ((DMSO-d₆) δ (ppm): 12.08 (bs, 1H), 8.76 (d, J=1.2 Hz, 1H), 8.31 (s, 1H), 7.33-7.25 (m, 2H), 7.15-7.05 (m, 2H), 4.49 (s, 2H), 4.18 (s, 2H), 4.14 (s, 2H), 2.99 (s, 3H).

¹⁹F NMR ((DMSO-d₆) δ (ppm): −74.38, −117.15

MS: 421.41 (M+1).

### Example 43

### Preparation of Compound 111

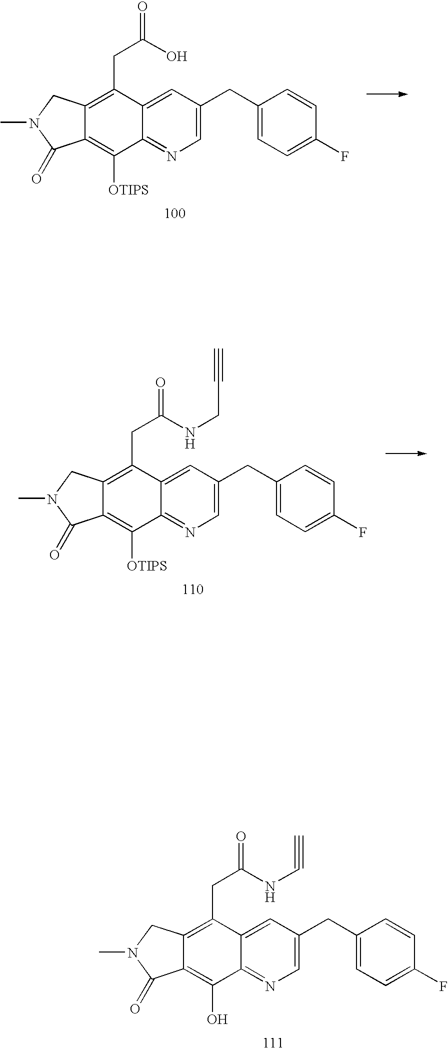

Compound 111 was made in manner similar to the example shown above. Compound 110 was not isolated or fully characterized but was carried on to the next reaction.

400 MHz ¹H NMR ((DMSO-d₆) δ (ppm): 8.74 (s, 1H), 8.52 (t, J=2.2 Hz, 1H), 8.39 (s, 1H), 7.37-7.28 (m, 2H), 7.25-7.18 (m, 2H), 4.46 (s, 2H), 3.76 (s, 2H), 3.75 (s, 2H), 2.99 (s, 3H). ¹⁹F NMR ((DMSO-d₆) δ (ppm): −77.39, −1118.65 (may not have been tuned/calibrated). MS: 418.43 (M+1).

### Example 44

### Preparation of Compound 112

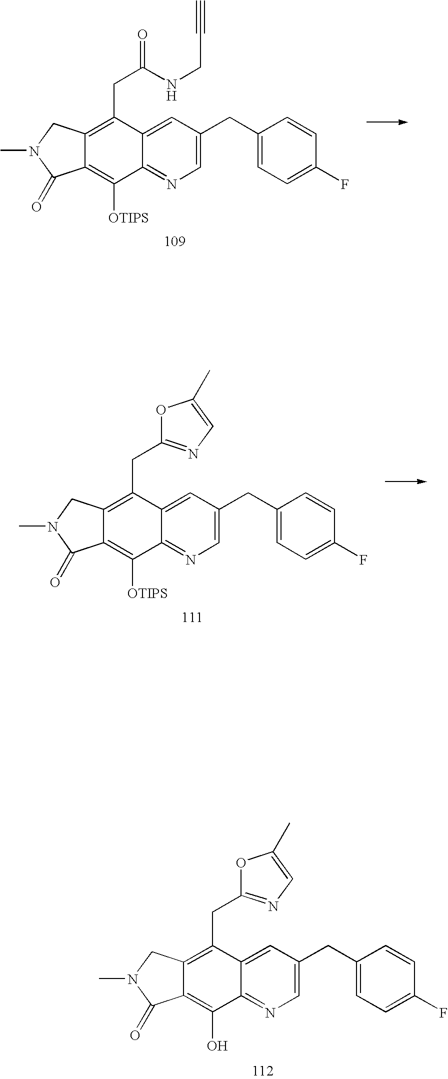

To a flask containing acetylene 109 (35 mg, 0.06 mmol, 1 equiv.) was added CH₂Cl₂ (3 ml, 0.01 M) followed by AuCl₃ (6 mg, 0.013 mmol, 0.3 equiv) and allowed to stir at room temperature. After the reaction was complete, it was concentrated in vacuo and dissolved in THF (5 mL) and water (2 mL) before adding TFA (500 μL). The desired compound was obtained by HPLC purification.

400 MHz ¹H NMR ((DMSO-d₆) δ (ppm): 8.80 (s, 1H), 8.44 (s, 1H), 7.40-7.29 (m, 2H), 7.15-7.08 (m, 2H), 6.86 (d, J=7.6 Hz, 1H), 4.52 (s, 2H), 4.36 (s, 2H), 4.18 (s, 2H), 3.03 (s, 3H), 2.12 (s, 3H). ¹⁹F NMR ((DMSO-d₆) δ (ppm): −74.78, −117.24

MS: 418.41 (M+1).

### Example 45

### Preparation of Compound 10

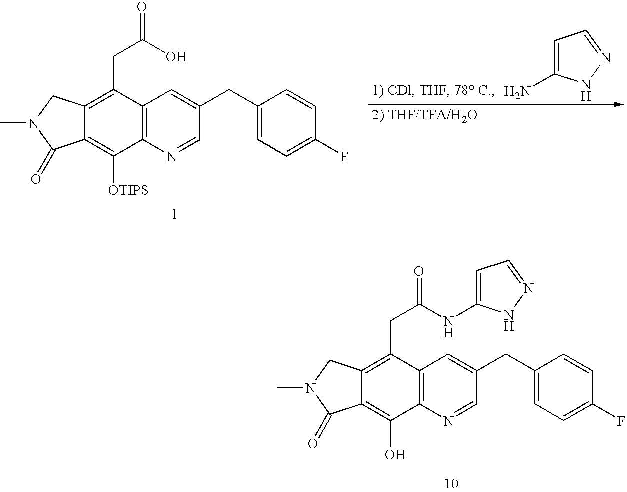

Heteroaryl C-5 amides (10-13) were synthesized from 1 and purified by flash chromatography. TIPS deprotection was accomplished using TFA. Compounds were purified by HPLC where necessary.

Compound 10: 2-[3-(4-Fluoro-benzyl)-9-hydroxy-7-methyl-8-oxo-7,8-dihydro-6H-pyrrolo[3,4-g]quinolin-5-yl]-N-(2H-pyrazol-3-yl)-acetamide. ¹H-NMR (300 MHZ; DMSO-d₆): d 10.84 (s, 1H), 8.82 (s, 1H), 8.59 (s, 1H), 7.58 (d, 1H), 7.36 (q, 2H), 7.01 (t, 2H), 6.37 (s, 1H), 4.57 (s, 2H), 4.19 (s, 2H), 4.02 (s, 2H), 3.06 (s, 3H). ¹⁹F-NMR (300 MHZ; DMSO-d₆): −117.18. MS [M+H]+=446.38

### Example 46

### Preparation of Compound 11

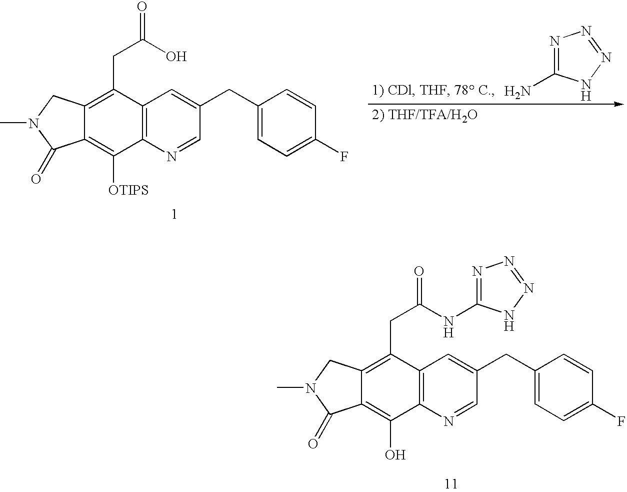

Compound 11 was prepared following the procedure outline in Example 45.

Compound 11: 2-[3-(4-Fluoro-benzyl)-9-hydroxy-7-methyl-8-oxo-7,8-dihydro-6H-pyrrolo[3,4-g]quinolin-5-yl]-N-(1H-tetrazol-5-yl)-acetamide, ¹H-NMR (300 MHZ; DMSO-d₆): d 12.40 (s, 1H), 10.36 br, 1H), 8.81 (s, 1H), 8.43 (s, 1H), 7.33 (s, 2H), 7.03 (t, 2H), 4.54 (s, 2H), 4.19 (s, 4H), 3.05 (s, 3H). ¹⁹F-NMR (300 MHZ; DMSO-d₆)-117.21-74.57 MS [M+H]+=448.21.

### Example 47

### Preparation of Compound 12

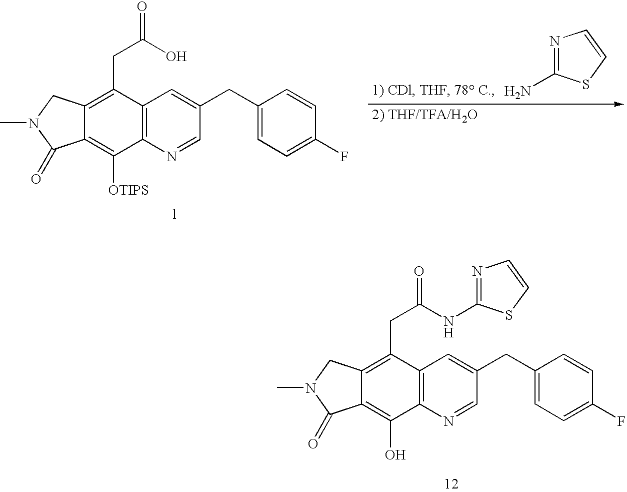

Compound 12 was prepared following the procedure outline in Example 45.

Compound 12: 2-[3-(4-Fluoro-benzyl)-9-hydroxy-7-methyl-8-oxo-7,8-dihydro-6H-pyrrolo[3,4-g]quinolin-5-yl]-N-thiazol-2-yl-acetamide. ¹H-NMR (400 MHZ; DMSO-d₆): d 12.50 (br, 1H), 8.93 (br, 1H), 8.79 (s, 1H), 8.43 (s, 1H), 7.49 (d, 1H), 7.31 (q, 2H), 7.15 (d, 1H), 6.96 (t, 2H), 4.53 (s, 2H), 4.16 (s, 2H), 4.13 (s, 2H), 3.03 (s, 3H).

¹⁹F-NMR (400 MHZ; DMSO-d₆): −117.07, −74.51. MS [M+H]+=463.23

### Example 48

### Preparation of Compound 13

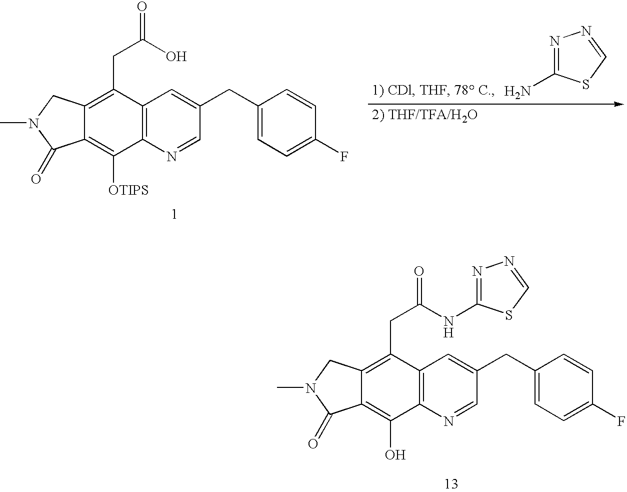

Compound 13 was prepared following the procedure outline in Example 45.

Compound 13: 2-[3-(4-Fluoro-benzyl)-9-hydroxy-7-methyl-8-oxo-7,8-dihydro-6H-pyrrolo[3,4-g]quinolin-5-yl]-N-[1,3,4]thiadiazol-2-yl-acetamide

¹H-NMR (300 MHZ; DMSO-d₆): d 12.94 (s, 1H), 10.30 (br, 1H), 9.16 (s, 1H), 8.82 (s, 1H), 8.41 (s, 1H), 7.32 (t, 2H), 7.01 (t, 2H), 4.55 (s, 3H), 4.19 (d, 4H), 3.05 (s, 3H). ¹⁹F-NMR (300 MHZ; DMSO-d₆): −117.30, −74.87. MS [M+H]+=464.23

### Example 49

### Preparation of Compound 9

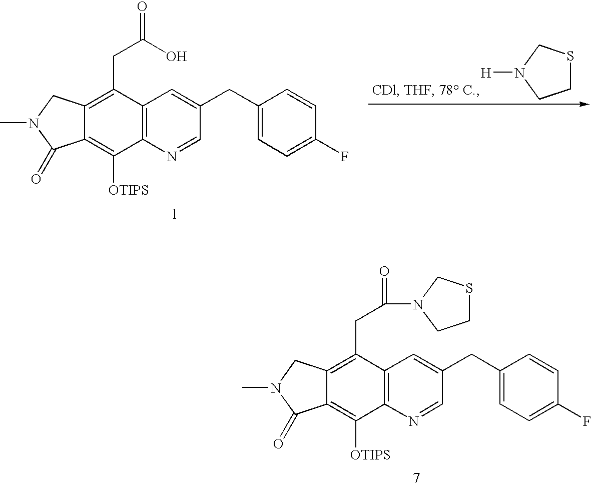

Compound 7 was prepared from the corresponding acid and purified by flash chromatography. It was found that compound 7 was unstable to air oxidation and therefore it was used directly to prepare compounds 8 and 9.

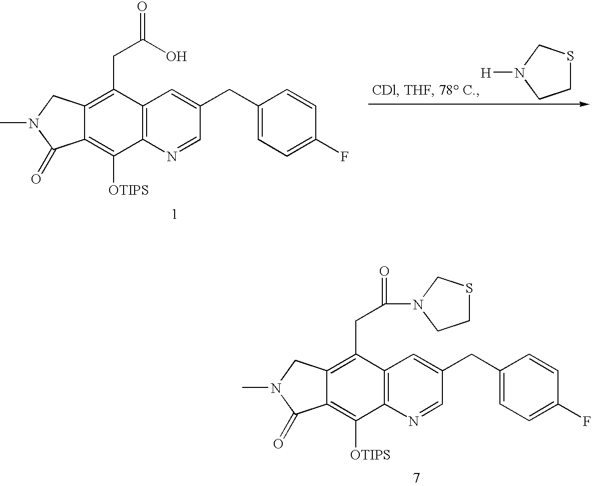

### Compound 73-(4-Fluoro-benzyl)-7-methyl-5-(2-oxo-2-thiazolidin-3-yl-ethyl)-9-triisopropylsilanyloxy-6,7-dihydro-pyrrolo[3,4-g]quinolin-8-one

MS [M+H]+=608.36. Purified compound 7 was dissolved in MeOH (0.01M) and to this was added 4 equivalents of oxone (0.1M in H₂O) portionwise periodically and allowed to stir at 40° C. Reaction progress was monitored by LCMS and when compound 7 was converted into equal proportions of 8 and 9 oxone addition was ceased and reaction solvents removed in vacuo, residue dissolved in DMF, filtered and purified by HPLC affording pure 8 and 9.

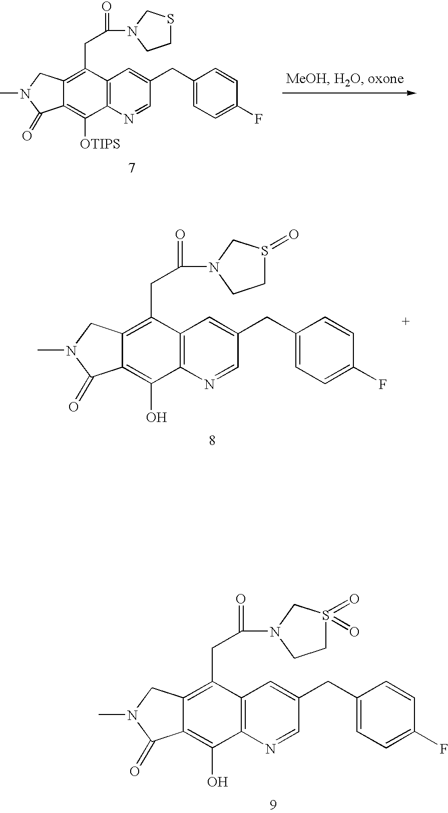

Compound 8: 3-(4-Fluoro-benzyl)-9-hydroxy-7-methyl-5-[2-oxo-2-(1-oxo-114-thiazolidin-3-yl)-ethyl]-6,7-dihydro-pyrrolo[3,4-g]quinolin-8-one. ¹H-NMR (400 MHZ; DMSO-d₆): d 8.72 (s, 1H), 8.22 (s, 1H), 7.29 (t, 2H), 7.06 (m, 2H) 5.08 (d, 1H), 4.62 (d, 1H) 4.62 (d, 1H), 4.49 (d, 2H) 4.36 (d, 2H), 4.28-4.28 (m, 2H), 4.12 (d, 4H), 3.95 (d, 1H), 2.98 (s, 3H)

¹⁹F-NMR (400 MHZ; DMSO-d₆): −117.24-73.93. MS [M+H]+=468.32

Compound 9: 5-[2-(1,1-Dioxo-116-thiazolidin-3-yl)-2-oxo-ethyl]-3-(4-fluoro-benzyl)-9-hydroxy-7-methyl-6,7-dihydro-pyrrolo[3,4-g]quinolin-8-on ¹H-NMR (400 MHZ; DMSO-d₆): d 8.72 (s, 1H), 8.22 (s, 1H), 7.28 (q, 2H), 7.07 (d, 2H) 4.95 (s, 1H), 4.24 (s, 1H), 4.35 (d, 2H), 4.22 (t, 1H), 4.13 (s, 2H), 4.11 (s, 1H), 3.79 (t, 1H), 3.59 (t, 1H), 3.42 (t, 1H), 2.98 (s, 3H). ¹⁹F-NMR (400 MHZ; DMSO-d₆): −117.22, −74.97. MS [M+H]+=484.37

### Example 50

### Preparation of Compound 5

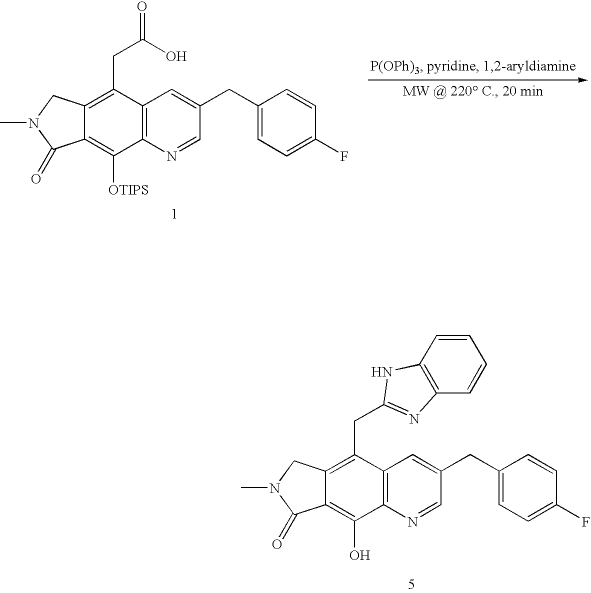

To a conical-bottomed microwave vial was added 1 (54 mg, 0.1 mmol), 1,2-aryldiamine (0.1 mmol) triphenyl phosphine (35 μL, 0.12 mmol) and anhydrous pyridine (0.5 mL, 0.2M). The sealed vial was irradiated in the microwave for 20 min at 220° C. Reaction was cooled then transferred to a round-bottomed flask and solvents removed in vacuo. Compounds were purified by HPLC. Lin, S.-Y.; Isome, Y.; Steward, E.; Liu, J.-F.; Yohannes, D.; Yu, Libing. Tett. Lett. 2006, 47, 2883-2886. Compound 5: 5-(1H-Benzoimidazol-2-ylmethyl)-3-(4-fluoro-benzyl)-9-hydroxy-7-methyl-6,7-dihydro-pyrrolo[3,4-g]quinolin-8-one. ¹H-NMR (400 MHZ; DMSO-d₆): δ 8.81 (d, 1H), 8.23 (s, 1H), 7.62 (q, 2H), 7.45 (q, 2H), 7.15 (q, 2H), 6.87 (t, 2H), 4.83 (s, 2H), 4.58 (s, 2H), 4.09 (s, 2H), 3.02 (s, 3H); ¹⁹F-NMR (400 MHZ; DMSO-d₆): −74.76, −116.97 MS [M+H]+=453.36

### Example 51

### Preparation of Compound 6

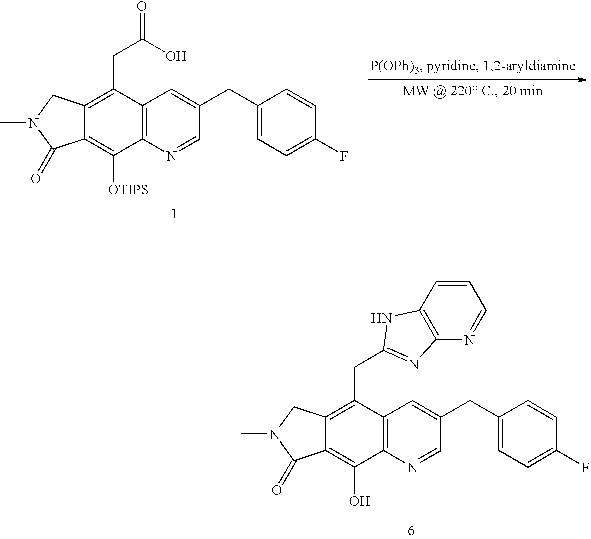

Compound 6 was prepared by the method outlined in Example 50.

Compound 6: 3-(4-Fluoro-benzyl)-9-hydroxy-5-(1H-imidazo[4,5-b]pyridin-2-ylmethyl)-7-methyl-6,7-dihydro-pyrrolo[3,4-g]quinolin-8-one. ¹H-NMR (400 MHZ; DMSO-d₆): δ 8.79 (d, 1H), 8.42 (d, 1H), 8.39 (s, 1H), 8.07 (d, 1H) 7.38 (q, 1H), 7.20 (q, 2H), 6.91 (t, 2H), 4.68 (s, 2H), 4.60 (s, 2H), 4.11 (s, 2H), 3.03 (s, 3H); ¹⁹F-NMR (400 MHZ; DMSO-d₆): −75.08, −117.06. MS [M+H]+=454.40

### Example 52

### Preparation of Compound 9

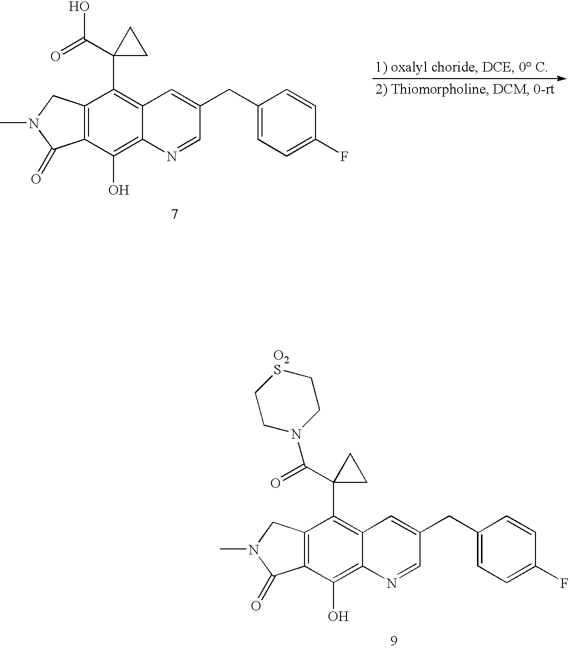

The scaffold 7 (1 eq, 0.2 mmol) was suspended in DCE (5 mL) and 5 drop DMF added. Reaction mixture cooled to 0° C. in ice bath and placed under nitrogen atmosphere. Oxalyl chloride (10 eq, 2.0 mmol) was added via syringe and the reaction mixture stirred at ambient temperature for 15 min. LC/MS indicated conversion to the acid chloride was complete. The reaction mixture was diluted with DCE (10 mL) and concentrated, then azeotroped with dry THF (20 mL). The residue was re-suspended in DCM (5 mL) and the amine (3 eq, 0.6 mmol) added via syringe. The reaction mixture continued at room temperature and LC/MS after 1 h showed the amide formation was complete. The reaction mixture was diluted with ethyl acetate and quenched with 1N HCl. The layers were separated and the organics washed with brine. The solvent was dried over sodium sulfate and concentrated to a red-brown residue. The residue was re-dissolved in 2:1 DMSO:MeOH and purified by reverse phase HPLC (ACN/H₂O) to afford the desired amide 9. 300 MHz ¹H NMR (Acetone-d₆) δ (ppm): 8.9 (s, 1H), 8.7 (s, 1H), 7.4 (t, 2H), 7.1 (t, 2H), 4.8 (s, 2H), 4.2 (s, 2H), 3.8 (m, 4H), 3.1 (s, 3H), 2.6 (m, 4H), 2.0 (s, 4H). m/z 524 (M+H).

### Example 53

### Preparation of Compound 18

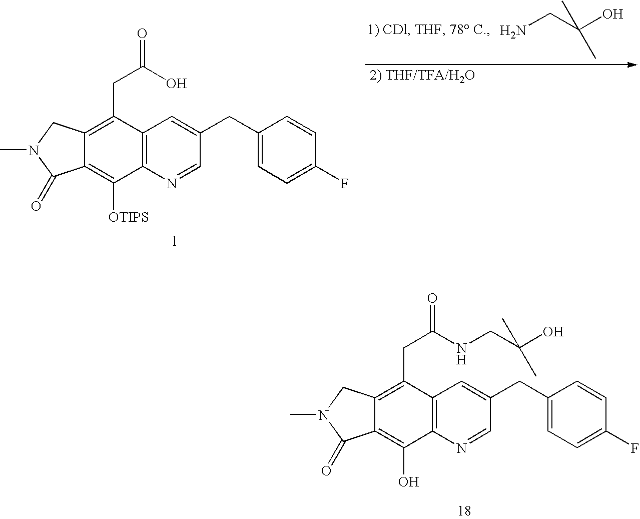

Compound 18 was synthesized from 1 and purified by flash chromatography before subjecting to TFA promoted TIPS removal. Compound 18: 2-[3-(4-Fluoro-benzyl)-9-hydroxy-7-methyl-8-oxo-7,8-dihydro-6H-pyrrolo[3,4-g]quinolin-5-yl]-N-(2-hydroxy-2-methyl-propyl)-acetamide. ¹H-NMR (400 MHZ; DMSO-d₆): δ 8.76 (s, 1H), 8.40 (s, 1H), 7.67 (s, 1H), 7.33 (t, 2H) 7.09 (t, 2H), 4.78 (br, 1H), 4.49 (s, 2H), 4.16 (s, 2H), 3.73 (s, 2H), 3.32 (s, 2H), 3.02 (s, 3H), 1.11 (s, 3H). ¹⁹F-NMR (400 MHZ; DMSO-d₆): −117.26, −73.83 MS [M+H]+=452.48

### Example 54

### Preparation of Compound 8a

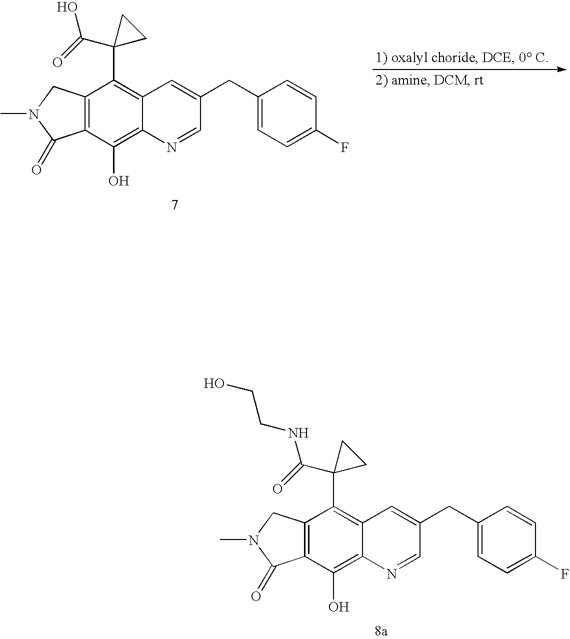

The scaffold 7 (1 eq, 0.2 mmol) was suspended in DCE (5 mL) and 5 drop DMF added. Reaction mixture cooled to 0° C. in ice bath and placed under nitrogen atmosphere. Oxalyl chloride (10 eq, 2.0 mmol) was added via syringe and the reaction mixture stirred at ambient temperature for 15 min. LC/MS indicated conversion to the acid chloride was complete. The reaction mixture was diluted with DCE (10 mL) and concentrated, then azeotroped with dry THF (20 mL). The residue was re-suspended in DCM (5 mL) and the amine (3 eq, 0.6 mmol) added via syringe. The reaction mixture continued at room temperature and LC/MS after 1 h showed the amide formation was complete. The reaction mixture was diluted with ethyl acetate and quenched with 1N HCl. The layers were separated and the organics washed with brine. The solvent was dried over sodium sulfate and concentrated to a red-brown residue. The residue was re-dissolved in 2:1 DMSO:MeOH and purified by reverse phase HPLC (ACN/H₂O) to afford the desired amide 8a. 400 MHz ¹H NMR (DMSO-d₆) δ (ppm) 8.8 (s, 1H), 8.3 (s, 1H), 7.3 (t, 2H), 7.1 (t, 2H), 5.7 (t, 1H), 4.7 (d, 1H), 4.3 (d, 1H), 4.2 (s, 2H), 3.2 (m, 2H), 3.1 (m, 2H), 3.0 (s, 3H), 1.8 (t, 1H)₃, 1.4 (t, 1H), 1.2 (t, 1H), 0.8 (t, 1H). m/z=450 (M+H).

### Example 55

### Preparation of Compound 8b

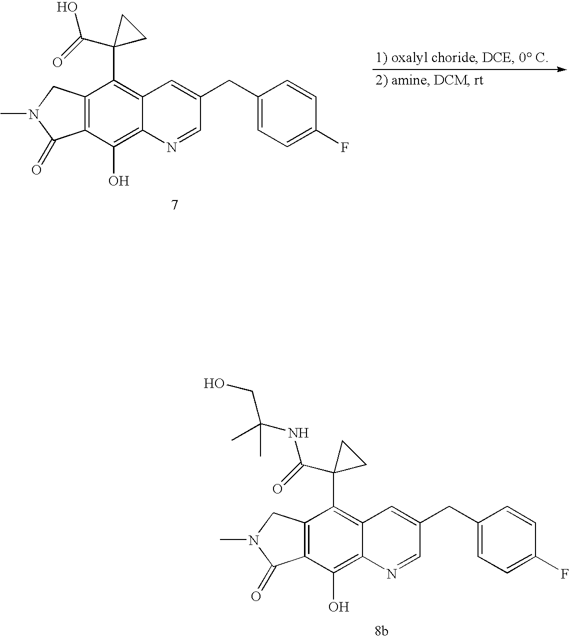

Compound 8b was prepared following the method outlined in Example 54.

8b—400 MHz ¹H NMR (DMSO-d₆) δ (ppm); 8.8 (s, 1H), 8.3 (s, 1H), 7.3 (t, 2H), 7.1 (t, 2H), 5.7 (t, 1H), 4.7 (d, 1H), 4.3 (d, 1H), 4.2 (s, 2H), 3.1 (s, 3H), 2.9 (t, 2H), 2.8 (t, 2H), 1.6 (t, 1H), 1.4 (t, 1H), 1.1 (t, 1H), 1.0 (s, 3H), 0.9 (s, 3H), 0.8 (t, 1H). m/z 478 (M+H).

### Example 56

### Preparation of Compound 16

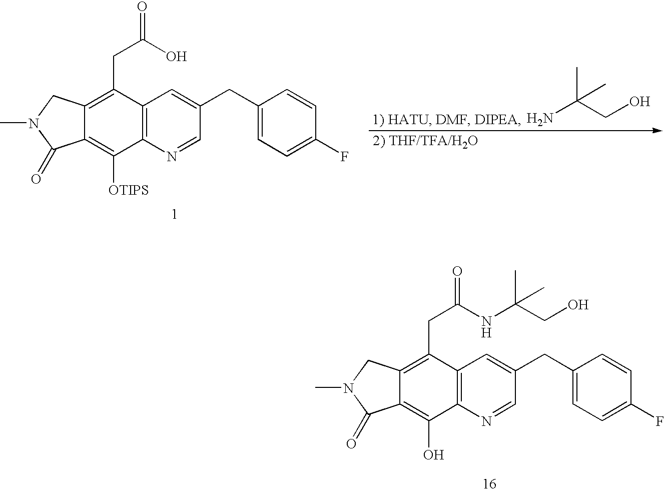

Compound 16 was synthesized from 1 and purified by flash chromatography before subjecting to TFA promoted TIPS removal. Compound 16: 2-[3-(4-Fluoro-benzyl)-9-hydroxy-7-methyl-8-oxo-7,8-dihydro-6H-pyrrolo[3,4-g]quinolin-5-yl]-N-(2-hydroxy-1,1-dimethyl-ethyl)-acetamide. ¹H-NMR (400 MHZ; DMSO-d₆): δ 8.74 (s, 1H), 8.44 (s, 1H), 7.98 (s, 1H), 7.31 (t, 2H) 7.09 (t, 2H), 4.51 (s, 2H), 4.42 (s, 1H), 4.16 (s, 2H), 3.82 (s, 2H), 3.02 (s, 3H), 0.96 (s, 6H). ¹⁹F-NMR (400 MHZ; DMSO-d₆): −117.18, −73.80 MS [M+H]+=452.46.

### Example 57

### Preparation of Compound 17

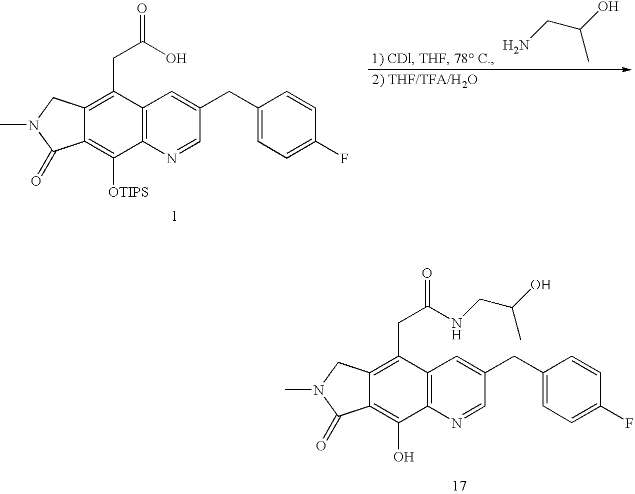

Compound 17 was synthesized from 1 and purified by flash chromatography before subjecting to TFA promoted TIPS removal. Compound 17: 2-[3-(4-Fluoro-benzyl)-9-hydroxy-7-methyl-8-oxo-7,8-dihydro-6H-pyrrolo[3,4-g]quinolin-5-yl]-N-(2-hydroxy-propyl)-acetamide. ¹H-NMR (400 MHZ; DMSO-d₆): δ 8.76 (s, 1H), 8.42 (s, 1H), 8.06 (d, 1H), 7.33 (q, 2H) 7.10 (t, 2H), 4.64 (d, 1H), 4.50 (s, 2H), 4.16 (s, 2H), 3.78 (s, 2H), 3.58 (t, 1H), 3.02 (s, 3H), 2.95 (q, 2H), 0.92 (d, 3H). ¹⁹F-NMR (400 MHZ; DMSO-d₆): −117.24, −73.83. MS [M+H]+=438.46

### Example 58

The following illustrate representative pharmaceutical dosage forms, containing a compound of formula I, II, or III (‘Compound X’), for therapeutic or prophylactic use in humans.

| (i) Tablet 1 | mg/tablet |
|---|---|
| Compound X = | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |
| (ii) Tablet 2 | mg/tablet |
| Compound X = | 20.0 |
| Microcrystalline cellulose | 410.0 |
| Starch | 50.0 |
| Sodium starch glycolate | 15.0 |
| Magnesium stearate | 5.0 |
| | 500.0 |
| (iii) Capsule | mg/capsule |
| Compound X = | 10.0 |
| Colloidal silicon dioxide | 1.5 |
| Lactose | 465.5 |
| Pregelatinized starch | 120.0 |
| Magnesium stearate | 3.0 |
| | 600.0 |
| (iv) Injection 1 (1 mg/ml) | mg/ml |
| Compound X = (free acid form) | 1.0 |
| Dibasic sodium phosphate | 12.0 |
| Monobasic sodium phosphate | 0.7 |
| Sodium chloride | 4.5 |
| 1.0 N Sodium hydroxide solution | q.s. |
| (pH adjustment to 7.0-7.5) | |
| Water for injection | q.s. ad 1 mL |
| (v) Injection 2 (10 mg/ml) | mg/ml |
| Compound X = (free acid form) | 10.0 |
| Monobasic sodium phosphate | 0.3 |
| Dibasic sodium phosphate | 1.1 |
| Polyethylene glycol 400 | 200.0 |
| 01 N Sodium hydroxide solution | q.s. |
| (pH adjustment to 7.0-7.5) | |
| Water for injection | q.s. ad 1 mL |
| (vi) Aerosol | mg/can |
| Compound X = | 20.0 |
| Oleic acid | 10.0 |
| Trichloromonofluoromethane | 5,000.0 |
| Dichlorodifluoromethane | 10,000.0 |
| Dichlorotetrafluoroethane | 5,000.0 |

The above formulations may be obtained by conventional procedures well known in the pharmaceutical art.

All publications, patents, and patent documents are incorporated by reference herein, as though individually incorporated by reference. The above description is not intended to detail all modifications and variations of the invention. It will be appreciated by those skilled in the art that changes can be made to the embodiments described above without departing from the inventive concept. It is understood, therefore, that the invention is not limited to the particular embodiments described above, but is intended to cover modifications that are within the spirit and scope of the invention, as defined by the language of the following claims.

1. A compound of formula (I): 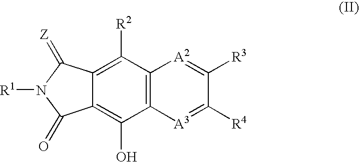
wherein:
A² is N or CRₐ;
A³ is N or CRₐ;
R¹ is H, R_{b}, or -Q-R_{c};
R² is C₁-C₆alkoxycarbonyl, —C(═O)C(═O)ORₐ, or —C(═O)NRₐR_{g};
or R² is C₁-C₆alkyl that is substituted with one or more groups independently selected from heterocycle, substituted heterocycle, —C(═O)ORₐ, C(═N—ORₐ)—NRₑRₑ, —C(═NRₐ)—NRₑRₑ, —P(═O)(Rₙ)(Rₙ), —C(═O)N(Rₑ)NRₑRₑ, —C(═O)NRₐR_{g}, and —C(═O)Rₕ;
or R² is C₃-C₈carbocycle that is substituted with one or more groups independently selected from heterocycle, C(═O)ORₐ, —C(═O)NRₑRₑ, —C(═O)N(Rₐ)—S(O)₂Rₐ, —C(═O)R_{b}, 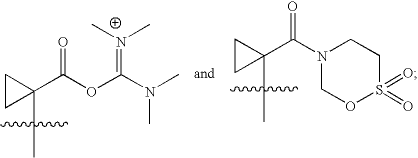
R³ is H, halo, or C₁-C₆alkyl that is optionally substituted with Rₖ;
R⁴ is H, halo, or C₁-C₆alkyl that is optionally substituted with Rₖ;
Q is C₁-C₆alkylene;
Z is O or two hydrogens;
each Rₐ is independently H or C₁-C₆alkyl;
R_{b} is C₁-C₆alkyl, C₂-C₆alkenyl, or C₁-C₆alkynyl, each of which is optionally substituted with one or more halo, hydroxy, C₁-C₆alkoxy, dimethylamino, diethylamino, N-ethyl-N-methylamino, morpholino, thiomorpholino, piperidino, or piperazino;
R_{c} is a C₃-C₁₂-carbocycle, a substituted C₃-C₁₂-carbocycle, aryl, substituted aryl, heteroaryl, or substituted heteroaryl;
each R_{d} is independently C₁-C₆alkyl;
each Rₑ is independently H, C₁-C₆alkyl, C₁-C₆alkoxy, C₂-C₆alkenyl, C₂-C₆alkynyl, C₃-C₁₂-carbocycle, wherein each C₁-C₆alkyl, C₁-C₆alkoxy, C₂-C₆alkenyl, C₃-C₁₂-carbocycle, and C₂-C₆alkynyl of Rₑ is optionally substituted with aryl, heteroaryl, substituted aryl, substituted heteroaryl, cyano, hydroxy, C₃-C₁₂-carbocycle, —C(═O)ORₐ, —C(O)C(═O)ORₐ, —C(═O)NR_{g}R_{g}, —N(Rₐ)—C(═O)—Rₐ, —N(Rₐ)—S(O)₂—Rₐ heteroaryl, —S(O)₂—NR_{g}R_{g}, —C(═NRₘ)—NR_{g}R_{g}, or —C(═O)NR_{g}R_{g};
each R_{f} is independently H, C₁-C₆alkyl, phenyl, or phenylC₁-C₆alkyl, wherein any phenyl ring of R_{f} is optionally substituted with one or more fluoro, chloro, bromo, iodo, cyano, C₁-C₆alkyl, C₁-C₆alkyl-C(═O)—, C₁-C₆alkyl-S(O)₂—, —C(═O)NRₐRₐ, or —C═O)ORₐ;
each R_{g} is independently —S(O)₂—Rₐ, heterocycle, substituted heterocycle, C₂-C₆alkynyl or each R_{g} is C₁-C₆alkyl or C₃-C₁₂-carbocycle, which C₁-C₆alkyl or C₃-C₁₂-carbocycle is substituted with one or more —C(═O)ORₐ, or —S(O)₂—NRₐRₐ;
each Rₕ is independently selected from: 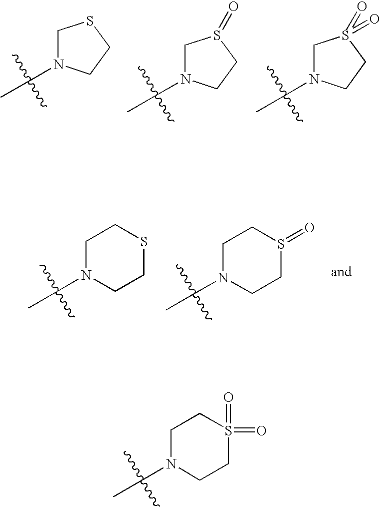
each Rₖ is phenyl, optionally substituted with one or more F, Cl, Br, I, hydroxy, cyano, trifluoromethyl, trifluoromethoxy, or C₁-C₆alkyl; and
each Rₘ is hydrogen, hydroxy, C₁-C₆alkyl, or C₁-C₆alkoxy;
each Rₙ is independently H, C₁-C₆alkyl, phenyl, phenylC₁-C₆alkyl, C₁-C₆alkoxy, phenoxy, or phenylC₁-C₆alkoxy, wherein any phenyl ring of Rₙ is optionally substituted with one or more groups independently selected from fluoro, chloro, bromo, iodo, cyano, C₁-C₆alkyl, C₁-C₆alkyl-C(O)—, C₁-C₆alkyl-S(O)₂—, —C(═O)NRₐRₐ, and —C(═O)ORₐ;
or a pharmaceutically acceptable salt or prodrug thereof.

2. The compound of claim 1 wherein A² is N.

3. The compound of claim 1 wherein A² is CRₐ.

4. The compound of claim 1 wherein A³ is N.

5. The compound of claim 1 wherein A³ is CRₐ;

6. The compound of claim 1 wherein R¹ is H.

7. The compound of claim 1 wherein R¹ is R_{b}

8. The compound of claim 1 wherein R¹ is -Q-R_{c}.

9. The compound of claim 1 wherein R¹ is methyl.

10. The compound of claim 1 wherein R² is C₁-C₆alkoxycarbonyl, —C(═O)C(═O)ORₐ, or —C(═O)NRₐR_{g}.

11. The compound of claim 1 wherein R² is C₁-C₆alkyl that is substituted with one or more groups independently selected from heterocycle, substituted heterocycle, —C(═O)ORₐ, —C(═N—ORₐ)—NRₑRₑ, —C(═NRₐ)—NRₑRₑ, —P(═O)(Rₙ)(Rₙ), —C(═O)N(Rₑ)NRₑRₑ, —C(═O)NRₐR_{g}, and —C(═O)Rₕ.

12. The compound of claim 1 wherein R² is C₃-C₈carbocycle that is substituted with one or more groups independently selected from heterocycle, —C(═O)ORₐ, —C(═O)NRₑRₑ, —C(═O)N(Rₐ)—S(O)₂Rₐ,

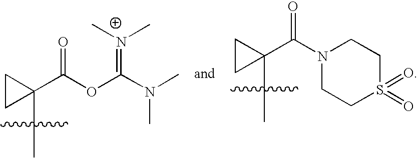

13. The compound of claim 1 wherein R² is C₁-C₆alkoxy that is substituted with

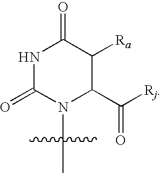

14. The compound of claim 1 wherein R² is methoxycarbonyl, oxalo, N-(1-carboxycyclopropyl)aminocarbonyl, N-(2-carboxy-2-methylethyl)aminocarbonyl, or methylsulfonylaminocarbonyl.

15. The compound of claim 1 wherein R² is N-(1-carboxycyclopropyl)aminocarbonylmethyl, N-(2-carboxy-2-methylethyl)aminocarbonylmethyl, tetrazoylmethyl, methylsulfonylaminocarbonylmethyl, methoxycarbonylmethyl, diethylphosphonylmethyl, 2,6-difluorobenzylphosphinylmethyl, 1,1-dioxothiomorpholinocarbonylmethyl, N-(2-aminosulfonylethyl)aminocarbonylmethyl, 5-methyl, 1,3,4-oxadiazolylmethyl, 5-(1-methylamino)-1,3,4-oxadiazolylmethyl, hydrazinocarbonylmethyl, imidazolyl, 5-amino-1,3,4-oxadiazolylmethyl, tetrazolylaminocarbonylmethyl, 1-benzimidazolylmethyl, 1,2,4-triazolylmethyl, 1-methyltetrazolylmethyl, 2-methyl-1,3,4-thiadiazolylmethyl, 2-methylimidazolylmethyl, 5-methyl-1,2,4-triazolylmethyl, 3-methyl-1,2,4-triazolylmethyl, 5-methyl-1,2,4-oxadiazolylmethyl, hydroxyamidinomethyl, amidinomethyl, 1,2-dihydro-5-oxa-1,3,4-oxadiazolylmethyl, 2-propynylaminocarbonylmethyl, 5-methyl-1,3-oxadiazolylmethyl, 2-benzimidazolylmethyl, N-(1,3,4-thiadiazol-2-yl)aminocarbonylmethyl, N-(5-pyrazolyl)aminocarbonylmethyl

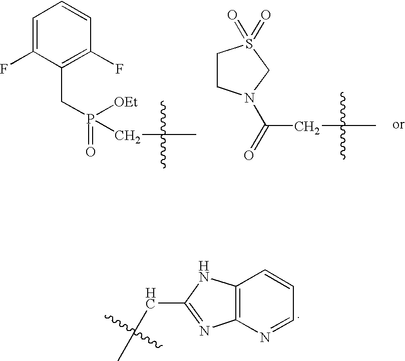

16. The compound of claim 1 wherein R² is 1-carboxycyclopropyl, 1-methoxycarbonylcyclopropyl, 1-(aminocarbonyl)cyclopropyl, 1-(methylsulfonulaminocarbonyl)cycloprppyl, 1-(N-methylaminocarbonyl)cyclopropyl, 1-N-(2-hydroxy-1,1dimethylethyl)aminocarbonyl)cyclopropyl, 1-(N-(2-hydroxyethyl)aminocarbonyl)cyclopropyl,

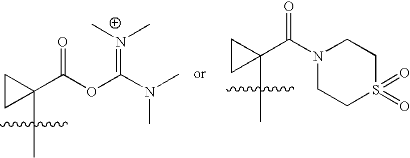

17. The compound of claim 1 wherein R² is 1-carboxycyclopropyl.

18. The compound of claim 1 wherein R² is 1-methoxycarbonylcyclopropyl.

19. The compound of claim 1 wherein R³ is H.

20. The compound of claim 1 wherein R³ is halo.

21. The compound of claim 1 wherein R³ is C₁-C₆alkyl that is optionally substituted with Rₖ.

22. The compound of claim 1 wherein R³ is C₁-C₆alkyl that is substituted with Rₖ.

23. The compound of claim 1 wherein R³ is 4-fluorobenzyl, or 4,6-difluoro-3-chlorobenzyl.

24. The compound of claim 1 wherein R⁴ is H.

25. The compound of claim 1 wherein R⁴ is halo.

26. The compound of claim 1 wherein R⁴ is C₁-C₆alkyl that is optionally substituted with Rₖ.

27. The compound of clam 1 wherein R⁴ is hydrogen.

28. The compound of claim 1 wherein Z is two hydrogens.

29. The compound 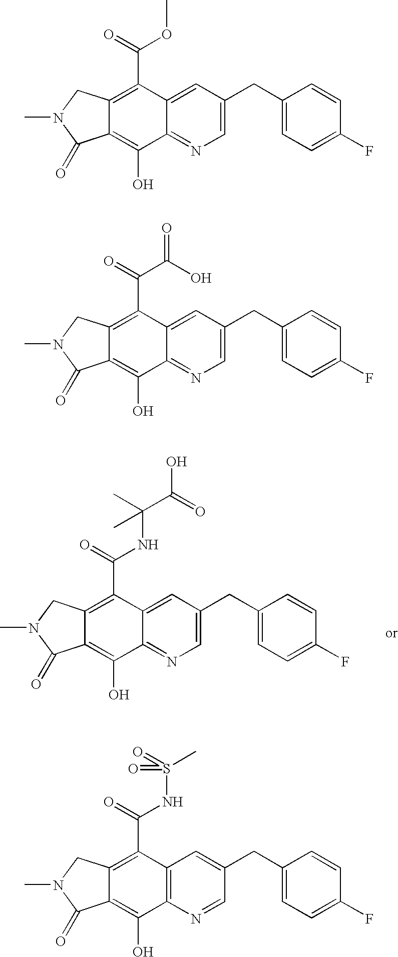
or a pharmaceutically acceptable salt or prodrug thereof.

30. The compound 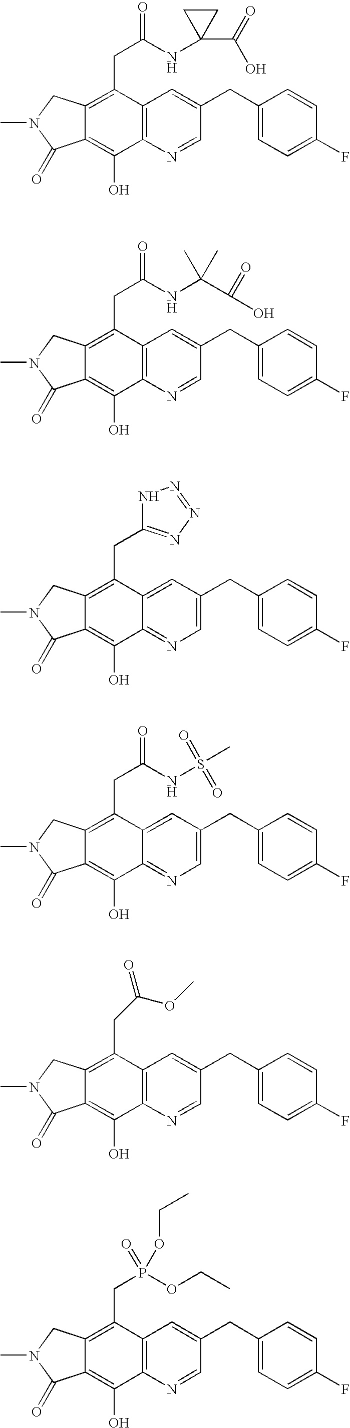 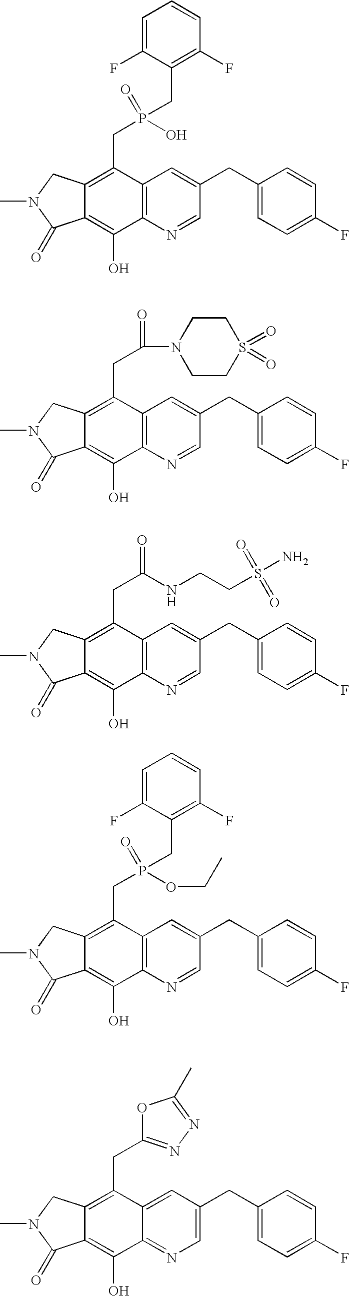 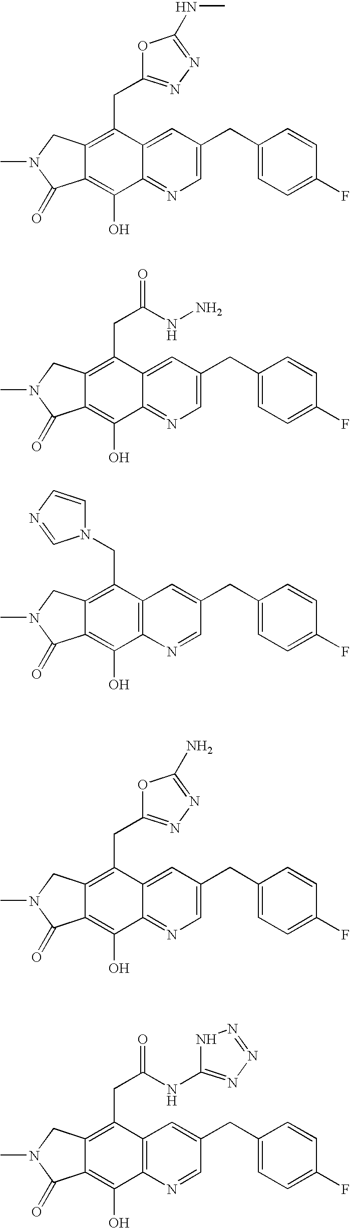 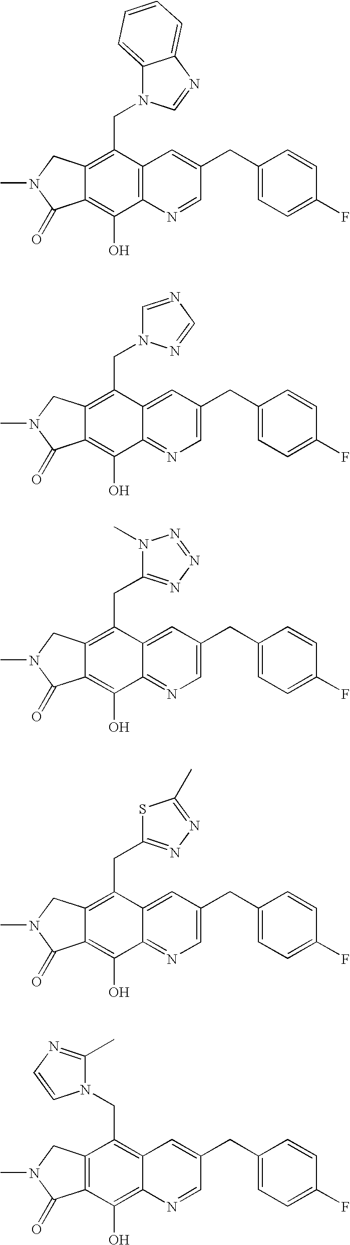 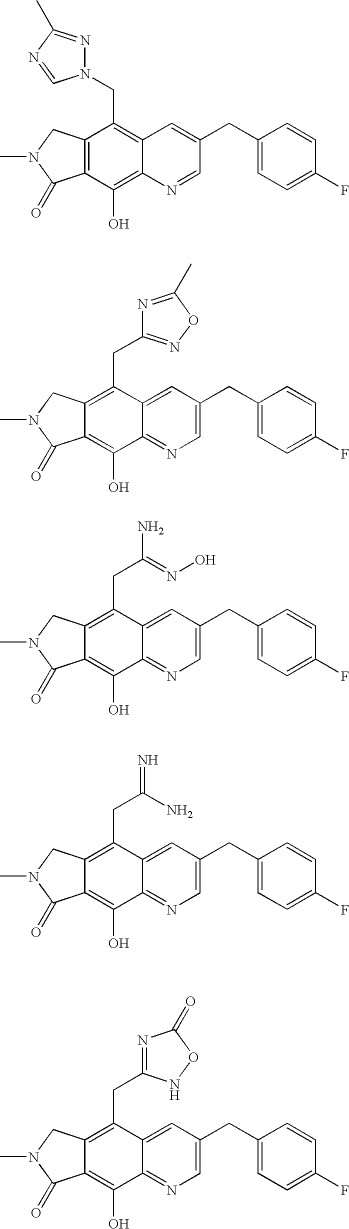 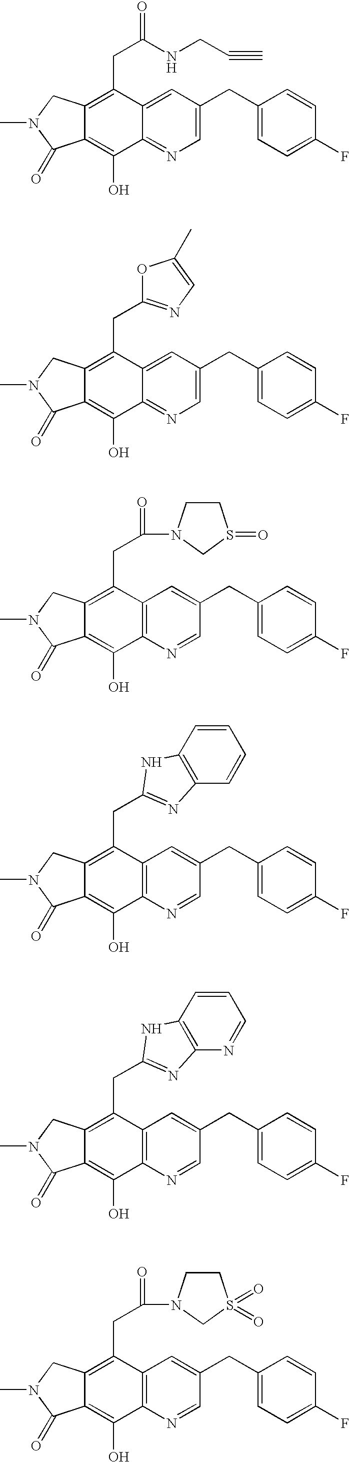 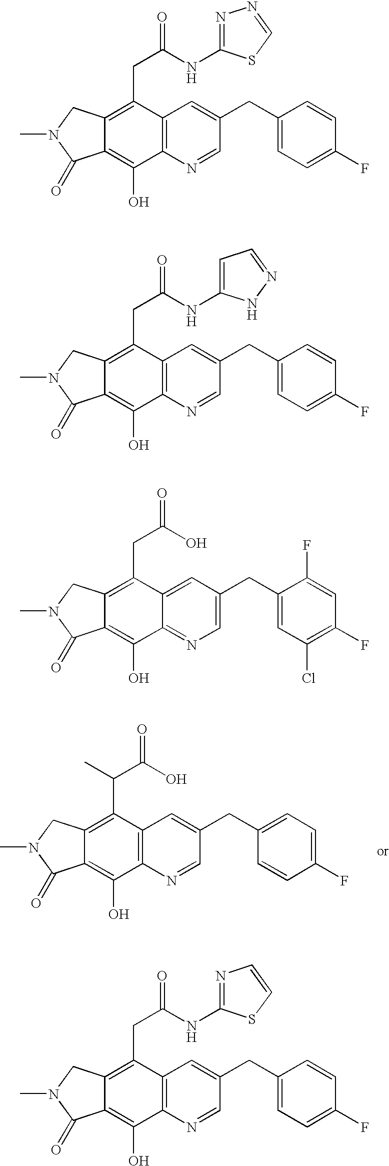
or a pharmaceutically acceptable salt or prodrug thereof.

31. The compound 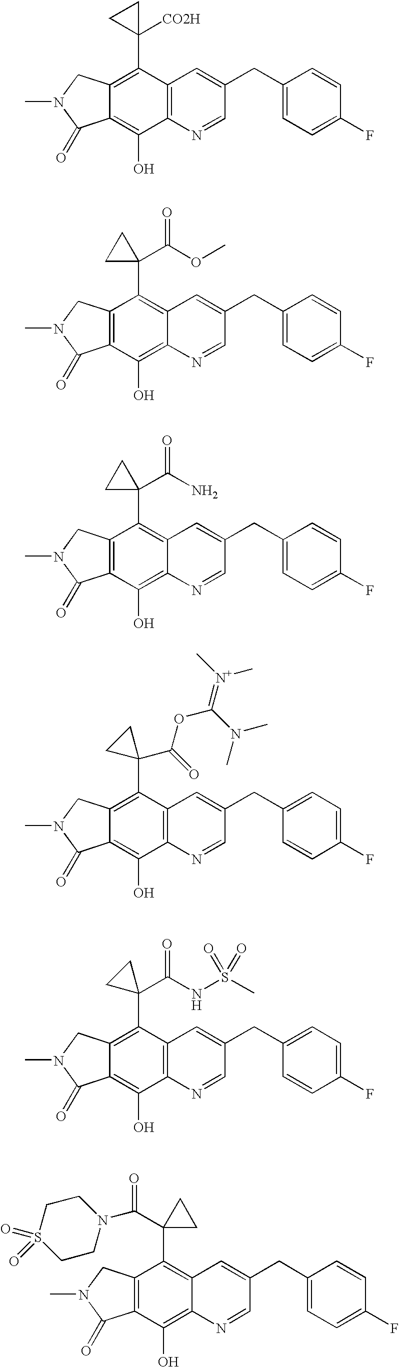 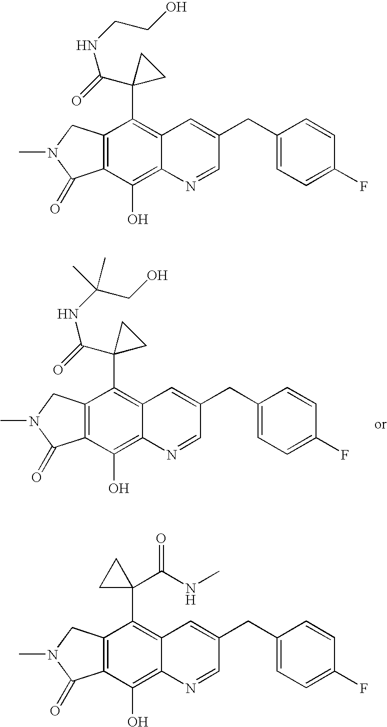
or a pharmaceutically acceptable salt or prodrug thereof.

32. The compound 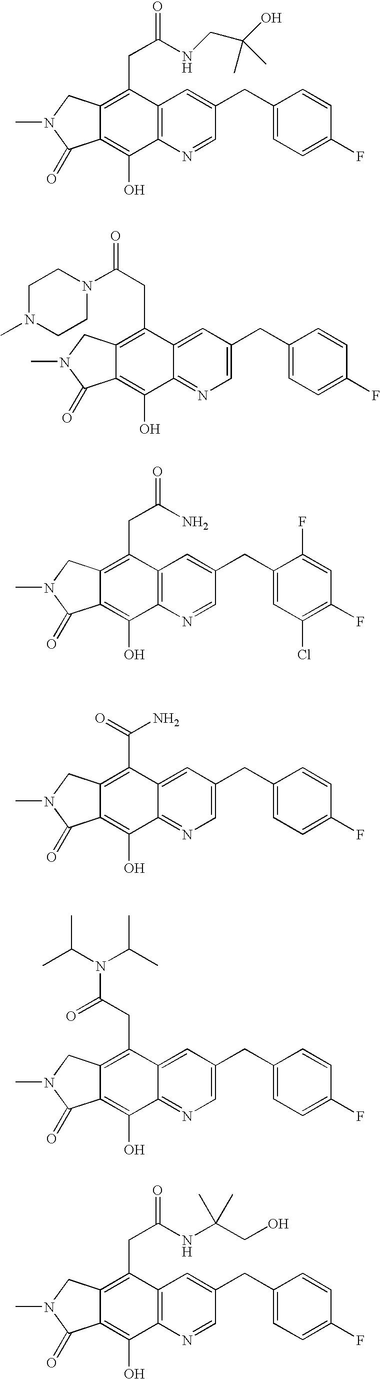 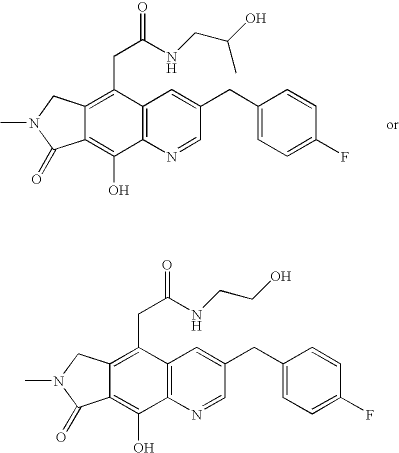
or a pharmaceutically acceptable salt or prodrug thereof.

33. A prodrug of the compound of claim 1.

34. A phosphonate which is a compound of claim 1 wherein at least one hydrogen atom is replaced with a group A⁵, wherein each A⁵ is independently: 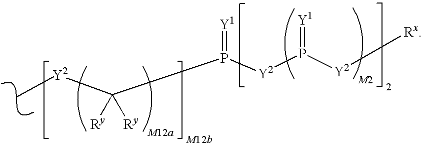
Y¹ is independently O, S, N(R^{x}), N(O)(R^{x}), N(OR^{x}), N(O)(OR^{x}), or N(N(R^{x})₂.
Y² is independently a bond, O, N(R^{x}), N(O)(R^{x}), N(OR^{x}), N(O)(OR^{x}), N(N(R^{x})₂), —S(O)— (sulfoxide), —S(═O)₂— (sulfone), —S-(sulfide), or —S—S-(disulfide).
M2 is 0, 1 or 2.
M12a is 1, 2, 3, 4, 5, 6, 7, 8, 9, 10, 11, or 12.
M12b is 0, 1, 2, 3, 4, 5, 6, 7, 8, 9, 10, 11, or 12.
R^{y} is independently H, C₁-C₆ alkyl, C₁-C₆ substituted alkyl, aryl, substituted aryl, or a protecting group. Alternatively, taken together at a carbon atom, two vicinal R^{y} groups form a ring, i.e. a spiro carbon. The ring may be all carbon atoms, for example, cyclopropyl, cyclobutyl, cyclopentyl, or cyclohexyl, or alternatively, the ring may contain one or more heteroatoms, for example, piperazinyl, piperidinyl, pyranyl, or tetrahydrofuryl.
R^{x} is independently H, C₁-C₆ alkyl, C₁-C₆ substituted alkyl, C₆-C₂₀ aryl, C₆-C₂₀ substituted aryl, or a protecting group, or the formula: 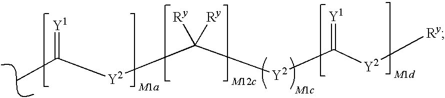
M1a, M1c, and M1d are independently 0 or 1; and
M12c is 0, 1, 2, 3, 4, 5, 6, 7, 8, 9, 10, 11 or 12.

35. The phosphonate of claim 34 which is a prodrug.

36. A pharmaceutical composition comprising the compound or pharmaceutically acceptable salt according to claim 1 and a pharmaceutically acceptable excipient, diluent or carrier.

37. The pharmaceutical composition of claim 36, further comprising an AIDS treatment agent, an anti-infective agent, an immunomodulator agent a booster agent or a mixture thereof.

38. The pharmaceutical composition of claim 37, where the AIDS treatment agent is an HIV-protease inhibitor, a nucleoside reverse transcriptase inhibitor, a non-nucleoside reverse transcriptase inhibitor or a mixture thereof.

39. The pharmaceutical composition of claim 36 which is in an oral dosage form.

40. A method of treating the proliferation of HIV virus, treating AIDS, or delaying the onset of AIDS or ARC symptoms, comprising administering to a mammal in need thereof, a therapeutically effective amount of the compound of claim 1.

41. A method of inhibiting HIV integrase, comprising administering to a mammal in need thereof, a therapeutically effective amount of the compound of claim 1.

42. The method of claim 41, further comprising administering to a mammal in need thereof, a booster agent, a therapeutically effective amount of an AIDS treatment agent, a therapeutically effective amount of an anti-infective agent, a therapeutically effective amount of an immunomodulator agent, or a mixture thereof.

43. A kit for the treatment of disorders, symptoms and diseases where integrase inhibition plays a role, comprising two or more separate containers in a single package, wherein at least one compound or pharmaceutically acceptable salt of claim 1 is placed in combination with one or more of the following: a pharmaceutically acceptable carrier, a booster agent, a therapeutically effective amount of an AIDS treatment agent, a therapeutically effective amount of an anti-infective agent or a therapeutically effective amount of an immunomodulator agent.

44. The compound or pharmaceutically acceptable salt of claim 1 for use in therapy.

45. Use of the compound or pharmaceutically acceptable salt of claim 1 in the manufacture of a medicament for the treatment of HIV.

46. A compound, pharmaceutically acceptable salt or pharmaceutical composition as described herein.

47. A method of promoting an antiviral effect in an animal comprising administering to the animal an effective amount of the compound of claim 1.
